(19) United States
(12) Patent Application Publication
(10) Pub. No.: US 2026/0318927 A1
(43) Pub. Date: Oct. 15, 2026

(54) MID-REGIONAL PRO-ADRENOMEDULLIN IMMUNOASSAY WITH MONOCLONAL ANTIBODIES

(71) Applicants: B.R.A.H.M.S GMBH, Hennigsdorf (DE); CEZANNE S.A.S., Nimes Cédex 1 (FR)

(72) Inventors: Karin LEHMANN, Hennigsdorf (DE); Manne KROP, Hennigsdorf (DE); Carola WUNDERLICH, Hennigsdorf (DE); Anne INCAMPS, Saint-Victor-la-Coste (FR); Lucie ROUX, Nîmes (FR); Nicolas BOURGOIN, Bernis (FR)

(73) Assignees: B.R.A.H.M.S GMBH, Hennigsdorf (DE); CEZANNE S.A.S., Nimes Cédex 1 (FR)

(21) Appl. No.: 19/483,720

(22) PCT Filed: May 10, 2024
(86) PCT No.: PCT/EP2024/062914

§ 371 Date: Nov. 12, 2025

(30) Foreign Application Priority Data
May 12, 2023 (EP) 23305751.2
Oct. 25, 2023 (EP) 23205939.4

(51) Int. Cl. G01N 33/74; C07K 16/22; G01N 33/58

(52) U.S. Cl. CPC G01N 33/74; C07K 16/22; G01N 33/582; C07K 2317/524; C07K 2317/526; C07K 2317/55; C07K 2317/565; C07K 2317/92; C07K 2317/94

(57) ABSTRACT

The invention relates to a method for determining mid-regional pro-adrenomedullin (MR-proADM) in a sample, comprising two labelled monoclonal antibodies that bind (preferably distinct) epitopes of MR-proADM, and detecting a signal by the binding of both antibodies to MR-proADM. The method relates further to a monoclonal antibody or antibody fragment that binds mid-regional pro-adrenomedullin (MR-proADM). The invention further relates to diagnostic uses and methods, such as an in vitro method for determining MR-proADM in a sample using an antibody or antibody fragment of the invention.

The invention relates to the field of diagnostic reagents, immunoassays and antibodies for measuring or determining biomarkers in samples.

The invention relates to a method for determining mid-regional pro-adrenomedullin (MR-proADM) in a sample, comprising two labelled monoclonal antibodies that bind (preferably distinct) epitopes of MR-proADM, and detecting a signal by the binding of both antibodies to MR-proADM.

One aspect relates to an in vitro method for determining mid-regional pro-adrenomedullin (MR-proADM) in a sample, comprising contacting the sample with (i.) a first monoclonal antibody or fragment thereof, specific for a first epitope of MR-proADM, and (ii.) a second monoclonal antibody or fragment thereof, specific for a second epitope of MR-proADM; and detecting the binding of the first and second monoclonal antibodies or fragments thereof to said MR-proADM, wherein the first and second antibodies are labeled with detectable labels, wherein a signal generated by said labels is modulated when the first and second antibodies are bound in an antibody-MR-proADM complex.

In preferred embodiments, proximity of donor and acceptor labels in an antibody-MR-proADM complex and a spectral overlap between a donor emission and acceptor absorption spectra of said labels intensifies the signal and/or extends the life span of the signal allowing for the measurement of temporally delayed fluorescence. In other preferred embodiments, the donor label comprises a terbium cryptate or chelate and the acceptor label comprises a fluorescent dye with excitation and emission spectra compatible with the terbium excitation and emission spectra (in a time-resolved fluorescence resonance energy transfer (TR-FRET) reaction and/or a time-resolved amplified cryptate emission (TRACE) reaction).

The invention further relates to a monoclonal antibody or antibody fragment that binds mid-regional pro-adrenomedullin (MR-proADM).

The invention further relates to diagnostic uses and methods, such as an in vitro method for determining MR-proADM in a sample using an antibody or antibody fragment of the invention, and to medical uses and corresponding therapeutic methods of administering an antibody or antibody fragment of the invention in the treatment and/or prevention of a medical condition.

In further aspects, the invention relates to a nucleic acid molecule comprising a nucleotide sequence that encodes an antibody or antibody fragment of the invention, a host cell comprising said nucleic acid molecule, a host cell capable of producing an antibody or antibody fragment of the invention and a composition, such as a pharmaceutical composition, comprising an antibody or antibody fragment of the invention.

### BACKGROUND OF THE INVENTION

Adrenomedullin (ADM) is a 52-amino acid peptide described as having a variety of physiologic functions. Its strong vasodilatory activity has been described in several studies. Measurement of ADM has been shown to be useful in various clinical diagnostic and prognostic settings, for example in the diagnosis, monitoring, and prognosis of various cardiovascular diseases and sepsis, for which increased plasma concentrations of ADM have been described.

However, the reliable measurement of ADM in blood circulation is difficult. In addition to immediate binding of ADM to receptors in the vicinity of its production, via autocrine and/or paracrine reactions, peripheral measurement is also hampered by the existence of a binding protein, the short half-life of ADM, and technical difficulties. ADM is derived from a larger precursor peptide (preproADM; 185 amino acids) by posttranslational processing. Cleavage of pre-pro-ADM leads to various smaller peptides being generating, such as PAMP, mature (bio) ADM or mid-regional proADM (MR-proADM).

Methods for detecting proADM, and in particular MR-proADM, have been described previously, although these approaches rely on polyclonal antibodies derived for example from immunization in sheep. Morgenthaler (Clinical Chemistry 51:10; 1823-1829, 2005) describes the technical characterization of a sandwich immunoassay for the measurement of MR-proADM in human plasma, its reference interval in healthy individuals, and the finding of increased plasma concentrations in patients with cardiovascular disease or sepsis.

Despite the effectiveness of such assays, polyclonal antibodies are potentially problematic due to cross-reactivity and limited stocks. The B⋅R⋅A⋅H⋅M⋅S MR-proADM KRYPTOR™ system enables measurement of MR-proADM concentrations and provides a more precise assessment of disease severity and patient risk management and can enhance clinical investigation and treatment decisions. In the intensive care department, it can provide an immediate assessment of disease severity to maximise patient safety, guide the most appropriate treatment and provide an early warning of developing complications.

The B⋅R⋅A⋅H⋅M⋅S KRYPTOR Analysers are fully automatic, closed laboratory analysis systems that can perform numerous analyses in random-access operations using a unique measuring principle called TRACE™ (Time Resolved Amplified Cryptate Emission). Nevertheless, the KRYPTOR technology relies on an immunoassay concept and requires sensitive, specific, and highly affine antibodies in order to enable accurate MR-proADM determination in clinical diagnostics.

Caruhel et al (Clinical Biochemistry 42 (2009) 725-728) describe the B⋅R⋅A⋅H⋅M⋅S KRYPTOR system and the analysis of EDTA plasma samples from patients with community acquired pneumonia. The system uses the TRACE technology and is based on a nonradiative energy transfer between a donor, the long-lived fluorophore Europium Cryptate (EuC) and an acceptor, Cyanine 5 (Cy5). The method described in Caruhel et al relies however on polyclonal MR-proADM antibodies and thus suffers from the common disadvantages of such systems.

US 2019/178897 discloses a method for diagnosing organ dysfunction, comprising the measurement of proADM and histones in samples from patients. The use of a KRYPTOR device is disclosed therein.

Despite immunoassays for MR-proADM being available on the market, the need exists for alternative or improved immunoassays and antibodies that overcome the disadvantages of the prior art, such as those associated with polyclonal antibodies (cross-reactivity and limited stocks), and to provide immunoassays and antibodies that are more sensitive and specific than those described previously, thus enabling improved detection of the MR-proADM biomarker.

### SUMMARY OF THE INVENTION

In light of the prior art, the technical problem underlying the invention was the provision of alternative or improved means for determining and/or measuring MR-proADM and/or diagnosing and/or treating medical conditions associated with MR-proADM.

A further object of the invention was the provision of an immunoassay that overcomes the deficiencies of the prior art. One object of the invention was to provide a more sensitive immunoassay than those previously available. One object of the invention was to provide a sensitive immunoassay, preferably a more sensitive immunoassay or at least an assay with the same sensitivity as those previously available, with improved or equivalent accuracy in quantitation of MR-proADM in a patient sample. One object of the invention was to provide a higher level of signal RFU than those previously available, with improved or equivalent accuracy in quantitation of MR-proADM in a patient sample.

A further object of the invention was the provision of an antibody or antibody fragment, with improved properties.

These problems are solved by the features of the independent claims. Preferred embodiments of the present invention are provided by the dependent claims.

One aspect of the invention therefore relates to an in vitro method for determining mid-regional pro-adrenomedullin (MR-proADM) in a sample, comprising:
contacting the sample with
   i. a first monoclonal antibody or fragment thereof, specific for a first epitope of MR-proADM, and
   ii. a second monoclonal antibody or fragment thereof, specific for a second epitope of MR-proADM; and
detecting the binding of the first and second monoclonal antibodies or fragments thereof to said MR-proADM,
wherein the first and second antibodies are labeled with detectable labels, wherein a signal generated by said labels is modulated when the first and second antibodies are bound in an antibody-MR-proADM complex.

As described in more detail below, the method and immunoassay of the present invention represent an improved version of the existing B⋅R⋅A⋅H⋅M⋅S MR-proADM system, in which the polyclonal antibodies employed in the previous system are replaced using monoclonal antibodies with improved properties, leading to a system with improved sensitivity and signal strength compared to the earlier system.

As described in Morgenthaler (Clinical Chemistry 51:10; 1823-1829, 2005) polyclonal antibodies were generated and effectively employed to enable the present B⋅R⋅A⋅H⋅M⋅S MR-proADM system. Three peptides related to preproADM were chemically synthesized, purified, and sheep were immunized with the peptides and antibodies subsequently purified using the corresponding peptides immobilized on a solid phase.

The present invention has employed distinct antigens and epitopes to produce murine antibodies, which have then been isolated and developed as monoclonal antibodies and tested in similar assay formats as the established B⋅R⋅A⋅H⋅M⋅S MR-proADM system. Surprisingly, the use of two monoclonal antibodies as in the present invention leads to significantly improved results over the earlier polyclonal technology. The accuracy in MR-proADM quantification in patient samples is maintained over the older technology, although unexpectedly a significantly improved RFU signal is obtained using the monoclonal antibody approach.

The epitopes targeted by the inventive monoclonal antibodies are described in more detail below.

In one embodiment, the first antibody and second antibody are dispersed in a liquid reaction mixture (homogenous immunoassay).

In one embodiment, the first antibody is labelled with a donor label and the second antibody is labelled with an acceptor label.

In one embodiment, proximity of donor and acceptor labels in the antibody-MR-proADM complex and a spectral overlap between a donor emission and acceptor absorption spectra intensifies the signal and/or extends the life span of the signal allowing for the measurement of temporally delayed fluorescence.

In embodiments, the present invention employs a time-resolved fluorescence resonance energy transfer (TR-FRET) reaction or a time-resolved amplified cryptate emission (TRACE) reaction.

In embodiments, the donor label comprises a rare earth cryptate or chelate, preferably comprising a lanthanide ion, and the acceptor label comprises a fluorescent or chemiluminescent dye.

In preferred embodiments, the first antibody is labelled with a donor label and the second antibody is labelled with an acceptor label, wherein proximity of donor and acceptor labels in an antibody-MR-proADM complex, and a spectral overlap between a donor emission and acceptor absorption spectra, modulates the signal (e.g., intensifies the signal and/or extends the life span of the signal allowing for the measurement of temporally delayed fluorescence), wherein the donor label comprises a rare earth cryptate or chelate, and the acceptor label comprises a fluorescent or chemiluminescent dye.

In preferred embodiments, the method of the present invention thus employs the TRACE technology of the KRYPTOR system, using a lanthanide ion, and the acceptor label comprises a fluorescent or chemiluminescent dye, although coupled to monoclonal antibodies targeting unique combinations of MR-proADM epitopes.

Lanthanide luminescence is established in the art and offers several advantages for fluorescence-based biological assays, including large Stoke's shifts (>150 nm) and multiple, narrow emission bands (typically <10 nm at half-maximum) and thus allows efficient spectral separation of emission signals. Lanthanide luminescence is also typically characterized by long luminescence lifetimes (micro- to millisecond) enabling time-resolved detection methods. Lanthanide ions (particularly Terbium and Europium) offer significant advantages over organic fluorescent dyes. Compared with traditional organic fluorescent materials, the lanthanide coordination compounds have a relatively long life-time, a large Stokes shift, and sharp fluorescent peak profiles. Due to these attractive properties, time-resolved reagents based on lanthanide fluorescence have been created.

Terbium and europium probes typically incorporate the metal ion into an organic chelating ligand that contains a sensitizing chromophore. When excited with near-UV light in the absorption band, the chromophore transfers energy via intersystem crossing to the triplet excited state and intramolecular transfer to the emissive level of the chelated metal. Direct conjugation of lanthanide probes to antibodies thus enables the development of sensitive, time-resolved fluorescence resonance energy transfer (TR-FRET) or time-resolved amplified cryptate emission (TRACE) assays in various practical applications. The B⋅R⋅A⋅H⋅M⋅S MR-proADM KRYPTOR system employs such TRACE technology, which is also employed in preferred embodiments of the present invention.

Surprisingly, the signal intensity obtained in the inventive method and system, based on labeled monoclonal antibodies, cold be enhanced over older similar assays based on polyclonal antibodies. Furthermore, the use of terbium labels achieved an additional increase in signal strength in appropriate assay formats.

In one embodiment, the donor label comprises a terbium cryptate or chelate.

In embodiments, a donor label may comprise terbium, terbium-derivatives, or a terbium N-hydroxy succinimide ester, for example C₆₅H₇₈N₁₄O₁₇Tb.

In one embodiment, the donor label comprises a terbium cryptate or chelate and the acceptor label comprises a fluorescent dye with excitation and emission spectra compatible with the terbium excitation and emission spectra (in a time-resolved fluorescence resonance energy transfer (TR-FRET) reaction and/or a time-resolved amplified cryptate emission (TRACE) reaction).

As used herein, the term “compatible with the terbium excitation and emission spectra”, or “compatible with the terbium excitation and emission spectra in a time-resolved fluorescence resonance energy transfer (TR-FRET) reaction and/or a time-resolved amplified cryptate emission (TRACE) reaction”, refers to the election of suitable fluorophores with complementary (compatible) excitation and emission spectra that are receptive to effective energy transfer and subsequent detection in a time-resolved method (FRET or TRACE). By way of example, a terbium donor label is employed, and the acceptor label is selected to have a matching excitation spectrum to be excited by the donor upon binding, and an emission spectrum that can be detected at a suitable wavelength.

By way of example, the present method is based on and represents a further development of an older method using radiative energy transfer between a donor, the long-lived fluorophore Europium Cryptate, and an acceptor, such as a fluorophore emitting in the red wavelength, such as the Cyanine 5 (Cy5). The energy transfer depends on the proximity and the overlap of spectrum of the two fluorophores. In a sandwich immunoassay, the Europium and acceptor are bound to specific antibodies. In the older assay, the Europium is excited at 337 nm by a nitrogen laser and the energy is transferred to the Cy5 which reemits specifically at 665 nm. This homogeneous assay uses time-resolved measurements and spectral selection to eliminate the background signal of the media and to select the specific signal.

The present invention therefore enables a surprising development over this technology, by employing preferably terbium in place of europium and/or by employing novel monoclonal antibodies, thus enabling unexpected improvements over the earlier technology.

In one embodiment, the donor label comprises a terbium cryptate or chelate with emission spectra at 485-495, 540-550, 585-595 and/or 615-625 nm, and the acceptor label comprises a fluorescent dye with a excitation spectrum corresponding to the donor label emission spectrum, and an emission spectrum distinct from the donor label emission spectra, preferably in a wavelength range of 500-570, preferably 510-560, or 515-555 nm (green), or 580-770, preferably 600-780, or 670-690 nm (red).

Suitable acceptor fluorophores may be elected based on compatible excitation and emission spectra, and are disclosed in more detail below.

In preferred embodiments, the method comprises a fully automated homogeneous assay, preferably a homogeneous sandwich fluoroimmunoassay, employing a Time Resolved Amplified Cryptate Emission (TRACE) technology with a non-radiative energy transfer between a donor and an acceptor, wherein the energy transfer depends on the proximity and the overlap of spectrum of the two fluorophores.

In embodiments, the first and/or second antibody or fragment thereof is a murine antibody, preferably a mouse antibody, for example wherein the antibody of fragment thereof comprises or consists of an amino acid sequence isolated after immunization of a murine subject.

In embodiments, the first and second epitopes are different from each other and selected from MR-proADM epitopes according to SEQ ID NO 5-18.

As described below, various monoclonal antibodies have been produced and validated in the present system. The antibodies may be defined by their sequence, the relevant epitopes of MR-proADM to which they bind, or any one or more of the structural and/or functional properties as described herein. The antibodies are named as Ab1-7 and described in detail below.

In one embodiment, the antibody “Ab1” binds an epitope according to SEQ ID NO 5 or 6.

In one embodiment, the antibody “Ab2” binds an epitope according to SEQ ID NO 7 or 8.

In one embodiment, the antibody “Ab3” binds an epitope according to SEQ ID NO 9 or 10.

In one embodiment, the antibody “Ab4” binds an epitope according to SEQ ID NO 11 or 12.

In one embodiment, the antibody “Ab5” binds an epitope according to SEQ ID NO 13 or 14.

In one embodiment, the antibody “Ab6” binds an epitope according to SEQ ID NO 15 or 16.

In one embodiment, the antibody “Ab7” binds an epitope according to SEQ ID NO 17 or 18.

In embodiments, preferred combinations of antibodies, to be employed in the methods of the present invention, are:
Ab1 with Ab2 or Ab3, preferably Ab1 with Ab2,
Ab2 with Ab1, Ab4, Ab3, Ab5 or Ab6, preferably Ab2 with Ab3,
Ab4 with Ab2,
Ab3 with Ab1, Ab2, or Ab7,
Ab5 with Ab2,
Ab6 with Ab2, and
Ab7 with Ab3.

### Embodiments with Specific Epitope and/or Antibody Sequences

In embodiments, the first and/or second antibody comprises VH and VL domains that comprise the sequences according to SEQ ID NO 136 and 137 (Ab1), 151 and 152 (Ab2), 166 and 167 (Ab3), 181 and 182 (Ab4), 196 and 197 (Ab5), 211 and 212 (Ab6), or 226 and 227 (Ab7), respectively.

In embodiments, the first and second antibodies may be any combination of the antibodies described above (or elsewhere herein).

The following combinations of particular embodiments are preferred, without intending limitation to any one or more of these specific combinations.

### Ab2 and Ab3:

In one embodiment, the first and second epitopes are different and selected from SEQ ID NO 7 or 8 (epitope of Ab2), and SEQ ID NO 9 or 10 (epitope of Ab3), and wherein the donor label comprises a terbium cryptate or chelate, and the acceptor label comprises a fluorescent dye with excitation and emission spectra compatible with the terbium excitation and emission spectra (in a time-resolved fluorescence resonance energy transfer (TR-FRET) reaction and/or a time resolved amplified cryptate emission (TRACE) reaction, preferably according to claim 7).

In one embodiment, the first and second antibodies are different, and each comprise 6 CDR sequences, according to:
H-CDR1 according to SEQ ID NO 153 or 154, H-CDR2 according to SEQ ID NO 155 or 156, H-CDR3 according to SEQ ID NO 157 or 158, L-CDR1 according to SEQ ID NO 159 or 160, L-CDR2 according to SEQ ID NO 161 or 162, and L-CDR3 according to SEQ ID NO 163 (Ab2), and
H-CDR1 according to SEQ ID NO 168 or 169, H-CDR2 according to SEQ ID NO 170 or 171, H-CDR3 according to SEQ ID NO 172 or 173, L-CDR1 according to SEQ ID NO 174 or 175, L-CDR2 according to SEQ ID NO 176 or 177, and L-CDR3 according to SEQ ID NO 178 (Ab3),
and wherein the donor label comprises a terbium cryptate or chelate, and the acceptor label comprises a fluorescent dye with excitation and emission spectra compatible with the terbium excitation and emission spectra (in a time-resolved fluorescence resonance energy transfer (TR-FRET) reaction and/or a time resolved amplified cryptate emission (TRACE) reaction, preferably according to claim 7).

In one embodiment, the first and second antibodies are different and comprise VH and VL domains according to SEQ ID NO 151 and 152 (Ab2), and SEQ ID NO 166 and 167 (Ab3).

### Ab2 and Ab4:

In one embodiment, the first and second epitopes are different and selected from SEQ ID NO 7 or 8 (epitope of Ab2), and SEQ ID NO 11 or 12 (epitope of Ab4), and wherein the donor label comprises a terbium cryptate or chelate and the acceptor label comprises a fluorescent dye with excitation and emission spectra compatible with the terbium excitation and emission spectra (in a time-resolved fluorescence resonance energy transfer (TR-FRET) reaction and/or a time resolved amplified cryptate emission (TRACE) reaction, preferably according to claim 7).

In one embodiment, the first and second antibodies are different, and each comprise 6 CDR sequences, according to:
H-CDR1 according to SEQ ID NO 153 or 154, H-CDR2 according to SEQ ID NO 155 or 156, H-CDR3 according to SEQ ID NO 157 or 158, L-CDR1 according to SEQ ID NO 159 or 160, L-CDR2 according to SEQ ID NO 161 or 162, and L-CDR3 according to SEQ ID NO 163 (Ab2), and
H-CDR1 according to SEQ ID NO 183 or 184, H-CDR2 according to SEQ ID NO 185 or 186, H-CDR3 according to SEQ ID NO 187 or 188, L-CDR1 according to SEQ ID NO 189 or 190, L-CDR2 according to SEQ ID NO 191 or 192, and L-CDR3 according to SEQ ID NO 193 (Ab4),
and wherein the donor label comprises a terbium cryptate or chelate, and the acceptor label comprises a fluorescent dye with excitation and emission spectra compatible with the terbium excitation and emission spectra (in a time-resolved fluorescence resonance energy transfer (TR-FRET) reaction and/or a time resolved amplified cryptate emission (TRACE) reaction, preferably according to claim 7).

In one embodiment, the first and second antibodies are different and comprise VH and VL domains according to SEQ ID NO 151 and 152 (Ab2), and SEQ ID NO 181 and 182 (Ab4).

### Ab1 and Ab2:

In one embodiment, the first and second epitopes are different and selected from SEQ ID NO 5 or 6 (epitope of Ab1), and SEQ ID NO 7 or 8 (epitope of Ab2), and wherein the donor label comprises a terbium cryptate or chelate and the acceptor label comprises a fluorescent dye with excitation and emission spectra compatible with the terbium excitation and emission spectra (in a time-resolved fluorescence resonance energy transfer (TR-FRET) reaction and/or a time resolved amplified cryptate emission (TRACE) reaction, preferably according to claim 7).

In one embodiment, the first and second antibodies are different, and each comprise 6 CDR sequences, according to:
H-CDR1 according to SEQ ID NO 138 or 139, H-CDR2 according to SEQ ID NO 140 or 141, H-CDR3 according to SEQ ID NO 142 or 143, L-CDR1 according to SEQ ID NO 144 or 145, L-CDR2 according to SEQ ID NO 146 or 147, and L-CDR3 according to SEQ ID NO 148 (Ab1), and
H-CDR1 according to SEQ ID NO 153 or 154, H-CDR2 according to SEQ ID NO 155 or 156, H-CDR3 according to SEQ ID NO 157 or 158, L-CDR1 according to SEQ ID NO 159 or 160, L-CDR2 according to SEQ ID NO 161 or 162, and L-CDR3 according to SEQ ID NO 163 (Ab2),
and wherein the donor label comprises a terbium cryptate or chelate, and the acceptor label comprises a fluorescent dye with excitation and emission spectra compatible with the terbium excitation and emission spectra (in a time-resolved fluorescence resonance energy transfer (TR-FRET) reaction and/or a time resolved amplified cryptate emission (TRACE) reaction, preferably according to claim 7).

In one embodiment, the first and second antibodies are different and comprise VH and VL domains according to SEQ ID NO 136 and 137 (Ab1), 151 and 152 (Ab2).

### Ab1 and Ab3:

In one embodiment, the first and second epitopes are different and selected from SEQ ID NO 5 or 6 (epitope of Ab1), and SEQ ID NO 9 or 10 (epitope of Ab3), and wherein the donor label comprises a terbium cryptate or chelate and the acceptor label comprises a fluorescent dye with excitation and emission spectra compatible with the terbium excitation and emission spectra (in a time-resolved fluorescence resonance energy transfer (TR-FRET) reaction and/or a time resolved amplified cryptate emission (TRACE) reaction, preferably according to claim 7).

In one embodiment, the first and second antibodies are different, and each comprise 6 CDR sequences, according to:
H-CDR1 according to SEQ ID NO 138 or 139, H-CDR2 according to SEQ ID NO 140 or 141, H-CDR3 according to SEQ ID NO 142 or 143, L-CDR1 according to SEQ ID NO 144 or 145, L-CDR2 according to SEQ ID NO 146 or 147, and L-CDR3 according to SEQ ID NO 148 (Ab1), and
H-CDR1 according to SEQ ID NO 168 or 169, H-CDR2 according to SEQ ID NO 170 or 171, H-CDR3 according to SEQ ID NO 172 or 173, L-CDR1 according to SEQ ID NO 174 or 175, L-CDR2 according to SEQ ID NO 176 or 177, and L-CDR3 according to SEQ ID NO 178 (Ab3),
and wherein the donor label comprises a terbium cryptate or chelate, and the acceptor label comprises a fluorescent dye with excitation and emission spectra compatible with the terbium excitation and emission spectra (in a time-resolved fluorescence resonance energy transfer (TR-FRET) reaction and/or a time resolved amplified cryptate emission (TRACE) reaction, preferably according to claim 7).

In one embodiment, the first and second antibodies are different and comprise VH and VL domains according to SEQ ID NO 136 and 137 (Ab1), and SEQ ID NO 166 and 167 (Ab3).

In embodiments, the antibodies employed in the embodiments described above (or elsewhere herein) may be defined by the epitopes that are bound by said antibodies. For example, the epitopes bound by Ab1-4 represent unique, novel epitopes over the prior art, which enable unexpectedly effective sensitivity in MR-proADM detection in TRACE-based detection systems. There is no suggestion in the art of employing the antigens and epitopes as disclosed for the inventive antibodies, and it was surprising that enhanced TRACE signals were obtained using the labels and detection systems described herein.

In embodiments, the antibodies employed in the embodiments described above (or elsewhere herein) may be defined by the CDR sequences of the respective antibodies, as disclosed herein, either in combination with or independently of the complete specific VH and VL sequences. For example, the CDR sequences of each of Ab1, Ab2, Ab3, Ab4 are disclosed in the sequence table of the present application.

In embodiments, the antibodies may be defined by a combination of CDR sequences of the respective antibodies, together with sequences with at least 70%, 80%, 90% or 95% sequence identity to the specific VH and VL sequences of the respective antibodies.

### Embodiments Using Alternative Antibody Sequences

In embodiments, the first and/or second antibody comprises VH and VL domains that comprise the sequences according to SEQ ID NO 21 and 22 (Ab1), 39 and 40 (Ab2), 54 and 55 (Ab3), 68 and 69 (Ab4), 84 and 85 (Ab5), 102 and 103 (Ab6), or 119 and 120 (Ab7), respectively.

In one embodiment, the first and second antibodies are different and comprise VH and VL domains according to SEQ ID NO 39 and 40 (Ab2), and SEQ ID NO 54 and 55 (Ab3), and wherein the donor label comprises a terbium cryptate or chelate, and the acceptor label comprises a fluorescent dye with excitation and emission spectra compatible with the terbium excitation and emission spectra (in a time-resolved fluorescence resonance energy transfer (TR-FRET) reaction and/or a time resolved amplified cryptate emission (TRACE) reaction).

In one embodiment, the first and second antibodies are different and comprise VH and VL domains according to SEQ ID NO 39 and 40 (Ab2), and SEQ ID NO 68 and 69 (Ab4), and wherein the donor label comprises a terbium cryptate or chelate and the acceptor label comprises a fluorescent dye with excitation and emission spectra compatible with the terbium excitation and emission spectra (in a time-resolved fluorescence resonance energy transfer (TR-FRET) reaction and/or a time resolved amplified cryptate emission (TRACE) reaction).

In one embodiment, the first and second antibodies are different and comprise VH and VL domains according to SEQ ID NO 21 and 22 (Ab1), and SEQ ID NO 54 and 55 (Ab3), and wherein the donor label comprises a terbium cryptate or chelate, and the acceptor label comprises a fluorescent dye with excitation and emission spectra compatible with the terbium excitation and emission spectra (in a time-resolved fluorescence resonance energy transfer (TR-FRET) reaction and/or a time resolved amplified cryptate emission (TRACE) reaction).

In one embodiment, the first and second antibodies are different and comprise VH and VL domains according to SEQ ID NO 21 and 22 (Ab1), and SEQ ID NO 39 and 40 (Ab2), and wherein the donor label comprises a terbium cryptate or chelate, and the acceptor label comprises a fluorescent dye with excitation and emission spectra compatible with the terbium excitation and emission spectra (in a time-resolved fluorescence resonance energy transfer (TR-FRET) reaction and/or a time resolved amplified cryptate emission (TRACE) reaction).

In embodiments, the antibodies employed in the embodiments described above (or elsewhere herein) may be defined by the CDR sequences of the respective antibodies, as disclosed herein, either in combination with or independently of the complete specific VH and VL sequences.

In embodiments, the antibodies may be defined by a combination of CDR sequences of the respective antibodies, together with sequences with at least 70%, 80%, 90% or 95% sequence identity to the specific VH and VL sequences of the respective antibodies.

### Further Embodiments

Corresponding embodiments for any one or more of Ab1-7, in any given combination, preferably those disclosed herein, are covered by the invention.

As shown in the examples below, various embodiments, for different combinations of antibodies and fluorophores, especially the preferred combinations disclosed herein, achieve relatively greater signal measured in ratio fluorescent units (RFU) over the prior art B⋅R⋅A⋅H⋅M⋅S MR-proADM assay. Due to these properties, a greater signal emittance may enable improvements in a lower detection limit, an intraassay precision and/or a functional assay sensitivity over prior art assays. Additionally, no loss in accuracy is obtained, and the absolute MR-proADM concentration determination shows high concurrence with the existing assay, thus the present invention may achieve similarly reliable accuracy in determining MR-proADM but exhibit advantages due to the more sensitive detection.

In embodiments, the antibodies and fluorophores employed enable improvements in the lower detection limit compared to earlier assays based on alternative antibodies (e.g., polyclonal antibodies) and/or alternative fluorophores.

By way of example, in embodiments, a lower detection limit of the present assay, using methods as described in the reference paper Morgenthaler (Clinical Chemistry 51:10; 1823-1829, 2005), for example as determined with horse serum (mean relative light units of 20 determinations plus 2 SD), is less than 0.08 nmol/L, preferably less than 0.05 nmol/L, more preferably less than 0.01 nmol/L, or 0.005 nmol/L.

In embodiments, the antibodies and fluorophores employed enable improvements in the intraassay precision compared to earlier assays based on alternative antibodies (e.g., polyclonal antibodies) and/or alternative fluorophores.

By way of example, in embodiments, a intraassay precision of the present assay, using methods as described in the reference paper Morgenthaler (Clinical Chemistry 51:10; 1823-1829, 2005), for example as determined by measuring multiple (e.g., 10-30) human EDTA plasma samples covering a concentration range of e.g., 0.01-20.00 nmol/L in multiple (e.g., 5-15) parallel measurements, is less than 5%, preferably less than 4%, 3%, 2%, or less than 1%.

In embodiments, the antibodies and fluorophores employed enable improvements in the functional assay sensitivity compared to earlier assays based on alternative antibodies (e.g., polyclonal antibodies) and/or alternative fluorophores.

By way of example, in embodiments, a functional assay sensitivity of the present assay, using methods as described in the reference paper Morgenthaler (Clinical Chemistry 51:10; 1823-1829, 2005), shows an improvement over the prior art. For example, the functional assay sensitivity may be considered as the MR-proADM concentration with an inter-assay precision of 20%.

In one aspect, the invention further relates to an analytical, detection and/or diagnostic kit for use in the method of the invention, comprising:
a first monoclonal antibody and a second monoclonal antibody, or fragments thereof, that bind a first and second epitope of MR-proADM, respectively, and
one or more donor labels comprising a rare earth cryptate or chelate, preferably comprising a lanthanide ion, more preferably comprising a Terbium chelate or cryptate, and one or more acceptor labels comprising a fluorescent or chemiluminescent dye, wherein the excitation and emission spectra of the acceptor label are compatible with the donor label (in a time-resolved fluorescence resonance energy transfer (TR-FRET) reaction and/or a time-resolved amplified cryptate emission (TRACE) reaction, preferably according to claim 7),
wherein said labels are in physical proximity to said antibodies and/or are conjugated to said antibodies in said kit.

In one embodiment, the kit comprises:
a. detection reagents for determining the level of proADM or fragment(s) thereof in a sample from a patient, such as the antibodies described herein, and
b. reference data with regard to any relevant disease associated with MR-proADM, or for which MR-proADM may be used as a diagnostic, prognostic or therapy guidance marker, in particular a reference, threshold or cut-off value, wherein said reference data is preferably stored on a computer readable medium and/or employed in the form of computer executable code configured for comparing the determined levels of proADM or fragment(s) thereof with a cut-off value,
d. and optionally, detection reagents for determining the level of at least one additional parameter or biomarker or fragment(s) thereof, preferably PCT, D-Dimer, Troponin, IP10, IL6, body weight and/or age, in a sample from a patient, and reference data, such as reference levels for said at least one additional biomarker, preferably PCT, for a risk threshold or cut-off value, wherein said reference data is preferably stored on a computer readable medium and/or employed in the form of computer executable code, such as an algorithm, configured for comparing the determined levels of the at least one additional biomarker or fragment(s) thereof with the threshold or cut-off value.

Various clinical applications are possible with the present invention, for example, determination of, by way of non-limiting example, infection, adverse events, death, organ dysfunction or damage, diagnosis of the severity or prognosis of the risk of developing a severe health state of a patient; including both rule-in and rule out of medical conditions or determining a risk to a patient or a treatment efficacy (responder/non-responder).

Using the kit and method of the present invention, it is possible to base a decision about diagnosis, prognosis or therapy guidance for patients on objective criteria, such as the reference data of the kit.

The reference data of the kit can be provided on a computer executable code configured for comparing the determined levels of proADM or fragment(s) thereof with said reference range. In some embodiments, the features of the kit may be considered to include those features used in characterizing the methods of the invention, and vice versa.

Further additional markers that may be determined in the context of the present invention comprise lactate and CRP. Clinical scores that may be determined in the context of the present invention comprise SI score (systemic inflammation score), SOFA, qSOFA, SIRS, SAPS II, APACHE II, CRB-65, PSI.

Determining proADM or fragment(s) thereof, and at least one clinical score, such as SOFA, PSI or CRB-65, in the context of the method of the invention turned out to provide further accuracy with respect to prognosis of a subsequent condition requiring hospital admission.

In further embodiments, the at least one additional biomarker is at least one marker of PCT, lactate, rhabdomyolysis, such as creatine kinase (CK), lactate dehydrogenase (LDH), creatinine, myoglobin, aldolase, troponin, carbonic anhydrase type 3, fatty acid-binding protein (FABP), transaminases or potassium.

The detection reagents (antibodies) for determining the level of proADM or fragment(s) thereof, and optionally for determining the level of PCT, lactate and/or C-reactive protein or fragment(s) thereof, are preferably selected from those necessary to perform the method, for example antibodies directed to MR-proADM, suitable labels, such as fluorescent labels, preferably two separate fluorescent labels suitable for application in the KRYPTOR assay, sample collection tubes.

The embodiments and features disclosed in the context of the methods of the invention also apply to the kit of the present invention and vice versa.

In one embodiment of the method described herein, the method additionally comprises comparing the determined level of proADM or fragment(s) thereof to a reference level, threshold value and/or a population average corresponding to proADM or fragments thereof in patients, wherein said comparing is carried out in a computer processor using computer executable code.

The methods of the present invention may in part be computer-implemented. For example, the step of comparing the detected level of a marker, e.g. the proADM or fragments thereof, with a reference level can be performed in a computer system. In the computer-system, the determined level of the marker(s) can be combined with other marker levels and/or parameters of the subject in order to calculate a score, which is indicative for the diagnosis, prognosis, prediction, risk assessment and/or risk stratification. For example, the determined values may be entered (either manually by a health professional or automatically from the device(s) in which the respective marker level(s) has/have been determined) into the computer-system. The computer-system can be directly at the point-of-care (e.g. primary care, ICU or ED) or it can be at a remote location connected via a computer network (e.g. via the internet, or specialized medical cloud-systems, optionally combinable with other IT-systems or platforms such as hospital information systems (HIS)). Typically, the computer-system will store the values (e.g. marker level or parameters such as age, blood pressure, weight, sex, etc. or clinical scoring systems such as SOFA, qSOFA, BMI etc.) on a computer-readable medium and calculate the score based-on pre-defined and/or pre-stored reference levels or reference values. The resulting score will be displayed and/or printed for the user (typically a health professional such as a physician). Alternatively or in addition, the associated prognosis, diagnosis, assessment, treatment guidance, patient management guidance or stratification will be displayed and/or printed for the user (typically a health professional such as a physician).

In one embodiment of the invention, a software system can be employed, in which a machine learning algorithm is evident, preferably to identify hospitalized patients at risk for sepsis, severe sepsis and septic shock using data from electronic health records (EHRs). A machine learning approach can be trained on a random forest classifier using EHR data (such as labs, biomarker expression, vitals, and demographics) from patients. Machine learning is a type of artificial intelligence that provides computers with the ability to learn complex patterns in data without being explicitly programmed, unlike simpler rule-based systems. Earlier studies have used electronic health record data to trigger alerts to detect clinical deterioration in general. In one embodiment of the invention the processing of proADM levels may be incorporated into appropriate software for comparison to existing data sets, for example proADM levels may also be processed in machine learning software to assist in diagnosing or prognosing the occurrence of an adverse event.

The combined employment of MR-proADM antibodies in combination with another biomarker assay such as for PCT or CRP may be realized either in a single multiplex assay, or in two separate assays conducted on a sample form the patient. The sample may relate to the same sample, or to different samples. The assay employed for the detection and determination of MR-proADM and for example PCT may also be the same or different, for example an immunoassay may be employed for the determination of one of the above markers. More detailed descriptions of suitable assays are provided below.

Additionally, functional assay sensitivity can be determined in order to indicate statistically significant values for use as reference levels or cut-offs according to established techniques. Laboratories are capable of independently establishing an assay's functional sensitivity by a clinically relevant protocol. “Functional sensitivity” can be considered as the concentration that results in a coefficient of variation (CV) of 20% (or some other predetermined % CV, or inter-assay precision), and is thus a measure of an assay's precision at low analyte levels. The CV (or functional assay sensitivity) is therefore a standardization of the standard deviation (SD) that allows comparison of variability estimates regardless of the magnitude of analyte concentration, at least throughout most of the working range of the assay.

Furthermore, methods based on ROC analysis can be used to determine statistically significant differences between two clinical patient groups. Receiver Operating Characteristic (ROC) curves measure the sorting efficiency of the model's fitted probabilities to sort the response levels. ROC curves can also aid in setting criterion points in diagnostic tests. The higher the curve from the diagonal, the better the fit. If the logistic fit has more than two response levels, it produces a generalized ROC curve. In such a plot, there is a curve for each response level, which is the ROC curve of that level versus all other levels. Software capable of enabling this kind of analysis in order to establish suitable reference levels and cut-offs is available, for example JMP 12, JMP 13, Statistical Discovery, from SAS.

Cut off values may similarly be determined for any other biomarker used in combination with MR-proADM. Literature is available to a skilled person for determining an appropriate cut-off, for example Philipp Schuetz et al. (BMC Medicine. 2011; 9:107) describe that at a cut-off of 0.1 ng/mL, PCT had a very high sensitivity to exclude infection. Terence Chan et al. (Expert Rev.Mol.Diagn.2011; 11(5), 487.496) described that indicators such as the positive and negative likelihood ratios, which are calculated based on sensitivity and specificity, are also useful for assessing the strength of a diagnostic test. Values are commonly graphed for multiple cut-off values (CVs) as a receiver operating characteristic curve. The area under the curve value is used to determine the best diagnostically relevant CV. This literature describes the variation of CVs (cut-off values, that is dependent on the assay and study design), and suitable methods for determining cut-off values.

Population averages levels of MR-proADM or fragments thereof may also be used as reference values, for example mean MR-proADM population values, whereby patient samples may be compared to a control population, wherein the control group preferably comprises more than 10, 20, 30, 40, 50 or more subjects.

In one embodiment of the invention, a cut-off level for MR-proADM—for example for any given one or more diagnostic, prognostic or therapy guidance statements—may be a value in the range of 0.005 to 50.00 nmol/L in a plasma sample. In some embodiments, the cut-off level is in the range of 0.01 to 30, 0.05 to 20, 0.1 to 15, or 0.1 to 10 ng/mL. Any value within these ranges may be considered as an appropriate cut-off value.

In one embodiment of the invention, combined PCT determination may be employed in parallel or in combination with the inventive assay.

In embodiments, the cut-off level for PCT may be a value in the range of 0.01 to 100.00 ng/mL in a serum sample, when using for example a Luminex MAC Pix E-Bioscience Assay or the B⋅R⋅A⋅H⋅M⋅S PCT-Kryptor Assay. In a preferred embodiment the cut-off level of PCT may be in the range of 0.01 to 100, 0.05 to 50, 0.1 to 20, or 0.1 to 2 ng/mL, and most preferably >0.05 to 0.5 ng/mL. Any value within these ranges may be considered as an appropriate cut-off value. For example, 0.01, 0.05, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 3, 4, 5, 6, 7, 8, 9, 10, 15, 20, 25, 30, 35, 40, 45, 50, 55, 60, 65, 70, 75, 80, 85, 90, 95 or 100 ng/mL may be employed. In some embodiments, PCT levels for healthy subjects are approximately 0.05 ng/mL.

### Embodiments Related to the Antibodies of the Invention and Uses Thereof

In light of the prior art, another technical problem underlying the invention was the provision of an alternative or improved binding agent, suitable as a diagnostic, therapeutic and/or prophylactic reagent for identifying, determining and/or measuring MR-proADM and/or diagnosing and/or treating medical conditions associated with MR-proADM.

A further object of the invention was the provision of an antibody or antibody fragment, with improved properties.

A further object of the invention was the provision of an antibody or antibody fragment, with improved affinity to a MR-proADM epitope.

A further object of the invention was the provision of an antibody or antibody fragment, with improved sensitivity in detecting MR-proADM.

A further object of the invention was the provision of an antibody or antibody fragment, with reduced unwanted, unspecific cross-reactions with off-target proteins.

The invention therefore further relates to a monoclonal antibody or antibody fragment that binds mid-regional pro-adrenomedullin (MR-proADM). In embodiments, the antibodies described herein may be employed in the inventive methods, kits and in other aspects of the invention.

In embodiments, the antibody of fragment thereof is a murine antibody, preferably a mouse antibody, for example wherein the antibody of fragment thereof comprises or consists of an amino acid sequence isolated after immunization of a murine subject.

In embodiments, the antibody or fragment thereof binds an MR-proADM epitope within the sequence according to SEQ ID NO 3.

In embodiments, the antibody or fragment thereof binds an MR-proADM epitope according to one or more of SEQ ID NO 5-18.

In one embodiment, the antibody “Ab1” binds an epitope according to SEQ ID NO 5 or 6.

In one embodiment, the antibody “Ab2” binds an epitope according to SEQ ID NO 7 or 8.

In one embodiment, the antibody “Ab3” binds an epitope according to SEQ ID NO 9 or 10.

In one embodiment, the antibody “Ab4” binds an epitope according to SEQ ID NO 11 or 12.

In one embodiment, the antibody “Ab5” binds an epitope according to SEQ ID NO 13 or 14.

In one embodiment, the antibody “Ab6” binds an epitope according to SEQ ID NO 15 or 16.

In one embodiment, the antibody “Ab7” binds an epitope according to SEQ ID NO 17 or 18.

In one embodiment, the antibody or antibody fragment is defined by one or more complementary determining regions (CDRs) as described herein, preferably by 6 CDR regions, 3 from the variable region of the heavy chain, and 3 from the variable region of the light chain. The CDRs described herein may be used in the specific combination as discovered, or in any given combination for which the binding to the target protein is maintained.

In embodiments, the antibody comprises:
a heavy chain (variable VH) domain, said VH domain comprising complementary determining region (CDR) sequences of:
   H-CDR1 selected from SEQ ID NO 138, 139, 153, 154, 168, 169, 183, 184, 198, 199, 213, 214, 228 or 229, or a CDR sequence of at least 80%, preferably 90%, identity to any one of the aforementioned CDR sequences,
   H-CDR2 selected from SEQ ID NO 140, 141, 155, 156, 170, 171, 185, 186, 200, 201, 215, 216, 230 or 231, or a CDR sequence of at least 80%, preferably 90%, identity to any one of the aforementioned CDR sequences, and
   H-CDR3 selected from SEQ ID NO 142, 143, 157, 158, 172, 173, 187, 188, 202, 203, 217, 218, 232 or 233, or a CDR sequence of at least 80%, preferably 90%, identity to any one of the aforementioned CDR sequences,
and a light chain variable (VL) domain, said VL domain comprising complementary determining region (CDR) sequences of:
   L-CDR1 selected from SEQ ID NO 144, 145, 159, 160, 174, 175, 189, 190, 204, 205, 219, 220, 234 or 235, or a CDR sequence of at least 80%, preferably 90%, identity to any one of the aforementioned CDR sequences,
   L-CDR2 selected from SEQ ID NO 146, 147, 161, 162, 176, 177, 191, 192, 206, 207, 221, 222, 236 or 237, or a CDR sequence of at least 80%, preferably 90%, identity to any one of the aforementioned CDR sequences, and
   L-CDR3 selected from SEQ ID NO 148, 163, 178, 193, 208, 223, or 238, or a CDR sequence of at least 80%, preferably 90%, identity to any one of the aforementioned CDR sequences.

In embodiments, the antibody comprises:
a heavy chain (variable VH) domain, said VH domain comprising complementary determining region (CDR) sequences of:
   H-CDR1 selected from SEQ ID NO 138, 153, 168, 183, 198, 213, or 228,
   H-CDR2 selected from SEQ ID NO 140, 155, 170, 185, 200, 215, or 230,
   H-CDR3 selected from SEQ ID NO 142, 157, 172, 187, 202, 217, or 232,
and a light chain variable (VL) domain, said VL domain comprising complementary determining region (CDR) sequences of:
   L-CDR1 selected from SEQ ID NO 144, 159, 174, 189, 204, 219, or 234,
   L-CDR2 selected from SEQ ID NO 146, 161, 176, 191, 206, 221, or 236,
   L-CDR3 selected from SEQ ID NO 148, 163, 178, 193, 208, 223, or 238.

In embodiments, the antibody comprises:
a heavy chain (variable VH) domain, said VH domain comprising complementary determining region (CDR) sequences of:
   H-CDR1 selected from SEQ ID NO 139, 154, 169, 184, 199, 214, or 229,
   H-CDR2 selected from SEQ ID NO 141, 156, 171, 186, 201, 216, or 231,
   H-CDR3 selected from SEQ ID NO 143, 158, 173, 188, 203, 218, or 233,
and a light chain variable (VL) domain, said VL domain comprising complementary determining region (CDR) sequences of:
   L-CDR1 selected from SEQ ID NO 145, 160, 175, 190, 205, 220, or 235,
   L-CDR2 selected from SEQ ID NO 147, 162, 177, 192, 207, 222, or 237,
   L-CDR3 selected from SEQ ID NO 148, 163, 178, 193, 208, 223, or 238.

In embodiments, the antibody or antibody fragment of the invention comprises a heavy chain variable (VH) domain, said VH domain comprising a sequence of at least 70% identity, preferably 80% identity, more preferably 90% identity, to one of SEQ ID NO 136, 151, 166, 181, 196, 211 or 226, and a light chain variable (VL) domain, said VL domain comprising a sequence of at least 70% identity, preferably 80% identity, more preferably 90% identity, to one of SEQ ID NO 137, 152, 167, 182, 197, 212 or 227.

In embodiments, the antibody or antibody fragment of the invention comprises a heavy chain variable (VH) domain and a light chain variable (VL) domain, said VH and VL comprising CDR sequences according to:
H-CDR1 according to SEQ ID NO 138 or 139,
H-CDR2 according to SEQ ID NO 140 or 141,
H-CDR3 according to SEQ ID NO 142 or 143,
L-CDR1 according to SEQ ID NO 144 or 145,
L-CDR2 according to SEQ ID NO 146 or 147, and
L-CDR3 according to SEQ ID NO 148, (Ab1) or
H-CDR1 according to SEQ ID NO 153 or 154,
H-CDR2 according to SEQ ID NO 155 or 156,
H-CDR3 according to SEQ ID NO 157 or 158,
L-CDR1 according to SEQ ID NO 159 or 160,
L-CDR2 according to SEQ ID NO 161 or 162, and
L-CDR3 according to SEQ ID NO 163, (Ab2) or
H-CDR1 according to SEQ ID NO 168 or 169,
H-CDR2 according to SEQ ID NO 170 or 171,
H-CDR3 according to SEQ ID NO 172 or 173,
L-CDR1 according to SEQ ID NO 174 or 175,
L-CDR2 according to SEQ ID NO 176 or 177, and
L-CDR3 according to SEQ ID NO 178, (Ab3) or
H-CDR1 according to SEQ ID NO 183 or 184,
H-CDR2 according to SEQ ID NO 185 or 186,
H-CDR3 according to SEQ ID NO 187 or 188,
L-CDR1 according to SEQ ID NO 189 or 190,
L-CDR2 according to SEQ ID NO 191 or 192, and
L-CDR3 according to SEQ ID NO 193, (Ab4) or
H-CDR1 according to SEQ ID NO 198 or 199,
H-CDR2 according to SEQ ID NO 200 or 201,
H-CDR3 according to SEQ ID NO 202 or 203,
L-CDR1 according to SEQ ID NO 204 or 205,
L-CDR2 according to SEQ ID NO 206 or 207, and
L-CDR3 according to SEQ ID NO 208, (Ab5) or
H-CDR1 according to SEQ ID NO 213 or 214,
H-CDR2 according to SEQ ID NO 215 or 216,
H-CDR3 according to SEQ ID NO 217 or 218,
L-CDR1 according to SEQ ID NO 219 or 220,
L-CDR2 according to SEQ ID NO 221 or 222, and
L-CDR3 according to SEQ ID NO 223, (Ab6) or
H-CDR1 according to SEQ ID NO 228 or 229,
H-CDR2 according to SEQ ID NO 230 or 231,
H-CDR3 according to SEQ ID NO 232 or 233,
L-CDR1 according to SEQ ID NO 234 or 235,
L-CDR2 according to SEQ ID NO 236 or 237, and
L-CDR3 according to SEQ ID NO 238, (Ab7)

In embodiments, at any given CDR, the first listed CDR sequence above may be combined with the other first listed CDR sequences. In embodiments, at any given CDR, the second listed CDR sequence above may be combined with the other second listed CDR sequences.

In embodiments, the antibody or antibody fragment of the invention comprises VH and VL domains that comprise the sequences according to SEQ ID NO 136 and 137 (Ab1), 151 and 152 (Ab2), 166 and 167 (Ab3), 181 and 182 (Ab4), 196 and 197 (Ab5), 211 and 212 (Ab6), or 226 and 227 (Ab7), respectively.

In embodiments, the CDR sequences presented above are shown with alternative sequences, where each CDR may be selected from 1, 2 or 3 similar sequences. In embodiments, these alternatives result from an alternative annotation of the variable sequences to find alternative definitions of CDR sequences, for example using IMGT or Kabat CDR prediction models. In embodiments, a combination of any 6 CDRs, including at least one of each H-CDRs 1-3 and L-CDRs 1-3, from any given selection or combination of CDR annotation model is covered by the present invention and intended as disclosed in the present application.

### Embodiments for Alternative Antibody Sequences

In embodiments, the antibody comprises:
a heavy chain (variable VH) domain, said VH domain comprising complementary determining region (CDR) sequences of:
   H-CDR1 selected from SEQ ID NO 23, 24, 25, 41, 42, 56, 57, 58, 70, 71, 86, 87, 88, 104, 105, 121, 122, or 123, or a CDR sequence of at least 80%, preferably 90%, identity to any one of the aforementioned CDR sequences,
   H-CDR2 selected from SEQ ID NO 26, 27, 28, 43, 44, 59, 72, 73, 74, 89, 90, 91, 106, 107, 124, 125 or 126, or a CDR sequence of at least 80%, preferably 90%, identity to any one of the aforementioned CDR sequences,
   H-CDR3 selected from SEQ ID NO 29, 30, 31, 45, 60, 75, 92, 93, 108, 109, 110, 127 or 128, or a CDR sequence of at least 80%, preferably 90%, identity to any one of the aforementioned CDR sequences,
and a light chain variable (VL) domain, said VL domain comprising complementary determining region (CDR) sequences of:
   a. L-CDR1 selected from SEQ ID NO 32, 33, 46, 47, 61, 62, 76, 77, 94, 95, 111, 112, 129 or 130, or a CDR sequence of at least 80%, preferably 90%, identity to any one of the aforementioned CDR sequences,
   b. L-CDR2 selected from SEQ ID NO 34, 35, 48, 49, 63, 64, 78, 79, 80, 96, 97, 113, 114, 131, 132, or a CDR sequence of at least 80%, preferably 90%, identity to any one of the aforementioned CDR sequences,
   c. L-CDR3 selected from SEQ ID NO 36, 50, 51, 65, 81, 98, 99, 115, 116 or 133, or a CDR sequence of at least 80%, preferably 90%, identity to any one of the aforementioned CDR sequences.

In embodiments, the antibody comprises:
a heavy chain (variable VH) domain, said VH domain comprising complementary determining region (CDR) sequences of:
   H-CDR1 selected from SEQ ID NO 23, 24, 25, 41, 42, 56, 57, 58, 70, 71, 86, 87, 88, 104, 105, 121, 122, or 123,
   H-CDR2 selected from SEQ ID NO 26, 27, 28, 43, 44, 59, 60, 72, 73, 74, 89, 90, 91, 106, 107, 124, 125 or 126,
   H-CDR3 selected from SEQ ID NO 29, 30, 31, 45, 60, 75, 92, 93, 108, 109, 110, 127 or 128,
and a light chain variable (VL) domain, said VL domain comprising complementary determining region (CDR) sequences of:
   d. L-CDR1 selected from SEQ ID NO 32, 33, 46, 47, 61, 62, 76, 77, 78, 94, 95, 111, 112, 129 or 130,
   e. L-CDR2 selected from SEQ ID NO 34, 35, 48, 49, 63, 64, 78, 79, 80, 96, 97, 113, 114, 131, 132,
   f. L-CDR3 selected from SEQ ID NO 36, 50, 51, 65, 81, 98, 99, 115, 116 or 133.

In embodiments, the antibody or antibody fragment of the invention comprises a heavy chain variable (VH) domain, said VH domain comprising a sequence of at least 70% identity to one of SEQ ID NO 21, 39, 54, 68, 84, 102, or 119, and a light chain variable (VL) domain, said VL domain comprising a sequence of at least 70% identity to one of SEQ ID NO 22, 40, 55, 69, 85, 103, or 120.

In embodiments, the antibody or antibody fragment of the invention comprises a heavy chain variable (VH) domain and a light chain variable (VL) domain, said VH and VL comprising CDR sequences according to:
H-CDR1 according to SEQ ID NO 23, 24 or 25,
H-CDR2 according to SEQ ID NO 26, 27 or 28,
H-CDR3 according to SEQ ID NO 29, 30 or 31,
L-CDR1 according to SEQ ID NO 32 or 33,
L-CDR2 according to SEQ ID NO 34 or 35, and
L-CDR3 according to SEQ ID NO 36, (Alternative Ab1) or
H-CDR1 according to SEQ ID NO 41 or 42,
H-CDR2 according to SEQ ID NO 43 or 44,
H-CDR3 according to SEQ ID NO 45,
L-CDR1 according to SEQ ID NO 46 or 47,
L-CDR2 according to SEQ ID NO 48 or 49, and
L-CDR3 according to SEQ ID NO 50 or 51, (Alternative Ab2) or
H-CDR1 according to SEQ ID NO 56, 57, or 58,
H-CDR2 according to SEQ ID NO 59,
H-CDR3 according to SEQ ID NO 60,
L-CDR1 according to SEQ ID NO 61 or 62,
L-CDR2 according to SEQ ID NO 63 or 64, and
L-CDR3 according to SEQ ID NO 65, (Alternative Ab3) or
H-CDR1 according to SEQ ID NO 70 or 71,
H-CDR2 according to SEQ ID NO 72, 73, or 74,
H-CDR3 according to SEQ ID NO 75,
L-CDR1 according to SEQ ID NO 76 or 77,
L-CDR2 according to SEQ ID NO 78, 79, or 80, and
L-CDR3 according to SEQ ID NO 81, (Alternative Ab4) or
H-CDR1 according to SEQ ID NO 86, 87 or 88,
H-CDR2 according to SEQ ID NO 89, 90 or 91,
H-CDR3 according to SEQ ID NO 92 or 93,
L-CDR1 according to SEQ ID NO 94 or 95,
L-CDR2 according to SEQ ID NO 96 or 97, and
L-CDR3 according to SEQ ID NO 98 or 99, (Alternative Ab5) or
H-CDR1 according to SEQ ID NO 104 or 105,
H-CDR2 according to SEQ ID NO 106 or 107,
H-CDR3 according to SEQ ID NO 108, 109 or 110,
L-CDR1 according to SEQ ID NO 111 or 112,
L-CDR2 according to SEQ ID NO 113 or 114, and
L-CDR3 according to SEQ ID NO 115 or 116, (Alternative Ab6) or
H-CDR1 according to SEQ ID NO 121, 122 or 123,
H-CDR2 according to SEQ ID NO 124, 125 or 126,
H-CDR3 according to SEQ ID NO 127 or 128,
L-CDR1 according to SEQ ID NO 129 or 130,
L-CDR2 according to SEQ ID NO 131 or 132, and
L-CDR3 according to SEQ ID NO 133 (Alternative Ab7).

In embodiments, the antibody or antibody fragment of the invention comprises VH and VL domains that comprise the sequences according to SEQ ID NO 21 and 22, 39 and 40, 54 and 55, 68 and 69, 84 and 85, 102 and 103, or 119 and 120, respectively.

### Further Embodiments

With respect to the CDR sequences and sequence variation with any given sequence identity to the specific sequence, a skilled person is capable of determining antibody binding and can obtain, without undue effort, a set of CDR sequences with the given sequence identity that maintain the binding properties of the antibodies disclosed herein.

Should the sequence be of a length, that cannot plausibly have sequence variation that would lead to an e.g. 90% sequence identity, a skilled person is also capable of determining such sequences and adjusting or excluding the sequences appropriately. Considering the detailed information presented in the examples regarding the desired binding characteristics of the antibodies, any given antibody with sufficient sequence identity can be determined without undue effort.

Regarding the L-CDR2 sequences, these comprise or consist of, in some embodiments, three amino acids. In some embodiments, amino acid sequence variation is possible, such that e.g. 1 of the 3 amino acids is changed from the original sequence and binding is maintained. Such variants, with ⅔ amino acid identities to the original sequences, especially those with conserved substitutions, are encompassed in the scope of the invention. The specific L-CDR2 sequences are provided below.

In one embodiment, the VH and VL combinations are as described in the antibodies as originally isolated.

In one embodiment, the antibody or antibody fragment comprises VH and VL domains with the specific CDR sequences as described in the antibodies as isolated, and additionally are characterized by a framework sequence with sequence similarity as described herein to the specific VH and VL sequences as isolated.

For example, the CDR sequences are those as specified in the isolated antibodies, and outside the specified CDR sequences, the antibodies comprise adjacent framework sequences with:
an at least 70%, preferably at least 80% or at least 90%, or more preferably at least 95% or complete sequence identity to the relevant non-CDR portion of a variable domain, as described herein, for example to VH and VL sequences of SEQ ID NO 136 and 137 (Ab1), 151 and 152 (Ab2), 166 and 167 (Ab3), 181 and 182 (Ab4), 196 and 197 (Ab5), 211 and 212 (Ab6), or 226 and 227 (Ab7), respectively.

For example, using alternative sequences, the CDR sequences are those as specified in the isolated antibodies, and outside the specified CDR sequences, the antibodies comprise adjacent framework sequences with:
an at least 70%, preferably at least 80% or at least 90%, or more preferably at least 95% or complete sequence identity to the relevant non-CDR portion of a variable domain, as described herein, for example to VH and VL sequences of SEQ ID NO 21 and 22, 39 and 40, 54 and 55, 68 and 69, 84 and 85, 102 and 103, or 119 and 120, respectively.

In embodiments of the invention, the amino acid sequence variation used to obtain said antibodies may occur in either the CDR regions of the original antibody or in the framework regions, wherein the framework region is to be understood as a region in the variable domain of a protein which belongs to the immunoglobulin superfamily, and which is less “variable” than the CDRs.

In preferred embodiments, any sequence variant that maintains any one or more of the binding properties disclosed herein is encompassed by the invention.

In one embodiment, Ab1 may be defined by one or more, preferably a combination of all 6, CDR sequences according to the sequences, sequence alignments, or sequence formulae below:

H-CDR1:
EYTIH
GYTFTEYT
GYTFTEYTLH
SGYTFTEYTLHW
GYTFTEYT
(SEQ ID NO 239)
XGYTFTEYTXXX
(SEQ ID NO 240)
GYTFTEYT
H-CDR2:
SIIPKNAFTRYNQKFKG
IIPKNAFT
SLLPKNNSSPPRSGG
GSLLPKNNSSPPRSGGK
LLPKNN
(SEQ ID NO 241)
XXXXPKNXXXXXXXXXX
(SEQ ID NO 242)
XXPKNX
H-CDR3:
PGYDGHIWFPN
ARPGYDGHIWFPN
TGDQGTSHHMRSPMHWFPN
TTGDQGTSHHMRSPMHWFPNW
MGAATTGDQGTSHHMRSPMHWFPN
(SEQ ID NO 243)
XXXXXXXXXXXXXXXXXXXXWFPNX
(SEQ ID NO 244)
XXXXXXXWFPN
L-CDR1:
RSSQSIVHSNGNTYLE
QSIVHSNGNTY
NSSQSIVHSNGNTYLE
QSIVHSNGNTY
(SEQ ID NO 245)
XXXQSIVHSNGNTYXX
L-CDR2:
KVS
KVSNRFS
KVS
(SEQ ID NO 246)
KVSXXXX
L-CDR3:
FQGSHVPWT
DQGSHVPWT
(SEQ ID NO 247)
XQGSHVPWT

In the embodiments and alignments above, each X (considered a variable position in the sequence) may be any amino acid, or preferably the amino acids listed at each variable position (when reading the alignment at each position “vertically”), or preferably any conservative amino acid substitution (as disclosed by way of example below) at each variable position. In embodiments, any given CDR sequence may be defined only by the amino acids for which no variation is observed across the alignment. In embodiments, this may relate to the general sequences at the bottom of each alignment, without any X residues, i.e. only the amino acids that do not vary across the sequences in the alignment may be used to define the CDR.

In one embodiment, Ab2 may be defined by one or more, preferably a combination of all 6, CDR sequences according to the sequences, sequence alignments, or sequence formulae below:

H-CDR1:
DYSMH
GYTFTDY
GYTFTDYSMH
GYTFTDYS
(SEQ ID NO 248)
GYTFTDYXXX
H-CDR2:
INTETGEP
WINTETGEPTYVDDFKG
INTETGEP
(SEQ ID NO 249)
XINTETGEPXXXXXXXX
H-CDR3:
NFDY
ATNFDY
QHQHHNFKDVLEGNEQF
(SEQ ID NO 250)
XXXXXNFXXXXXXXXXX
L-CDR1:
QSLLDSDGQTY
KSSQSLLDSDGQTYLH
QSLLDSDGQTY
(SEQ ID NO 251)
XXXQSLLDSDGQTYXX
L-CDR2:
LVS
LVSKLGS
LVS
(SEQ ID NO 252)
LVSXXXX
L-CDR3:
WQGTHFPRT
SGGEGAPDL
SSGGEGAPDL
(SEQ ID NO 253)
XXXGXXXPXX

In the embodiments and alignments above, each X (considered a variable position in the sequence) may be any amino acid, or preferably the amino acids listed at each variable position (when reading the alignment at each position “vertically”), or preferably any conservative amino acid substitution (as disclosed by way of example below) at each variable position. In embodiments, any given CDR sequence may be defined only by the amino acids for which no variation is observed across the alignment. In embodiments, this may relate to the general sequences at the bottom of each alignment, without any X residues, i.e. only the amino acids that do not vary across the sequences in the alignment may be used to define the CDR.

In one embodiment, Ab3 may be defined by one or more, preferably a combination of all 6, CDR sequences according to the sequences, sequence alignments, or sequence formulae below:

H-CDR1:
DYFMH
(SEQ ID NO 250)
GYSFTDYF
LNLPDKDQLQALEK
NLPDKDQLQALE
PDKDQLQA
(SEQ ID NO 254)
XXXXDXXXXXXXXX
H-CDR2:
RLNPYNGATNYNQNFKD
LNPYNGAT
(SEQ ID NO 255)
XLNPYNGATXXXXXXXX
H-CDR3:
FTTPPEAY
LSFTTPPEAY
HDGTRSFTTPPEAY
(SEQ ID NO 256)
XXXXXXFTTPPEAY
L-CDR1:
RSNKSLLYSNGITYLF
HSSKSLLYSNGLTYLF
KSLLYSNGLTY
KSLLYSNGITY
(SEQ ID NO 257)
XXXKSLLYSNGXTYXX
L-CDR2:
QMSNLAS
QMSNLAS
QMS
(SEQ ID NO 258)
QMSXXXX
L-CDR3:
AQNLELPWT
AQNLELPWT
(SEQ ID NO 259)
XAQNLELPWTX

In the embodiments and alignments above, each X (considered a variable position in the sequence) may be any amino acid, or preferably the amino acids listed at each variable position (when reading the alignment at each position “vertically”), or preferably any conservative amino acid substitution (as disclosed by way of example below) at each variable position. In embodiments, any given CDR sequence may be defined only by the amino acids for which no variation is observed across the alignment. In embodiments, this may relate to the general sequences at the bottom of each alignment, without any X residues, i.e. only the amino acids that do not vary across the sequences in the alignment may be used to define the CDR.

In one embodiment, Ab4 may be defined by one or more, preferably a combination of all 6, CDR sequences according to the sequences, sequence alignments, or sequence formulae below:

H-CDR1:
EYTMH
GYTFTEYT
GSTFTEYTMH
GSTFTEYT
(SEQ ID NO 260)
XXXXXEYTXX
H-CDR2:
GINPNSGNSRFSQKFKD
GLNPNSGNSRFSAGKFKD
LNPNSGNSRFSAGKFKD
LNPNSGNSRFSAGKFKDRATLTVDKSSTT
INPNSGNS
(SEQ ID NO 261)
XXNPNSGNSRFSXXXXXXXXXXXXXXXXXX
(SEQ ID NO 262)
NPNSGNSRFS
H-CDR3:
YGYFAMDY
NYGDYGYFAMDY
TRNYGDYGYFAMDY
THHHNYGDYGYYAMDY
(SEQ ID NO 263)
XXXXNYGDYGYXAMDY
(SEQ ID NO 264)
YGYXAMDY
L-CDR1:
KASQDIHRYLSWFQQKP
KASQDIHRYLS
KASQDIHRYLS
QDIHRY
(SEQ ID NO 265)
XXXQDIHRYXX
(SEQ ID NO 266)
KASQDIHRYLSXXXXXX
L-CDR2:
RANSLVD
RANSLVD
ANSLVD
RAN
(SEQ ID NO 267)
XANXXXX
L-CDR3:
LQFAEFPLT
QFAEFPLT
(SEQ ID NO 268)
XQFAEFPLT

In the embodiments and alignments above, each X (considered a variable position in the sequence) may be any amino acid, or preferably the amino acids listed at each variable position (when reading the alignment at each position “vertically”), or preferably any conservative amino acid substitution (as disclosed by way of example below) at each variable position. In embodiments, any given CDR sequence may be defined only by the amino acids for which no variation is observed across the alignment. In embodiments, this may relate to the general sequences at the bottom of each alignment, without any X residues, i.e. only the amino acids that do not vary across the sequences in the alignment may be used to define the CDR.

In one embodiment, Ab5 may be defined by one or more, preferably a combination of all 6, CDR sequences according to the sequences, sequence alignments, or sequence formulae below:

H-CDR1:
GYSFTDPGDG
YSFTDPGD
SGYSFTDPGDGM
DYYMH
GYSFTDYY
(SEQ ID NO 269)
XXXXXXDXXXXX
(SEQ ID NO 270)
YSFTD
H-CDR2:
INPYNQT
NPYNQ
RINPYNQTK
RINPYNGATKYNQNFKD
INPYNGAT
(SEQ ID NO 271)
XXNPYNXXXXXXXXXXX
(SEQ ID NO 272)
INPYN
H-CDR3:
KMNSDGDY
WKMNSDGDY
RYDNDDGD
TRRYDNDDGDY
(SEQ ID NO 273)
XXXXXNXDGDX
(SEQ ID NO 274)
NXDGD
L-CDR1:
EASKSLLYSNGITYLY
RSSKSLLYSNGITYLY
KSLLYSNGITY
(SEQ ID NO 275)
XXXKSLLYSNGITYXX
(SEQ ID NO 276)
KSLLYSNGITY
L-CDR2:
QMSNLAS
QMS
(SEQ ID NO 277)
QMSXXXX
L-CDR3:
AQNLELPWT
(SEQ ID NO 278)
XAQNLELPWTX

In the embodiments and alignments above, each X (considered a variable position in the sequence) may be any amino acid, or preferably the amino acids listed at each variable position (when reading the alignment at each position “vertically”), or preferably any conservative amino acid substitution (as disclosed by way of example below) at each variable position. In embodiments, any given CDR sequence may be defined only by the amino acids for which no variation is observed across the alignment. In embodiments, this may relate to the general sequences at the bottom of each alignment, without any X residues, i.e. only the amino acids that do not vary across the sequences in the alignment may be used to define the CDR.

In one embodiment, Ab6 may be defined by one or more, preferably a combination of all 6, CDR sequences according to the sequences, sequence alignments, or sequence formulae below:

H-CDR1:
GYSFTDFSVH
GYSFTDFS
DFSVH
GYSFTDFS
(SEQ ID NO 279)
XXXXXDFSXX
H-CDR2:
WINTETGEPTYADDFKG
INTETGEP
(SEQ ID NO 280)
XINTETGEPXXXXXXXX
H-CDR3:
ETVLPKMRHNRYDDDY
VLPKMRHNRYDDDY
HMHSTASNVAPETVLPKMRHNRYDDDY
GGRYDDDY
ARGGRYDDDY
(SEQ ID NO 281)
XXGGRYDDDY
(SEQ ID NO 282)
XXXXRYDDDY
L-CDR1:
KSSQSLLNSGNGAKNYLA
QSLLNSGNGAKNY
KSSQSLLNSGNQKNYLA
QSLLNSGNQKNY
(SEQ ID NO 283)
XXXQSLLNSGNQKNYXX
(SEQ ID NO 284)
QSLLNSGN
L-CDR2:
GASTRQS
GAS
(SEQ ID NO 285)
GASXXXX
L-CDR3:
QNDHSYPLT
NDHSYPLT
(SEQ ID NO 286)
XXNDHSYPLTX

In the embodiments and alignments above, each X (considered a variable position in the sequence) may be any amino acid, or preferably the amino acids listed at each variable position (when reading the alignment at each position “vertically”), or preferably any conservative amino acid substitution (as disclosed by way of example below) at each variable position. In embodiments, any given CDR sequence may be defined only by the amino acids for which no variation is observed across the alignment. In embodiments, this may relate to the general sequences at the bottom of each alignment, without any X residues, i.e. only the amino acids that do not vary across the sequences in the alignment may be used to define the CDR.

In one embodiment, Ab7 may be defined by one or more, preferably a combination of all 6, CDR sequences according to the sequences, sequence alignments, or sequence formulae below:

H-CDR1:
GYSFTDFSVH
GYSFTDFSVHW
GYSFTDFS
DFSVH
GYSFTDFS
(SEQ ID NO 287)
XXXXXDFSXX
H-CDR2:
WINTETGEPTYADDFKGR
WINTETGEPTYADDFKG
INTETGEP
(SEQ ID NO 288)
XINTETGEPXXXXXXXX
H-CDR3:
EHMVTARNRYDDDY
KDFCREHMVTARNRYDDDY
GGRYDDDY
ARGGRYDDDY
(SEQ ID NO 289)
XXGGRYDDDY
(SEQ ID NO 290)
XXXYDDDY
L-CDR1:
KSSQSLLNSGNQKNYLA
QSLLNSGNQKNY
(SEQ ID NO 291)
XXXQSLLNSGNQKNYXX
L-CDR2:
GASTRQS
GAS
(SEQ ID NO 292)
GASXXXX
L-CDR3:
QNDHSYPLT
(SEQ ID NO 293)
XQNDHSYPLTX

In the embodiments and alignments above, each X (considered a variable position in the sequence) may be any amino acid, or preferably the amino acids listed at each variable position (when reading the alignment at each position “vertically”), or preferably any conservative amino acid substitution (as disclosed byway of example below) at each variable position. In embodiments, any given CDR sequence may be defined only by the amino acids for which no variation is observed across the alignment. In embodiments, this may relate to the general sequences at the bottom of each alignment, without any X residues, i.e. only the amino acids that do not vary across the sequences in the alignment may be used to define the CDR.

In embodiments, the antibody or antibody fragment of the invention comprises a heavy chain variable (VH) domain and a light chain variable (VL) domain, said VH and VL comprising CDR sequences according to:
H-CDR1 according to SEQ ID NO 239 or 240,
H-CDR2 according to SEQ ID NO 241 or 242,
H-CDR3 according to SEQ ID NO 243 or 244,
L-CDR1 according to SEQ ID NO 245,
L-CDR2 according to SEQ ID NO 246, and
L-CDR3 according to SEQ ID NO 247, (General Ab1)
   or
H-CDR1 according to SEQ ID NO 248,
H-CDR2 according to SEQ ID NO 249,
H-CDR3 according to SEQ ID NO 250,
L-CDR1 according to SEQ ID NO 251,
L-CDR2 according to SEQ ID NO 252, and
L-CDR3 according to SEQ ID NO 253, (General Ab2)
   or
H-CDR1 according to SEQ ID NO 254,
H-CDR2 according to SEQ ID NO 255,
H-CDR3 according to SEQ ID NO 256,
L-CDR1 according to SEQ ID NO 257,
L-CDR2 according to SEQ ID NO 258, and
L-CDR3 according to SEQ ID NO 259, (General Ab3) or
H-CDR1 according to SEQ ID NO 260,
H-CDR2 according to SEQ ID NO 261 or 262,
H-CDR3 according to SEQ ID NO 263 or 264,
L-CDR1 according to SEQ ID NO 265 or 266,
L-CDR2 according to SEQ ID NO 267, and
L-CDR3 according to SEQ ID NO 268, (General Ab4) or
H-CDR1 according to SEQ ID NO 269 or 270,
H-CDR2 according to SEQ ID NO 271 or 272,
H-CDR3 according to SEQ ID NO 273 or 274,
L-CDR1 according to SEQ ID NO 275 or 276,
L-CDR2 according to SEQ ID NO 277, and
L-CDR3 according to SEQ ID NO 278, (General Ab5) or
H-CDR1 according to SEQ ID NO 279,
H-CDR2 according to SEQ ID NO 280,
H-CDR3 according to SEQ ID NO 281 or 282,
L-CDR1 according to SEQ ID NO 283 or 284,
L-CDR2 according to SEQ ID NO 285, and
L-CDR3 according to SEQ ID NO 286, (General Ab6) or
H-CDR1 according to SEQ ID NO 287,
H-CDR2 according to SEQ ID NO 288,
H-CDR3 according to SEQ ID NO 289 or 290,
L-CDR1 according to SEQ ID NO 291,
L-CDR2 according to SEQ ID NO 292, and
L-CDR3 according to SEQ ID NO 293, (General Ab7).

In embodiments, the preferred CDR combinations are described in the table below, using Kabat CDR annotation:

| Ab | CDR Heavy chain (H-CDR 1-3) | | | CDR Light chain (L-CDR 1-3) | | |
|---|---|---|---|---|---|---|
| Ab | EYTI | SIIPKNAFTRYNQ | PGYDGHI | RSSQSIVHSNGN | KVSNR | FQGSHV |
| 1 | H | KFKG | WFPN | TYLE | FS | PWT |
| Ab | DYS | WINTETGEPTYV | NFDY | KSSQSLLDSDGQ | LVSKL | WQGTHF |
| 2 | MH | DDFKG | | TYLH | GS | PRT |
| Ab | DYF | RLNPYNGATNYN | FTTPPEAY | RSNKSLLYSNGIT | QMSN | AQNLELP |
| 3 | MH | QNFKD | | YLF | LAS | WT |
| Ab | EYT | GINPNSGNSRFS | YGYFAMD | KASQDIHRYLSW | RANSL | LQFAEFP |
| 4 | MH | QKFKD | Y | FQQKP | VD | LT |
| Ab | DYY | RINPYNGATKYN | RYDNDDG | RSSKSLLYSNGIT | QMSN | AQNLELP |
| 5 | MH | QNFKD | D | YLY | LAS | WT |
| Ab | DFS | WINTETGEPTYA | GGRYDDD | KSSQSLLNSGNQ | GASTR | QNDHSY |
| 6 | VH | DDFKG | Y | KNYLA | QS | PLT |
| Ab | DFS | WINTETGEPTYA | GGRYDDD | KSSQSLLNSGNQ | GASTR | QNDHSY |
| 7 | VH | DDFKG | Y | KNYLA | QS | PLT |

In embodiments, the preferred CDR combinations are described in the table below, using IMGT CDR annotation:

| Ab | CDR Heavy chain (H-CDR 1-3) | | | CDR Light chain (L-CDR 1-3) | | |
|---|---|---|---|---|---|---|
| Ab1 | GYTFTEYT | IIPKNAFT | ARPGYDGHIWFPN | QSIVHSNGNTY | KVS | FQGSHVPWT |
| Ab2 | GYTFTDY | INTETGEP | ATNFDY | QSLLDSDGQTY | LVS | WQGTHFPRT |
| Ab3 | GYSFTDYF | LNPYNGAT | LSFTTPPEAY | KSLLYSNGITY | QMS | AQNLELPWT |
| Ab4 | GYTFTEYT | INPNSGNS | TRNYGDYGYFAMDY | QDIHRY | RAN | LQFAEFPLT |
| Ab5 | GYSFTDYY | INPYNGAT | TRRYDNDDGDY | KSLLYSNGITY | QMS | AQNLELPWT |
| Ab6 | GYSFTDFS | INTETGEP | ARGGRYDDDY | QSLLNSGNQKNY | GAS | QNDHSYPLT |
| Ab7 | GYSFTDFS | INTETGEP | ARGGRYDDDY | QSLLNSGNQKNY | GAS | QNDHSYPLT |

It was entirely surprising that the particular antibodies provided herein, preferably defined by the CDR regions of the VL and VH regions involved in binding, or by the VH and VL sequences disclosed herein, exhibit the properties as demonstrated in the examples, and show binding characteristics that enable the desired therapeutic and/or diagnostic effect.

In general, any change to a CDR region made may also be considered as a feature of a CDR sequence when considered independently of the framework sequence as a whole. Such modified CDR sequences may be considered defining features of the present invention, either within or independent of their context in the entire framework region described herein. For example, the CDR sequences identified above may be considered a defining feature of the invention independently of the surrounding framework sequence. In some embodiments, sequence variation of VH and VL antibody sequences by way of percentage sequence identity can be employed, combined with specific CDR sequences.

All possible combinations of potential modifications for any given potentially variant residue proposed herein are encompassed by the present invention. By combining one or more of these substitutions, variants may be generated that exhibit the desired binding properties of the antibody originally isolated. The antibodies or parts thereof described herein also encompass a sequence with at least 70%, 75%, 80%, 85%, preferably 90%, or 95% sequence identity to those sequences disclosed explicitly.

As can be observed from the overall disclosure of the present invention, in embodiments, some variation in variable domain sequences, including variation in CDR sequences, is possible, based on various factors, for example the methods used to determine antibody sequence, the methodology for CDR annotation, as discussed in more detail below, or other factors. The various CDR sequences of the invention may therefore be combined by a skilled person without undue effort, without a loss or with no significant loss in the beneficial properties shown herein. Any CDR sequences from the sequence table may be combined to form a combination of any 6 CDRs, comprising H-CDR1-3 and L-CDR1-3.

A further aspect of the invention relates to a nucleic acid molecule comprising a nucleotide sequence that encodes an antibody or antibody fragment as described herein.

A further aspect of the invention relates to a host cell, such as a bacterial cell or mammalian cell, capable of producing an antibody or antibody fragment and/or comprising a nucleic acid molecule as described herein.

A further aspect of the invention relates to an analytical, detection and/or diagnostic kit or composition suitable for use in an analytical, detection and/or diagnostic assay, comprising the antibody or antibody fragment as described herein.

A further aspect of the invention relates to a pharmaceutical composition comprising the antibody or antibody fragment as described herein, with a pharmaceutically acceptable carrier.

In some embodiments, the antibody or antibody fragment of the invention exhibits a strong affinity to the MR-proADM target.

In some embodiments, Surface Plasmon Resonance (SPR) measurements can quantitatively determine the affinity of an antibody or fragment to its target, for example by using the K_{D} values determined using SPR.

In some embodiments, the antibody or antibody fragment has an affinity, preferably determined using K_{D} values, preferably determined using SPR, of <500 nM, preferably <100 nM, <50 nM, <10 nM, more preferably <5 nM, <2 nM, <1 nM, more preferably <0.1 nM, or <0.01 nM.

Expressed in other nomenclature, in some embodiments, the antibody or antibody fragment has an affinity, preferably determined using K_{D} values, preferably determined using SPR, of <5.0E-07, preferably <1.0E-07, <5.0E-08, <1.0E-08, more preferably <7.0E-09, <5.0E-09, <3.0E-09, <1.0E-09, or smaller.

A measurement of K_{D} represents the equilibrium dissociation constant, a ratio of koff/kon, between the antibody and its antigen. K_{D} and affinity are inversely related. The K_{D} value relates to the concentration of antibody (the amount of antibody needed for a particular experiment) and so the lower the K_{D} value (lower concentration) and thus the higher the affinity of the antibody. Example K_{D} values are:
K_{D} value Molar concentration (sensitivity)
E-04 to E-06 Micromolar (μM)
E-07 to E-09 Nanomolar (nM)

Antibodies with affinities represented by K_{D} values in the nanomolar range are especially beneficial any may not necessarily be expected upon monoclonal antibody production.

A further aspect of the invention relates to a nucleic acid molecule comprising a nucleotide sequence that encodes an antibody or antibody fragment as described herein.

In one embodiment, the invention relates to a preferably isolated nucleic acid molecule selected from the group consisting of:
a) a nucleic acid molecule comprising a nucleotide sequence
   which encodes an isolated antibody or antibody fragment as described herein,
   which encodes an amino acid sequence according to a heavy chain variable (VH) domain, said VH domain comprising a sequence of at least 70% identity, preferably 80% identity, more preferably 90% identity, to one of SEQ ID NO 136, 151, 166, 181, 196, 211 or 226, or which encodes an amino acid sequence according to a light chain variable (VL) domain, said VL domain comprising a sequence of at least 70% identity, preferably 80% identity, more preferably 90% identity, to one of SEQ ID NO 137, 152, 167, 182, 197, 212 or 227, or,
   when using alternative sequences, which encodes an amino acid sequence according to a heavy chain variable (VH) domain, said VH domain comprising a sequence of at least 70% identity, preferably 80% identity, more preferably 90% identity, to one of SEQ ID NO 21, 39, 54, 68, 84, 102, or 119, or encodes an amino acid sequence according to a light chain variable (VL) domain, said VL domain comprising a sequence of at least 70% identity, preferably 80% identity, more preferably 90% identity, to one of SEQ ID NO 22, 40, 55, 69, 85, 103, or 120, or
b) a nucleic acid molecule which is complementary to a nucleotide sequence in accordance with a);
c) a nucleic acid molecule comprising a nucleotide sequence having sufficient sequence identity to be functionally analogous/equivalent to a nucleotide sequence according to a) or b),
d) a nucleic acid molecule which, as a consequence of the genetic code, is degenerated into a nucleotide sequence according to a) through c); and
e) a nucleic acid molecule according to a nucleotide sequence of a) through d) which is modified by deletions, additions, substitutions, translocations, inversions and/or insertions and functionally analogous/equivalent to a nucleotide sequence according to a) through d).

A further aspect of the invention relates to a host cell, such as a bacterial cell or mammalian cell, capable of producing an antibody or antibody fragment and/or comprising a nucleic acid molecule as described herein.

A further aspect of the invention relates to a pharmaceutical composition comprising the isolated antibody or antibody fragment or a nucleic acid molecule or a host cell as described herein, with a pharmaceutically acceptable carrier.

The invention described herein comprises therefore the monoclonal production and functional characterization of monoclonal antibodies that bind MR-proADM. The properties of the antibodies and the corresponding sequences are described in detail herein. Surprisingly, the monoclonal antibodies described herein enable improvements in sensitivity in detecting MR-proADM in comparison to established immunoassays. As described in the examples below, when assessing plasma samples with fixed concentrations of target antigen, the antibodies described herein enabled greater signals to be obtained in comparison to the established approaches of the prior art. Furthermore, the present antibodies are highly specific and do not exhibit cross-reactivity to off-target proteins. The present antibodies also exhibit excellent and surprisingly high affinities when assessed with SPR, thus providing a further unexpected and beneficial property of the inventive antibodies.

### DETAILED DESCRIPTION OF THE INVENTION

The present invention relates to a method for determining mid-regional pro-adrenomedullin (MR-proADM) in a sample, comprising two labelled monoclonal antibodies that bind (preferably distinct) epitopes of MR-proADM, and detecting a signal by the binding of both antibodies to MR-proADM. The present invention relates further to a monoclonal antibody or antibody fragment that binds mid-regional pro-adrenomedullin (MR-proADM). The invention further relates to diagnostic uses and methods, such as an in vitro method for determining MR-proADM in a sample using an antibody or antibody fragment of the invention. Various related aspects and embodiments are encompassed by the invention.

### Assays and Time-Resolved Energy Transfer:

In embodiments, the antibodies of the invention may be conjugated to one or more labels, such as fluorescent labels, preferably two separate fluorescent labels suitable for application in the KRYPTOR™ assay. In embodiments, the KRYPTOR assay is based on the TRACE technology, which is a non-radiative energy transfer from a donor [a cage-like structure with a europium ion in the center (cryptate)] to an acceptor. The proximity of donor (cryptate) and acceptor in a formed immunocomplex and the spectral overlap between donor emission and acceptor absorption spectra on the one hand intensifies the fluorescent signal and on the other hand extends the life span of the acceptor signal, allowing for the measurement of temporally delayed fluorescence.

After the sample to be measured has been excited with a nitrogen laser at a suitable wavelength, the donor (cryptate) emits a long-life fluorescent signal in the millisecond range, while the acceptor generates a short-life signal in the range of nanoseconds. When both components are bound in an immunocomplex, both the signal amplification and the prolonged life span of the acceptor signal occurs, and the life is in the microsecond range. This delayed acceptor signal is proportional to the concentration of the analyte to be measured. The specific fluorescence which is proportional to the antigen concentration is obtained through a double selection: spectral (separation depending on wave-length) and temporal (time resolved measurement). This enables an exclusive measurement of the signal emitted by the immunological complex and the ratio between the two wave-lengths allows a real-time correction of the variations in optic transmission from the medium.

In embodiments, the assay is homogenous and does not require separation or washing steps. It is thus possible to obtain data without interrupting the immunological reaction.

In other embodiments, the antibodies may be employed in a method selected from the group consisting of luminescence immunoassay (LIA), radioimmunoassay (RIA), chemiluminescence- and fluorescence-immunoassays, enzyme immunoassay (EIA), Enzyme-linked immunoassays (ELISA), luminescence-based bead arrays, magnetic beads based arrays, protein microarray assays, rapid test formats such as for instance immunochromatographic strip tests, rare cryptate assay, and automated systems/analyzers.

The antibodies may be employed in a method embodied as a homogeneous method, wherein the sandwich complexes formed by the antibody/antibodies and the marker, e.g., the proADM or a fragment thereof, which is to be detected remains suspended in the liquid phase. In this case it is preferred, that when two antibodies are used, both antibodies are labelled with parts of a detection system, which leads to generation of a signal or triggering of a signal if both antibodies are integrated into a single sandwich.

Such techniques are to be embodied in particular as fluorescence enhancing or fluorescence quenching detection methods. A particularly preferred aspect relates to the use of detection reagents which are to be used pairwise, such as for example the ones which are described in U.S. Pat. No. 4,882,733 A, EP-B1 0 180 492 or EP-B1 0 539 477 and the prior art cited therein. In this way, measurements in which only reaction products comprising both labelling components in a single immune-complex directly in the reaction mixture are detected, become possible.

For example, such technologies are offered under the brand names TRACE™ (Time Resolved Amplified Cryptate Emission) or KRYPTOR™, implementing the teachings of the above-cited applications. Therefore, in particular preferred aspects, a diagnostic device is used to carry out the herein provided method. For example, the level of the proADM protein or a fragment thereof, and/or the level of any further marker of the herein provided method are determined. In particular preferred aspects, the diagnostic device is KRYPTOR.

In one embodiment of the method described herein the method is an immunoassay and wherein the assay is performed in homogeneous phase or in heterogeneous phase. In one embodiment of the method described herein, a first antibody and a second antibody (wherein preferably at least one and more preferably both are antibodies described herein) are present dispersed in a liquid reaction mixture, and wherein a first labelling component which is part of a labelling system based on fluorescence or chemiluminescence extinction or amplification is bound to the first antibody, and a second labelling component of said labelling system is bound to the second antibody so that, after binding of both antibodies to said MR-proADM or fragments thereof to be detected, a measurable signal which permits detection of the resulting sandwich complexes in the measuring solution is generated.

In one embodiment of the method described herein the labelling system comprises a rare earth cryptate or chelate in combination with a fluorescent or chemiluminescent dye, in particular of the cyanine type.

By way of example, two types of cages have been developed to complex lanthanides and enable the labeling of the receptor of interest: (i) chelates display high affinity for europium and terbium ions but the complexation is reversible and can be impacted by the presence of other ions such as Mn2+, Mg2+, or Ca2+; (ii) cryptates, by contrast, offer a greater stability since terbium and europium cannot be released after complexation.

An example of structure of cryptate, Terbium cryptate, is illustrated below:

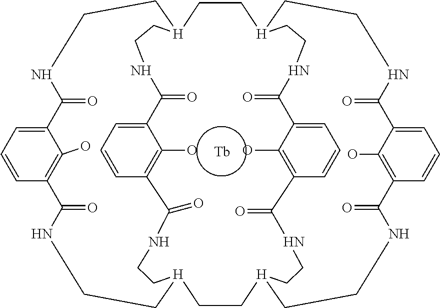

Upon excitation, lanthanide fluorescence half time is in the range of 1 ms while it is in the range of few nanoseconds for classic fluorophores. TR-FRET and TRACE take advantage of this property: the introduction of a time delay (typically around 50 μs) between the excitation and the fluorescence signal detection allows discriminating between short-lived and longer-lasting fluorescence. Therefore, all short-lived fluorescence provided by the medium, the biological preparation or the direct excitation of the acceptor will be eliminated by the time delay. Only the long-lived fluorescence resulting from the donor or the acceptor engaged in a FRET process will be measured after the time delay.

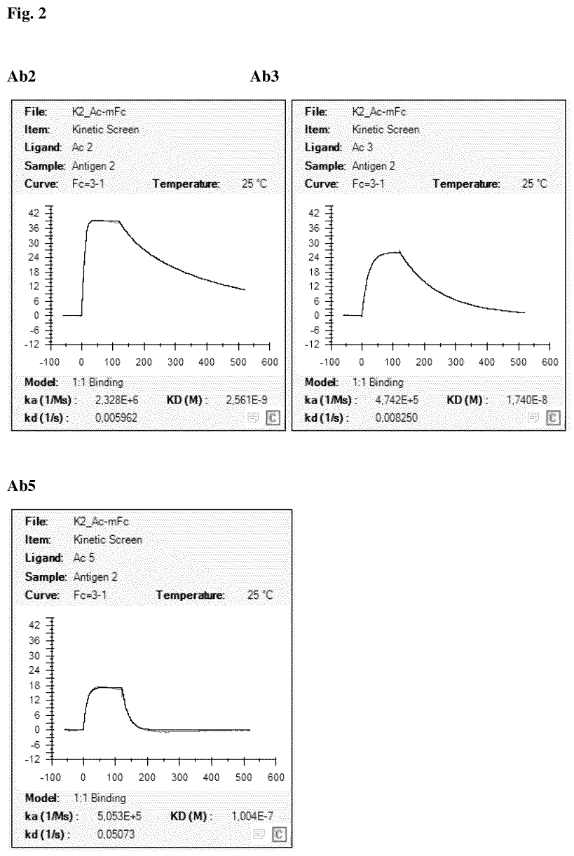

Both europium and terbium cryptates are excited at 300-350 nm. They both exhibit an important Stoke shift and complex emission spectra with multiple fluorescent peaks. For example, europium cryptate trisbipyridine [TBP(Eu)] exhibits four major fluorescent peaks at 585, 605, 620, and 700 nm, while the europium pyridine bisbipyridine (Eu-PBP) has two major peaks at 595 and 615 nm, and two minor peaks at 680 and 705 nm. Terbium cryptate also displays four emission peaks around 490, 550, 585, and 620 nm (FIG. 2C). This makes europium and terbium cryptates compatible with deep red Cy5- or dy647-like fluorophores. Moreover because of the emission peak around 490 nm, terbium-cryptate is also compatible with fluorescein-like fluorophore as acceptor.

Non-Limiting Examples of Acceptor Fluorophores are (Derived from Www.Abcam.Com/Technical):

| | Absorbance | Emission | Visible |
|---|---|---|---|
| Dye | Wavelength | Wavelength | color |
| Cy2 | 490 | 510 | green (dark) |
| FAM | 495 | 516 | green (dark) |
| Alexa fluor 488 | 494 | 517 | green (light) |
| Fluorescein FITC | 495 | 518 | green (light) |
| Alexa fluor 430 | 430 | 545 | green (light) |
| Alexa fluor 532 | 530 | 555 | green (light) |
| HEX | 535 | 556 | green (light) |
| Tamara | 565 | 580 | red |
| Cy3.5 581 | 581 | 596 | red |
| Rox | 575 | 602 | red |
| Alexa fluor 568 | 578 | 603 | red |
| Red 613 | 480; 565 | 613 | red |
| Texas Red | 615 | 615 | red |
| Alexa fluor 594 | 590 | 617 | rec |
| Alexa fluor 633 | 621 | 639 | red |
| Allophycocyanin | 650 | 660 | red |
| Alexa fluor 633 | 650 | 668 | red |
| Cy5 | 650 | 670 | red |
| Alexa fluor 660 | 663 | 690 | red |
| Cy5.5 | 675 | 694 | red |
| TruRed | 490; 675 | 695 | red |
| Alexa fluor 680 | 679 | 702 | red |
| Cy7 | 743 | 770 | red |

### Antibodies:

As used herein, an “antibody” generally refers to a protein consisting of one or more polypeptides substantially encoded by immunoglobulin genes or fragments of immunoglobulin genes. Where the term “antibody” is used, the term “antibody fragment” may also be considered to be referred to. The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. The basic immunoglobulin (antibody) structural unit is known to comprise a tetramer or dimer. Each tetramer is composed of two identical pairs of polypeptide chains, each pair having one “light” (L) (about 25 kD) and one “heavy” (H) chain (about 50-70 kD). The N-terminus of each chain defines a variable region of about 100 to 110 or more amino acids, primarily responsible for antigen recognition. The terms “variable light chain” and “variable heavy chain” refer to these variable regions of the light and heavy chains respectively. Optionally, the antibody or the immunological portion of the antibody, can be chemically conjugated to, or expressed as, a fusion protein with other proteins.

In embodiments, the antibodies are specific for or bind specifically to MR-proADM. “Specific binding” is to be understood as via one skilled in the art, whereby the skilled person is clearly aware of various experimental procedures that can be used to test binding and binding specificity. Some cross-reaction or background binding may be inevitable in many protein-protein interactions; this is not to detract from the “specificity” of the binding between antibody and epitope. The term “directed against” is also applicable when considering the term “specificity” in understanding the interaction between antibody and epitope. In embodiments, an antibody is considered to be specific, if its affinity towards the molecule of interest, e.g. MR-proADM, or the fragment thereof is at least 50-fold higher, preferably 100-fold higher, most preferably at least 1000-fold higher than towards other molecules comprised in a sample containing the molecule of interest. It is well known in the art how to develop and to select antibodies with a given specificity. In the context of the invention, monoclonal antibodies are preferred. The antibody or the antibody binding fragment binds specifically to the herein defined markers or fragments thereof. In particular, the antibody or the antibody binding fragment binds to the herein defined peptides of MR-proADM. Thus, the herein defined peptides can also be epitopes to which the antibodies specifically bind. Further, an antibody or an antibody binding fragment is used in the methods and kits of the invention that binds specifically to MR-proADM.

Antibodies of the invention include, but are not limited to polyclonal, monoclonal, bispecific, human or chimeric antibodies, single variable fragments (ssFv), single domain antibodies (such as VHH fragments from nanobodies), single chain fragments (scFv), Fab fragments, F(ab′)₂ fragments, fragments produced by a Fab expression library, anti-idiotypic antibodies and epitope-binding fragments or combinations thereof of any of the above, provided that they retain the original binding properties. Also mini-antibodies and multivalent antibodies such as diabodies, triabodies, tetravalent antibodies and peptabodies can be used in a method of the invention. The immunoglobulin molecules of the invention can be of any class (i.e. IgG, IgE, IgM, IgD and IgA) or subclass of immunoglobulin molecules. Thus, the term antibody, as used herein, also includes antibodies and antibody fragments either produced by the modification of whole antibodies or synthesized de novo using recombinant DNA methodologies.

The present invention further relates to the use of the antibodies, or fragments thereof, as described herein, for example the variable regions, in recognition molecules or affinity reagents that are suitable for selective binding to a target. The affinity reagent, antibody or fragment thereof according to the invention may be PEGylated, whereby PEGylation refers to covalent attachment of polyethylene glycol (PEG) polymer chains to the inventive antibody. PEGylation may be routinely achieved by incubation of a reactive derivative of PEG with the target molecule. PEGylation to the antibody can potentially mask the agent from the host's immune system, leading to reduced immunogenicity and antigenicity or increase the hydrodynamic size of the agent which may prolong its circulatory time by reducing renal clearance.

A variable region of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Allazikani et al. (1997) J. Molec. Biol. 273:927-948). As used herein, a CDR may refer to CDRs defined by either approach or by a combination of both approaches, or other suitable approach.

In some embodiments, the invention provides an antibody, which comprises at least one CDR, at least two, at least three, or more CDRs that are substantially identical to at least one CDR, at least two, at least three, or more CDRs of the antibody of the invention. Other embodiments include antibodies which have at least two, three, four, five, or six CDR(s) that are substantially identical to at least two, three, four, five or six CDRs of the antibodies of the invention or derived from the antibodies of the invention. In some embodiments, the at least one, two, three, four, five, or six CDR(s) are at least about 80%, 85%, 86%, 87%, 88%, 89%, 90%, 95%, 96%, 97%, 98%, or 99% identical to at least one, two or three CDRs of the antibody of the invention. It is understood that, for purposes of this invention, binding specificity and/or overall activity is generally retained, although the extent of activity may vary compared to said antibody (may be greater or lesser).

The half-life and cytotoxic potential of an antibody are dependent primarily on the interaction of the Fc-domain with different Fc-gamma-receptors. In the case of the antibody half-life, the neonatal Fc receptor (FcRn) plays a major role. This receptor is expressed on several cell types and tissues such as monocytes and vascular endothelia cells that are able to take up serum proteins into their recycling endosomes. In the endosomes, the pH is decreased to approximately 6 and under these conditions the antibodies are able to bind to FcRn. This interaction protects the antibodies from degradation until they are again released into the blood where the physiological pH disrupts the binding to the receptor (Roopenian and Akilesh (2007) Nat Rev Immunol 7:715-725). The higher the affinity of the antibody to the FcRn at pH 6, the greater the half-life of that antibody. Fc-fragment mutations known to stabilize this interaction are summarised in Presta (2008, Curr Opin Immunol 20:460-470).

The invention further relates to “MR-proADM binders” or “affinity reagents” or other “binding agents” potentially based on the structures and/or sequences described herein.

### Sequence Variation:

Sequence variants of the claimed nucleic acids, proteins and antibodies, for example defined by the claimed % sequence identity, that maintain the said properties of the invention are also included in the scope of the invention. Such variants, which show alternative sequences, but maintain essentially the same binding properties, such as target specificity, as the specific sequences provided are known as functional analogues, or as functionally analogous. Sequence identity relates to the percentage of identical nucleotides or amino acids when carrying out a sequence alignment.

It will be appreciated by those of ordinary skill in the art that, as a result of the degeneracy of the genetic code, there are many nucleotide sequences that encode a polypeptide as described herein. Some of these polynucleotides bear minimal homology or sequence identity to the nucleotide sequence of any native gene. Nonetheless, polynucleotides that vary due to differences in codon usage are specifically contemplated by the present invention. Deletions, substitutions and other changes in sequence that fall under the described sequence identity are also encompassed in the invention.

Protein sequence modifications, which may occur through substitutions, are also included within the scope of the invention. Substitutions as defined herein are modifications made to the amino acid sequence of the protein, whereby one or more amino acids are replaced with the same number of (different) amino acids, producing a protein which contains a different amino acid sequence than the primary protein, preferably without significantly altering the function of the protein. Like additions, substitutions may be natural or artificial. It is well known in the art that amino acid substitutions may be made without significantly altering the protein's function. This is particularly true when the modification relates to a “conservative” amino acid substitution, which is the substitution of one amino acid for another of similar properties. Such “conserved” amino acids can be natural or synthetic amino acids which because of size, charge, polarity and conformation can be substituted without significantly affecting the structure and function of the protein. Frequently, many amino acids may be substituted by conservative amino acids without deleteriously affecting the protein's function.

In general, the non-polar amino acids Gly, Ala, Val, Ile and Leu; the non-polar aromatic amino acids Phe, Trp and Tyr; the neutral polar amino acids Ser, Thr, Cys, Gln, Asn and Met; the positively charged amino acids Lys, Arg and His; the negatively charged amino acids Asp and Glu, represent groups of conservative amino acids. This list is not exhaustive. For example, it is well known that Ala, Gly, Ser and sometimes Cys can substitute for each other even though they belong to different groups.

Substitution variants have at least one amino acid residue in the antibody molecule removed and a different residue inserted in its place. The sites of greatest interest for substitutional mutagenesis include the hypervariable regions, but FR alterations are also contemplated. If such substitutions result in a change in biological activity, then more substantial changes, denominated “exemplary substitutions” in the table immediately below, or as further described below in reference to amino acid classes, may be introduced and the products screened.

### Potential Amino Acid Substitutions:

| | Preferred | |
|---|---|---|
| Original | conservative | Examples of exemplary |
| residue | substitutions | conservative substitutions |
| Ala (A) | Val | Val; Leu; Ile |
| Asg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn, Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; Norleucine |
| Leu (L) | Ile | Norleucine; Ile; Val; Met; Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; Phe; Ala; Norleucine |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain.

Conservative amino acid substitutions are not limited to naturally occurring amino acids, but also include synthetic amino acids. Commonly used synthetic amino acids are omega amino acids of various chain lengths and cyclohexyl alanine which are neutral non-polar analogs; citrulline and methionine sulfoxide which are neutral non-polar analogs, phenylglycine which is an aromatic neutral analog; cysteic acid which is a negatively charged analog and ornithine which is a positively charged amino acid analog. Like the naturally occurring amino acids, this list is not exhaustive, but merely exemplary of the substitutions that are well known in the art.

### Antibody Production:

The antibodies of the present invention may be produced by any suitable method, for example by transfection of a host cell with an expression vector comprising the coding sequence for the antibody of the invention. An expression vector or recombinant plasmid is produced by placing these coding sequences for the antibody in operative association with conventional regulatory control sequences capable of controlling the replication and expression in, and/or secretion from, a host cell. Regulatory sequences include promoter sequences, e.g., CMV promoter, and signal sequences which can be derived from other known antibodies. Similarly, a second expression vector can be produced having a DNA sequence which encodes a complementary antibody light or heavy chain. In certain embodiments this second expression vector is identical to the first except insofar as the coding sequences and selectable markers are concerned, so to ensure as far as possible that each polypeptide chain is functionally expressed. Alternatively, the heavy and light chain coding sequences for the antibody may reside on a single vector.

A selected host cell is co-transfected by conventional techniques with both the first and second vectors (or simply transfected by a single vector) to create the transfected host cell of the invention comprising both the recombinant or synthetic light and heavy chains. The transfected cell is then cultured by conventional techniques to produce the engineered antibody of the invention. The antibody which includes the association of both the recombinant heavy chain and/or light chain is screened from culture by appropriate assay, such as ELISA or RIA. Similar conventional techniques may be employed to construct other antibodies.

Suitable vectors for the cloning and subcloning steps employed in the methods and construction of the compositions of this invention may be selected by one of skill in the art. For example, the conventional pUC series of cloning vectors may be used. One vector, pUC19, is commercially available. The components of such vectors, e.g. replicons, selection genes, enhancers, promoters, signal sequences and the like, may be obtained from commercial or natural sources or synthesized by known procedures for use in directing the expression and/or secretion of the product of the recombinant DNA in a selected host. Other appropriate expression vectors of which numerous types are known in the art for mammalian, bacterial, insect, yeast, and fungal expression may also be selected for this purpose.

The present invention also encompasses a cell line transfected with a recombinant plasmid containing the coding sequences of the antibodies of the present invention. Host cells useful for the cloning and other manipulations of these cloning vectors are also conventional.

Suitable host cells or cell lines for the expression of the antibodies of the invention include mammalian cells such as NS0, Sp2/0, CHO (e.g. DG44), COS, HEK, a fibroblast cell (e.g., 3T3), and myeloma cells, for example it may be expressed in a CHO or a myeloma cell. Human cells may be used, thus enabling the molecule to be modified with human glycosylation patterns. Alternatively, other prokaryotic or eukaryotic cell lines may be employed. The selection of suitable mammalian host cells and methods for transformation, culture, amplification, screening and product production and purification are known in the art.

In accordance with the present invention there is provided a method of producing an antibody of the present invention which binds to MR-proADM, which method comprises the steps of; providing a first vector encoding a heavy chain of the antibody; providing a second vector encoding a light chain of the antibody; transforming a mammalian host cell (e.g. CHO) with said first and second vectors; culturing the host cell of step (c) under conditions conducive to the secretion of the antibody from said host cell into said culture media; recovering the secreted antibody of step (d). Once expressed, the antibody can be assessed for the desired binding properties using methods as described herein.

Antibodies and their Use in Immunoassays:

In embodiments, the level of MR-proADM or fragments thereof can be determined by any assay that determines the concentration of the marker by using the inventive antibodies.

Particularly, immunoassays based on the inventive antibodies can be employed as exemplified in the appended examples. As used herein, an immunoassay is a biochemical test that measures the presence or concentration of a macromolecule/polypeptide in a solution through the use of an antibody or antibody binding fragment or immunoglobulin.

Further, an antibody or an antibody binding fragment can in embodiments be used in the methods and kits of the invention that binds specifically to proADM or fragments thereof and optionally to other markers of the present inventions such as PCT. Exemplary immunoassays can be luminescence immunoassay (LIA), radioimmunoassay (RIA), chemiluminescence- and fluorescence-immunoassays, enzyme immunoassay (EIA), Enzyme-linked immunoassays (ELISA), luminescence-based bead arrays, magnetic beads based arrays, protein microarray assays, rapid test formats, rare cryptate assay. Further, assays suitable for point-of-care testing and rapid test formats such as for instance immune-chromatographic strip tests can be employed. Automated immunoassays are also intended, such as the KRYPTOR™ assay.

In certain aspects of the invention, the method is an immunoassay comprising the steps of:
a) contacting the sample with
   i. a first antibody or an antigen-binding fragment or derivative thereof specific for a first epitope of said proADM, and
   ii. a second antibody or an antigen-binding fragment or derivative thereof specific for a second epitope of said proADM; and
b) detecting the binding of the two antibodies or antigen-binding fragments or derivates thereof to said proADM.

In embodiments, the first antibody and the second antibody can be present dispersed in a liquid reaction mixture, and wherein a first labeling component which is part of a labeling system based on fluorescence or chemiluminescence extinction or amplification is bound to the first antibody, and a second labeling component of said labeling system is bound to the second antibody so that, after binding of both antibodies to said proADM or fragments thereof to be detected, a measurable signal which permits detection of the resulting sandwich complexes in the measuring solution is generated. The labeling system can comprise a rare earth cryptate or chelate in combination with a fluorescent or chemiluminescent dye, in particular of the cyanine type.

As used herein, the term “detection reagent”, “binder” or the like include any reagents that are suitable to determine the herein described marker(s). Such exemplary detection reagents are, for example, ligands, e.g. antibodies or fragments thereof, which specifically bind to the peptide or epitopes of the herein described marker(s). Such ligands might be used in immunoassays as described herein. Further reagents that are employed in the immunoassays to determine the level of the marker(s) may also be comprised in the kit and are herein considered as detection reagents. Detection reagents can also relate to reagents that are employed to detect the markers or fragments thereof by MS based methods. Such detection reagent can thus also be reagents, e.g. enzymes, chemicals, buffers, etc, that are used to prepare the sample for the MS analysis. A mass spectrometer can also be considered as a detection reagent. Detection reagents according to the invention can also be calibration solution(s), e.g. which can be employed to determine and compare the level of the marker(s).

In some embodiments, the antibodies may be immobilized on a solid support. Following addition of sample solution, MR-proADM in the patient's sample binds to the solid phase bound antibodies or fragments thereof of the present invention. The MR-proADM which is obtained e.g. from the serum of a patient and bound to the solid phase is subsequently detected using a label, or labelled reagent and optionally quantified, preferably using a further antibody directed against MR-proADM, as disclosed herein. Thus, according to the invention, detection of MR-proADM in this method is achieved using labelled reagents according to the well-known ELISA (Enzyme-Linked Immunosorbent Assay) technology. Labels according to the invention therefore comprise enzymes catalysing a chemical reaction which can be determined by optical means, especially by means of chromogenic substrates, chemiluminescent methods or fluorescent dyes. In another preferred embodiment the autoantibodies are detected by labelling with weakly radioactive substances in radioimmunoassays (RIA) wherein the resulting radioactivity is measured.

The term immunoassay encompasses techniques including, without limitation, enzyme immunoassays (EIA) such as enzyme multiplied immunoassay technique (EMIT), enzyme-linked immunosorbent assay (ELISA), antigen capture ELISA, sandwich ELISA, IgM antibody capture ELISA (MAC ELISA), and microparticle enzyme immunoassay (MEIA); capillary electrophoresis immunoassays (CEIA); radioimmunoassays (RIA); immunoradiometric assays (IRMA); fluorescence polarization immunoassays (FPIA); lateral flow assays (LFA) and chemiluminescence assays (CL).

In another preferred embodiment of the invention, soluble or solid phase-bound antibodies or fragments thereof of the present invention are used to bind MR-proADM. In a second reaction step, further antibodies, such as those of the present invention, directed against MR-proADM are employed, said further antibodies being detectably labelled. The advantage of this embodiment lies in the use of ELISA technology usually available in laboratory facilities so that detection according to the invention can be established in a cost-effective manner. The further antibodies may be detectably coupled to fluorescein isothiocyanate (FITC). Much like the above-mentioned ELISA, the FITC technology represents a system that is available in many places and therefore allows smooth and low-cost establishment of the inventive detection in laboratory routine.

Indirect labels include various enzymes well-known in the art, such as horseradish peroxidase (HRP), alkaline phosphatase (AP), β-galactosidase, urease, and the like. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a β-galactosidase detection system can be used with the chromogenic substrate o-nitrophenyl-β-D-galactopyranoside (ONPG), which yields a soluble product detectable at 410 nm.

In another embodiment of the invention, the antibodies or fragments thereof of the present invention in accordance with one or more of the sequences disclosed herein is bound to a solid phase. Binding of antibodies or fragments thereof of the present invention in accordance with one or more of the sequences disclosed herein to the solid phase can be effected via a spacer. All those chemical compounds having suitable structural and functional preconditions for spacer function can be used as spacers as long as they do not modify the binding behavior in such a way that binding of MR-proADM to antibodies or fragments thereof of the present invention is adversely affected.

In another preferred embodiment of the invention the molecule comprises a linker or spacer selected from the group of α-aminocarboxylic acids as well as homo- and heterooligomers thereof, α,ω-aminocarboxylic acids and branched homo- or heterooligomers thereof, other amino acids, as well as linear and branched homo- or heterooligomers; amino-oligoalkoxyalkylamines; maleinimidocarboxylic acid derivatives; oligomers of alkylamines; 4-alkylphenyl derivatives; 4-oligoalkoxyphenyl or 4-oligoalkoxyphenoxy derivatives; 4-oligoalkylmercaptophenyl or 4-oligoalkylmercaptophenoxy derivatives; 4-oligoalkylaminophenyl or 4-oligoalkylaminophenoxy derivatives; (oligoalkylbenzyl)phenyl or 4-(oligoalkylbenzyl)phenoxy derivatives, as well as 4-(oligoalkoxybenzyl)phenyl or 4-(oligoalkoxybenzyl)phenoxy derivatives; trityl derivatives; benzyloxyaryl or benzyloxyalkyl derivatives; xanthen-3-yloxyalkyl derivatives; (4-alkylphenyl)- or w-(4-alkylphenoxy)alkanoic acid derivatives; oligoalkylphenoxyalkyl or oligoalkoxyphenoxyalkyl derivatives; carbamate derivatives; amines; trialkylsilyl or dialkylalkoxysilyl derivatives; alkyl or aryl derivatives or combinations thereof.

In another embodiment of the invention, the antibodies or fragments thereof of the present invention are immobilized. More specifically, the solid phase-bound antibodies or fragments thereof of the present invention is bound to organic, inorganic, synthetic and/or mixed polymers, preferably agarose, cellulose, silica gel, polyamides and/or polyvinyl alcohols. In the meaning of the invention, immobilization is understood to involve various methods and techniques to fix the antibodies or fragments thereof of the present invention on specific carriers, e.g. according to WO 99/56126 or WO 02/26292. For example, immobilization can serve to stabilize the peptides so that their activity would not be reduced or adversely modified by biological, chemical or physical exposure, especially during storage or in single-batch use. Immobilization of the antibodies or fragments thereof of the present invention allows repeated use under technical or clinical routine conditions; furthermore, a sample—preferably blood components—can be reacted with at least one of the antibodies or fragments thereof of the present invention in a continuous fashion. In the meaning of the invention, three basic methods can be used for immobilization:
(i) Crosslinking: in crosslinking, the antibodies are fixed to one another without adversely affecting their activity. Advantageously, they are no longer soluble as a result of such crosslinking.
(ii) Binding to a carrier: binding to a carrier proceeds via adsorption, ionic binding or covalent binding, for example. Such binding may also take place inside microbial cells or liposomes or other membranous, closed or open structures. Advantageously, the antibodies are not adversely affected by such fixing. For example, multiple or continuous use of carrier-bound antibodies is possible with advantage in clinical diagnosis or therapy.
(iii) Inclusion: inclusion in the meaning of the invention especially proceeds in a semipermeable membrane in the form of gels, fibrils or fibers. Advantageously, encapsulated antibodies are separated from the surrounding sample solution by a semipermeable membrane in such a way that interaction with the virus still is possible. Various methods are available for immobilization, such as adsorption on an inert or electrically charged inorganic or organic carrier.

Another method is covalent binding to carrier materials. In addition, the carriers may have reactive groups forming homopolar bonds with amino acid side chains. Suitable groups in antibodies are carboxy, hydroxy and sulfide groups and especially the terminal amino groups of lysines. Aromatic groups offer the possibility of diazo coupling. Advantageously, a large number of antibodies can undergo direct covalent binding with polyacrylamide resins.

The invention also relates to a diagnostic kit for the determination of MR-proADM, comprising antibodies in accordance with one or more of the sequences as disclosed herein. The diagnostic kit optionally includes instructions concerning combining the contents of the kit and/or providing a formulation for the detection of viral infection. For example, the instruction can be in the form of an instruction leaflet or other medium providing the user with information as to the type of method wherein the substances mentioned are to be used. Obviously, the information need not necessarily be in the form of an instruction leaflet, and the information may also be imparted via the Internet, for example. To a patient, one advantageous effect of such a kit is, for instance, that he or she, without directly addressing a physician, can determine the actual state of a disease.

### Diagnosis, Prognosis, and Therapy Monitoring:

In embodiments, the antibodies or fragments thereof may be employed in a method of diagnosis, prognosis, therapy guidance, risk stratification, or other diagnostic use, to predict the presence of or risk of obtaining, a medical condition associated with MR-proADM.

As used herein, “diagnosis” in the context of the present invention relates to the recognition and (early) detection of a clinical condition. Also, the assessment of the severity may be encompassed by the term “diagnosis”.

“Prognosis” relates to the prediction of an outcome or a specific risk for a subject. This may also include an estimation of the chance of recovery or the chance of an adverse outcome for said subject.

The reagents and methods of the invention may also be used for monitoring, therapy monitoring, therapy guidance and/or therapy control. “Monitoring” relates to keeping track of a patient and potentially occurring complications, e.g. to analyze the progression of the healing process or the influence of a particular treatment or therapy on the health state of the patient.

The term “therapy monitoring” or “therapy control” in the context of the present invention refers to the monitoring and/or adjustment of a therapeutic treatment of said patient, for example by obtaining feedback on the efficacy of the therapy. As used herein, the term “therapy guidance” refers to application of certain therapies, therapeutic actions or medical interventions based on the value/level of one or more biomarkers and/or clinical parameter and/or clinical scores. This includes the adjustment of a therapy or the discontinuation of a therapy.

In the present invention, the terms “risk assessment” and “risk stratification” relate to the grouping of subjects into different risk groups according to their further prognosis. Risk assessment also relates to stratification for applying preventive and/or therapeutic measures. The term “therapy stratification” in particular relates to grouping or classifying patients into different groups, such as risk groups or therapy groups that receive certain differential therapeutic measures depending on their classification. The term “therapy stratification” also relates to grouping or classifying patients with infections or having symptoms of an infectious disease into a group that are not in need to receive certain therapeutic measures.

The sensitivity and specificity of a diagnostic and/or prognostic test depends on more than just the analytical “quality” of the test, they also depend on the definition of what constitutes an abnormal result. In practice, Receiver Operating Characteristic curves (ROC curves), are typically calculated by plotting the value of a variable versus its relative frequency in “normal” (i.e. apparently healthy individuals not having an infection and “disease” populations, e.g. subjects having an infection. For any particular marker (like ADM), a distribution of marker levels for subjects with and without a disease/condition will likely overlap. Under such conditions, a test does not absolutely distinguish normal from disease with 100% accuracy, and the area of overlap might indicate where the test cannot distinguish normal from disease. A threshold is selected, below which the test is considered to be abnormal and above which the test is considered to be normal or below or above which the test indicates a specific condition, e.g. infection. The area under the ROC curve is a measure of the probability that the perceived measurement will allow correct identification of a condition. ROC curves can be used even when test results do not necessarily give an accurate number. As long as one can rank results, one can create a ROC curve. For example, results of a test on “disease” samples might be ranked according to degree (e.g. 1=low, 2=normal, and 3=high). This ranking can be correlated to results in the “normal” population, and a ROC curve created. These methods are well known in the art; see, e.g., Hanley et al. 1982. Radiology 143: 29-36. Preferably, a threshold is selected to provide a ROC curve area of greater than about 0.5, more preferably greater than about 0.7, still more preferably greater than about 0.8, even more preferably greater than about 0.85, and most preferably greater than about 0.9. The term “about” in this context refers to +/−5% of a given measurement.

The horizontal axis of the ROC curve represents (1-specificity), which increases with the rate of false positives. The vertical axis of the curve represents sensitivity, which increases with the rate of true positives. Thus, for a particular cut-off selected, the value of (1-specificity) may be determined, and a corresponding sensitivity may be obtained. The area under the ROC curve is a measure of the probability that the measured marker level will allow correct identification of a disease or condition. Thus, the area under the ROC curve can be used to determine the effectiveness of the test.

### Sample Types:

As used herein, the term “sample” is a biological sample that is obtained or isolated from the patient or subject. “Sample” as used herein may, e.g., refer to a sample of bodily fluid or tissue obtained for the purpose of analysis, diagnosis, prognosis, or evaluation of a subject of interest, such as a patient. Preferably herein, the sample is a sample of a bodily fluid, such as blood, serum, plasma, cerebrospinal fluid, urine, saliva, sputum, pleural effusions, cells, a cellular extract, a tissue sample, any tissue sample from the upper or lower respiratory tract, a tissue biopsy, a stool sample and the like. Particularly, the sample is blood, blood plasma, blood serum.

“Plasma” in the context of the present invention is the virtually cell-free supernatant of blood containing anticoagulant obtained after centrifugation. Exemplary anticoagulants include calcium ion binding compounds such as EDTA or citrate and thrombin inhibitors such as heparinates or hirudin. Cell-free plasma can be obtained by centrifugation of the anticoagulated blood (e.g. citrated, EDTA or heparinized blood), for example for at least 15 minutes at 2000 to 3000 g.

“Serum” in the context of the present invention is the liquid fraction of whole blood that is collected after the blood is allowed to clot. When coagulated blood (clotted blood) is centrifuged serum can be obtained as supernatant.

As used herein, “urine” is a liquid product of the body secreted by the kidneys through a process called urination (or micturition) and excreted through the urethra.

### Adrenomedullin:

It is understood that in the context of the present invention “determining a level of proADM or fragment(s) thereof” or the like refers to any means of determining preferably MR-proADM or a fragment thereof. The fragment can have any length, e.g. at least about 5, 10, 20, 30, 40, 50 or 100 amino acids, so long as the fragment allows the unambiguous determination of the level of MR-proADM. In particular preferred aspects of the invention, “determining the level of proADM” refers to determining the level of midregional proadrenomedullin (MR-proADM). MR-proADM is a fragment and/or region of proADM.

The peptide adrenomedullin (ADM) was discovered as a hypotensive peptide comprising 52 amino acids, which had been isolated from a human phenochromocytome (Kitamura et al., 1993). Adrenomedullin (ADM) is encoded as a precursor peptide comprising 185 amino acids (“preproadrenomedullin” or “pre proADM”). An exemplary amino acid sequence of ADM is given in SEQ ID NO: 1.

ADM comprises the positions 95-146 of the pre-proADM amino acid sequence and is a splice product thereof. “Proadrenomedullin” (“proADM” SEQ ID NO 2) refers to pre-proADM without the signal sequence (amino acids 1 to 21), i.e. to amino acid residues 22 to 185 of pre-proADM. “Midregional proadrenomedullin” (“MR-proADM”) refers to the amino acids 42 to 95 of pre-proADM. An exemplary amino acid sequence of MR-proADM is given in SEQ ID NO: 3.

It is also envisaged herein that a peptide and fragment thereof of pre-proADM or MR-proADM can be used for the herein described methods. For example, the peptide or the fragment thereof can comprise the amino acids 22-41 of pre-proADM (PAMP peptide) or amino acids 95-146 of pre-proADM (mature adrenomedullin, including the biologically active form, also known as bio-ADM, SEQ ID NO 4).

A C-terminal fragment of proADM (amino acids 153 to 185 of pre proADM) is called adrenotensin. Fragments of the proADM peptides or fragments of the MR-proADM can comprise, for example, at least about 5, 10, 20, 30 or more amino acids. Accordingly, the fragment of proADM may, for example, be selected from the group consisting of MR-proADM, PAMP, adrenotensin and mature adrenomedullin, preferably herein the fragment is MR-proADM.

The determination of these various forms of MR-proADM or fragments thereof also encompasses measuring and/or detecting specific sub-regions of these molecules, for example by employing antibodies or other affinity reagents directed against a particular portion of the molecules.

### Other Biomarkers or Parameters:

Accordingly, the methods and kits of the present invention can also comprise determining at least one further biomarker, marker, clinical score and/or parameter in addition to ADM.

As used herein, a parameter is a characteristic, feature, or measurable factor that can help in defining a particular system. A parameter is an important element for health- and physiology-related assessments, such as a disease/disorder/clinical condition risk, preferably organ dysfunction(s). Furthermore, a parameter is defined as a characteristic that is objectively measured and evaluated as an indicator of normal biological processes, pathogenic processes, or pharmacologic responses to a therapeutic intervention. An exemplary parameter can be selected from the group consisting of Pneumonia Severity Index (PSI), Acute Physiology and Chronic Health Evaluation II (APACHE II), the simplified acute physiology score (SAPSII score), sequential organ failure assessment score (SOFA score), quick sequential organ failure assessment score (qSOFA), body mass index, weight, age, sex, IGS II, liquid intake, white blood cell count, sodium, potassium, temperature, blood pressure, dopamine, bilirubin, respiratory rate, partial pressure of oxygen, World Federation of Neurosurgical Societies (WFNS) grading, Glasgow Coma Scale (GCS), CURB-65 pneumonia severity score, Pneumonia Severity Index (PSI), age, gender, family history, ethnicity, body weight, body mass index (BMI), cystoscopy report, white blood cell count, lymphocyte count, imaging methods as such as CT scan, PET imaging or X-ray, blood pressure, heart rate, antihypertensive treatment, liquid intake, wheezing, body temperature, presence of diabetes mellitus, blood glucose levels, and (current) smoking habits.

Such parameters may additionally be assessed in combination with the methods described herein in order to improve assay implementation and diagnostic statements.

The present invention has the following advantages over the conventional methods: the inventive methods and the kits are fast, objective, easy to use and precise. The methods and kits of the invention relate to markers and clinical scores that are easily measurable in routine methods, because the levels of proADM, PCT, D-Dimer, Troponin, Copeptin, lactate, c-reactive protein, can be determined in routinely obtained blood samples or further biological fluids or samples obtained from a subject.

As used herein, terms such as “marker”, “surrogate”, “prognostic marker”, “factor” or “biomarker” or “biological marker” are used interchangeably and relate to measurable and quantifiable biological markers (e.g., specific protein or enzyme concentration or a fragment thereof, specific hormone concentration or a fragment thereof, or presence of biological substances or a fragment thereof) which serve as indices for health- and physiology-related assessments, such as a disease/disorder/clinical condition risk, preferably an adverse event. A marker or biomarker is defined as a characteristic that can be objectively measured and evaluated as an indicator of normal biological processes, pathogenic processes, or pharmacologic responses to a therapeutic intervention. Biomarkers may be measured in a sample (as a blood, plasma, urine, or tissue test).

The at least one further marker and/or parameter of said subject can be selected from the group consisting of a level of lactate in said sample, a level of procalcitonin (PCT) in said sample, the sequential organ failure assessment score (SOFA score) of said subject, optionally the quick SOFA score, the simplified acute physiology score (SAPSII) of said subject, the Acute Physiology and Chronic Health Evaluation II (APACHE II) score of said subject and a level of the soluble fms-like tyrosine kinase-1 (sFlt-1), Histone H2A, Histone H2B, Histone H3, Histone H4, calcitonin, Endothelin-1 (ET-1), Arginine Vasopressin (AVP), Atrial Natriuretic Peptide (ANP), Neutrophil Gelatinase-Associated Lipocalin (NGAL), Troponin, Brain Natriuretic Peptide (BNP), C-Reactive Protein (CRP), Pancreatic Stone Protein (PSP), Triggering Receptor Expressed on Myeloid Cells 1 (TREM1), Interleukin-6 (IL-6), Interleukin-1, Interleukin-24 (IL-24), Interleukin-22 (IL-22), Interleukin (IL-20) other ILs, Presepsin (sCD14-ST), Lipopolysaccharide Binding Protein (LBP), Alpha-1-Antitrypsin, Matrix Metalloproteinase 2 (MMP2), Metalloproteinase 2 (MMP8), Matrix Metalloproteinase 9 (MMP9), Matrix Metalloproteinase 7 (MMP7, Placental growth factor (PIGF), Chromogranin A, S100A protein, S100B protein and Tumor Necrosis Factor α (TNFα), Neopterin, Alpha-1-Antitrypsin, pro-arginine vasopressin (AVP, proAVP or Copeptin), procalcitonin, atrial natriuretic peptide (ANP, pro-ANP), Endothelin-1, CCL1/TCA3, CCL11, CCL12/MCP-5, CCL13/MCP-4, CCL14, CCL15, CCL16, CCL17/TARC, CCL18, CCL19, CCL2/MCP-1, CCL20, CCL21, CCL22/MDC, CCL23, CCL24, CCL25, CCL26, CCL27, CCL28, CCL3, CCL3L3, CCL4, CCL4L1/LAG-1, CCL5, CCL6, CCL7, CCL8, CCL9, CX3CL1, CXCL1, CXCL10, CXCL11, CXCL12, CXCL13, CXCL14, CXCL15, CXCL16, CXCL17, CXCL2/MIP-2, CXCL3, CXCL4, CXCL5, CXCL6, CXCL7/Ppbp, CXCL9, IL8/CXCL8, XCL1, XCL2, FAM19A1, FAM19A2, FAM19A3, FAM19A4, FAM19A5, CLCF1, CNTF, IL11, IL31, IL6, Leptin, LIF, OSM, IFNA1, IFNA10, IFNA13, IFNA14, IFNA2, IFNA4, IFNA7, IFNB1, IFNE, IFNG, IFNZ, IFNA8, IFNA5/IFNaG, IFNω/IFNW1, BAFF, 4-1BBL, TNFSF8, CD40LG, CD70, CD95L/CD178, EDA-A1, TNFSF14, LTA/TNFB, LTB, TNFa, TNFSF10, TNFSF11, TNFSF12, TNFSF13, TNFSF15, TNFSF4, TRAIL, IP-10, IL18, IL18BP, IL1A, IL1B, IL1F10, IL1F3/IL1RA, IL1F5, IL1F6, IL1F7, IL1F8, IL1RL2, IL1F9, IL33 or a fragment thereof. Further markers comprise membrane microparticle, platelet count, mean platelet volume (MPV), sCD14-ST, prothrombinase, antithrombin and/antithrombin activity, cationic protein 18 (CAP18), von Willebrand factor (vWF)-cleaving proteases, lipoproteins in combination with CRP, fibrinogen, fibrin, B2GP1, GPIIb-IIIa, non-denatured D-dimer of fibrin, platelet factor 4, histones and a PT-Assay.

In embodiments, the biomarker for combined analysis with MR-proADM using the antibodies described herein is PCT, TRAIL and/or IP-10, preferably in combination with MR-proADM, more preferably by measurement in the sample sample, or on the same analytical system.

As used herein, “procalcitonin” or “PCT” relates to a peptide spanning amino acid residues 1-116, 2-116, 3-116, or fragments thereof, of the procalcitonin peptide. PCT is a peptide precursor of the hormone calcitonin. Thus the length of procalcitonin fragments is at least 12 amino acids, preferably more than 50 amino acids, more preferably more than 110 amino acids. PCT may comprise post-translational modifications such as glycosylation, liposidation or derivatization. Procalcitonin is a precursor of calcitonin and katacalcin. Thus, under normal conditions the PCT levels in the circulation are very low (<about 0.05 ng/ml).

The level of PCT in the sample of the subject can be determined by immunoassays as described herein. As used herein, the level of ribonucleic acid or deoxyribonucleic acids encoding “procalcitonin” or “PCT” can also be determined. Methods for the determination of PCT are known to a skilled person, for example by using products obtained from Thermo Fisher Scientific/B⋅R⋅A⋅H⋅M⋅S GmbH.

Other markers may also be used in combination with determining MR-proADM with the inventive antibodies.

### Pharmaceutical Compositions:

In some embodiments, the invention relates to a pharmaceutical composition comprising the antibody or fragment thereof of the invention together with a pharmaceutically acceptable carrier. A pharmaceutically acceptable carrier in the sense of the present invention may be any non-toxic material that does not significantly interfere in a detrimental sense with the effectiveness of the biological activity of the antibodies of the present invention.

Evidently, the characteristics of the carrier will depend on the route of administration. Such a composition may contain, in addition to the active substance and carrier, diluents, fillers, salts, buffers, stabilizers, solubilizers, and other materials well known in the art. Formulation of pharmaceutically-acceptable excipients and carrier solutions is well-known to those of skill in the art, as is the development of suitable dosing and treatment regimens for using the particular compositions described herein in a variety of treatment regimens, including, without limitation, oral, parenteral, intravenous, sub-cutaneous, intranasal, inhalation, and intra-muscular administration.

The medicament, otherwise known as a pharmaceutical composition, containing the active ingredient (antibody or antibody fragment) may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets.

These excipients may be for example, inert diluents, such as calcium carbonate, sodium carbonate, lactose, calcium phosphate or sodium phosphate; granulating and disintegrating agents, for example corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated. The present invention also refers to a pharmaceutical composition for topical application, oral ingestion, inhalation, or cutaneous, subcutaneous, or intravenous injection. A skilled person is aware of the carriers and additives required for particular application forms.

The medicament, otherwise known as a pharmaceutical composition, containing the active ingredient (antibody or antibody fragment) may be in a form suitable for injection. Modes of administration involving injection comprise, without limitation, Subcutaneous (under the skin), Intramuscular (in a muscle), Intravenous (in a vein) or Intrathecal (around the spinal cord). Antibody therapies are typically administered using intravenous administration, which is a preferred mode of administration of the present invention.

Excipients, as an example of pharmaceutically acceptable carrier, for liquid formulations intended for injection are known in the art and can be selected appropriately by a skilled person. Excipients have been used to increase the stability of a wide range of protein and peptide-based formulations by reducing protein dynamics and motion, increasing the conformational stability of mAbs especially at high concentrations and inhibiting interface-dependent aggregation. Excipients usually inhibit aggregation and protects the protein by adsorbing to the air-liquid interface; for example, the use of surfactants (e.g., polysorbate 20 and 80), carbohydrates (e.g., cyclodextrin derivatives) and amino acids (e.g., arginine and histidine) can help prevent aggregation by this mechanism. Cyclodextrin has been reported to stabilize commercially available antibody-based drugs in a hydrogel formulation. Some of the generally recognized as safe (GRAS) excipients include pluronic F68, trehalose, glycine and amino acids such as arginine, glycine, glutamate and histidine, which are found in a number of commercial protein therapeutic products. By way of example, bevacizumab, 25 mg/mL, contains trehalose dehydrate, sodium phosphate and polysorbate 20. Excipients in subcutaneous trastuzumab, 600 mg, are rHuPH20, histidine hydrochloride, histidine, trehalose dehydrate, polysorbate 20, methionine and water for injection.

When a therapeutically effective amount of the active substance (antibody or antibody fragment) of the invention is administered by intravenous, cutaneous or subcutaneous injection, the active substance may be in the form of a solution, preferably a pyrogen-free, parenterally acceptable aqueous solution.

The preparation of such parenterally acceptable solutions, having due regard to pH, isotonicity, stability, and the like, is within the skill in the art. A preferred pharmaceutical composition for intravenous, cutaneous, or subcutaneous injection should contain, in addition to the active substance, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additives known to those of skill in the art.

The invention also relates to administration of a therapeutically relevant amount of antibody as described herein in the treatment of a subject who has the medical disorders as disclosed herein. As used herein, the term “therapeutically effective amount” means the total amount of each active component of the pharmaceutical composition or method that is sufficient to show a meaningful patient benefit. The amount of active substance in the pharmaceutical composition of the present invention will depend upon the nature and severity of the condition being treated, and on the nature of prior treatments which the patient has undergone. Larger doses may be administered until the optimal therapeutic effect is obtained for the patient, and at that point the dosage is not increased further.

The dose of the antibody administered evidently depends on numerous factors well-known in the art such as, e.g., the chemical nature and pharmaceutical formulation of the antibody, and of body weight, body surface, age and sex of the patient, as well as the time and route of administration. For an adult, the dose may exemplarily be between 0.001 μg and 1 g per day, preferably between 0.1 μg and 100 mg per day, more preferably between 1 μg and 100 mg per day, even more preferably between 5 μg and 10 mg per day. In a continuous infusion, the dose may exemplarily be between 0.01 μg and 100 mg, preferably between 1 μg and 10 mg per kilogram body mass per minute.

### Medical Indications:

In one aspect of the present invention there is provided an antibody or antibody fragment according to the invention as herein described for use in the treatment of a medical condition associated with MR-proADM, for example a medical indication in which MR-proADM levels are over-expressed, or are present in blood samples at elevated levels in comparison to a healthy human population, for example above an average value of a healthy human population, or other suitable cut-off value.

As used herein, the “patient” or “subject” may be a vertebrate. In the context of the present invention, the term “subject” includes both humans and animals, particularly mammals, and other organisms.

In the present invention “treatment” or “therapy” generally means to obtain a desired pharmacological effect and/or physiological effect. The effect may be prophylactic in view of completely or partially preventing a disease and/or a symptom, for example by reducing the risk of a subject having a disease or symptom or may be therapeutic in view of partially or completely curing a disease and/or adverse effect of the disease.

In the present invention, “therapy” includes arbitrary treatments of diseases or conditions in mammals, in particular, humans, for example, the following treatments (a) to (c): (a) Prevention of onset of a disease, condition or symptom in a patient; (b) Inhibition of a symptom of a condition, that is, prevention of progression of the symptom; (c) Amelioration of a symptom of a condition, that is, induction of regression of the disease or symptom.

### Diagnostics, Sepsis and Infections:

In embodiments of the present invention, the method may employ diagnosis, prognosis, therapy management, risk stratification and/or treatment of sepsis, severe sepsis and/or septic shock.

In embodiments, the method may employ diagnosis, prognosis, therapy management, and/or risk stratification for the severity assessment of patients in a clinical setting (general practitioners, ED, ICU, hospital ward), including, without limitation, rule-in/out survival (mortality), complications, companion diagnostics to monitor a treatment (responder/non-responder etc.), and/or to monitor or predict disease progression.

“Sepsis” in the context of the invention refers to a systemic response to infection. Alternatively, sepsis may be seen as the combination of SIRS with a confirmed infectious process or an infection. Sepsis may be characterized as clinical syndrome defined by the presence of both infection and a systemic inflammatory response (Levy M M et al. 2001 SCCM/ESICM/ACCP/ATS/SIS International Sepsis Definitions Conference. Crit Care Med. 2003 April; 31(4):1250-6). The term “sepsis” used herein includes, but is not limited to, sepsis, severe sepsis, and septic shock.

The term “sepsis” used herein includes, but is not limited to, sepsis, severe sepsis, and septic shock. Severe sepsis in refers to sepsis associated with organ dysfunction, hypoperfusion abnormality, or sepsis-induced hypotension. Hypoperfusion abnormalities include lactic acidosis, oliguria and acute alteration of mental status. Sepsis-induced hypotension is defined by the presence of a systolic blood pressure of less than about 90 mm Hg or its reduction by about 40 mm Hg or more from baseline in the absence of other causes for hypotension (e.g. cardiogenic shock). Septic shock is defined as severe sepsis with sepsis-induced hypotension persisting despite adequate fluid resuscitation, along with the presence of hypoperfusion abnormalities or organ dysfunction (Bone et al., CHEST 101(6): 1644-55, 1992).

The term sepsis may alternatively be defined as life-threatening organ dysfunction caused by a dysregulated host response to infection. For clinical operationalization, organ dysfunction can preferably be represented by an increase in the Sequential Organ Failure Assessment (SOFA) score of 2 points or more, which is associated with an in-hospital mortality greater than 10%. Septic shock may be defined as a subset of sepsis in which particularly profound circulatory, cellular, and metabolic abnormalities are associated with a greater risk of mortality than with sepsis alone. Patients with septic shock can be clinically identified by a vasopressor requirement to maintain a mean arterial pressure of 65 mm Hg or greater and serum lactate level greater than 2 mmol/L (>18 mg/dL) in the absence of hypovolemia.

The term “sepsis” used herein relates to all possible stages in the development of sepsis. The term “sepsis” also includes severe sepsis or septic shock based on the SEPSIS-2 definition (Bone et al., 2009). The term “sepsis” also includes subjects falling within the SEPSIS-3 definition (Singer et al., 2016). The term “sepsis” used herein relates to all possible stages in the development of sepsis.

In embodiments of the present invention, the method may employ diagnosis, prognosis, therapy management or risk stratification of an infection or an infectious disease.

As used herein, “infection” within the scope of the invention means a pathological process caused by the invasion of normally sterile tissue or fluid by pathogenic or potentially pathogenic agents/pathogens, organisms and/or microorganisms, and relates preferably to infection(s) by bacteria, viruses, fungi, and/or parasites.

Furthermore, the infection-related complication can be a “nosocomial” infection. Nosocomial infections are also called hospital-acquired infections (HAI) are infections that are acquired in a hospital or other health care facility. To emphasize both hospital and nonhospital settings, it is sometimes instead called a health care-associated infection (HAI or HCAI). Such an infection can be acquired in hospital, nursing home, rehabilitation facility, outpatient clinic, or other clinical settings. Nosocomial infection may be spread to the susceptible patient in the clinical setting by various means. Health care staff can spread infection, in addition to contaminated equipment, bed linens, or air droplets. The infection can originate from the outside environment, another infected patient, staff that may be infected, or in some cases, the source of the infection cannot be determined. In some cases the microorganism originates from the patient's own skin microbiota, becoming opportunistic after surgery or other procedures that compromise the protective skin barrier. Though the patient may have contracted the infection from their own skin, the infection is still considered nosocomial since it develops in the health care setting.

Further, the subject suffering from an infection can suffer from more than one source(s) of infection simultaneously. For example, the subject suffering from an infection can suffer from a bacterial infection and viral infection; from a viral infection and fungal infection; from a bacterial and fungal infection, and from a bacterial infection, fungal infection and viral infection, or suffer from a mixed infection comprising one or more of the infections listed herein, including potentially a superinfection, for example one or more bacterial infections in addition to one or more viral infections and/or one or more fungal infections.

### Medical Treatments:

In the context of the present invention, the term “medical treatment” or “treatment” may represent treatment with the inventive antibody, or a subsequent treatment after use of the inventive antibody in a diagnostic or any other analytic assay.

The term “treatment” comprises various treatments and therapeutic strategies, which comprise, without limitation, anti-inflammatory strategies, administration of ADM-antagonists such as therapeutic antibodies, si-RNA or DNA, the extracorporeal blood purification or the removal of harmful substances via apheresis, dialyses, adsorbers to prevent the cytokine storm, removal of inflammatory mediators, plasma apheresis, administration of vitamins such as vitamin C, surgery, emergency surgery, ventilation like mechanical ventilation and non-mechanical ventilation, to provide the body with sufficient oxygen, for example, focus cleaning procedures, transfusion of blood products, infusion of colloids, organ replacement, such as renal or liver replacement, antibiotic treatment, invasive mechanical ventilation, non-invasive mechanical ventilation, vasopressor use, fluid therapy, apheresis and measures for organ protection. A skilled person is capable of determining which of the treatments described herein require administration in a hospital setting.

A skilled person is also capable of determining which medical conditions, and which degrees of severity of such medical conditions, require treatments only (or primarily) available in hospital settings, for example in the ED or ICU.

Further treatments of the present invention comprise the administration of cells or cell products like stem cells, blood or plasma, and the stabilization of the patients circulation and the protection of endothelial glycocalyx, for example via optimal fluid management strategies, for example to reach normovolemia and prevent or treat hypervolemia or hypovolemia. Moreover, vasopressors or e.g. catecholamine as well as albumin or heparanase inhibition via unfractionated heparin or N-desulfated re-N-acetylated heparin are useful treatments to support the circulation and endothelial layer.

Additionally, medical treatments of the present invention comprise, without limitation, stabilization of the blood clotting, anti-fibrinolytic treatment, iNOS inhibitors, anti-inflammatory agents like hydrocortisone, sedatives and analgetics as well as insuline.

Artificial and mechanical ventilation are effective approaches to enhance proper gas exchange and ventilation and aim to save life during severe hypoxemia. Artificial ventilation relates to assisting or stimulating respiration of the subject. Artificial ventilation may be selected from the group consisting of mechanical ventilation, manual ventilation, extracorporeal membrane oxygenation (ECMO) and noninvasive ventilation (NIV). Mechanical ventilation relates to a method to mechanically assist or replace spontaneous breathing. This may involve a machine called a ventilator. Mechanical ventilation may be High-Frequency Oscillatory Ventilation or Partial Liquid Ventilation.

In a preferred embodiment, the term “medical treatment” or “treatment” comprises antibiotic treatment such as intravenous antibiotic, oral antibiotics or topical antibiotics. In a more preferred embodiment, the term “medical treatment” or “treatment” comprises intravenously applied antibiotic treatment.

Medical treatment also comprises methods to avoid hypothermia which includes the use of warmed intravenous fluids and warm air blankets.

Medical treatment also comprises wound management including haemorrhage control, bleeding control techniques with or without tourniquets, wound cleaning, local anaesthetic application, wound closure techniques where skin adhesive strips, tissue adhesive glue, sutures, staples and wound dressings can be used.

A medical treatment of the present invention may be an antibiotic treatment, wherein one or more “antibiotics” or “antibiotic agents” may be administered if an infection has been diagnosed or prognosed by the method of the invention. Antibiotics or antibiotic agents according to the present invention also encompass potentially the anti-fungal or anti-viral compounds used to treat a diagnosed infection or sepsis.

### General Terms:

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of”.

Thus, the terms “comprising”/“including”/“having” mean that any further component (or likewise features, integers, steps and the like) can/may be present. The term “consisting of” means that no further component (or likewise features, integers, steps and the like) is present.

The term “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

Thus, the term “consisting essentially of” means those specific further components (or likewise features, integers, steps and the like) can be present, namely those not materially affecting the essential characteristics of the composition, device or method. In other words, the term “consisting essentially of” (which can be interchangeably used herein with the term “comprising substantially”), allows the presence of other components in the composition, device or method in addition to the mandatory components (or likewise features, integers, steps and the like), provided that the essential characteristics of the device or method are not materially affected by the presence of other components.

The term “method” refers to manners, means, techniques and procedures for accomplishing a given task including, but not limited to, those manners, means, techniques and procedures either known to, or readily developed from known manners, means, techniques and procedures by practitioners of the chemical, biological and biophysical arts.

SEQUENCES
Preferred sequences of the invention:

| SEQ | | |
|---|---|---|
| ID NO: | Sequence (N-C) | Comments |
| 1 | MKLVSVALMYLGSLAFLGADTARLDVASEFRKKWNK | Amino acid sequence of pre- |
| | WALSRGKRELRMSSSYPTGLADVKAGPAQTLIRPQD | pro-ADM, 185 AS |
| | MKGASRSPEDSSPDAARIRVKRYRQSMNNFQGLRSF | |
| | GCRFGTCTVQKLAHQIYQFTDKDKDNVAPRSKISPQ | |
| | GYGRRRRRSLPEAGPGRTLVSSKPQAHGAPAPPSG | |
| | SAPHFL | |
| 2 | ARLDVASEFRKKWNKWALSRGKRELRMSSSYPTGL | Amino acid sequence of pro- |
| | ADVKAGPAQTLIRPQDMKGASRSPEDSSPDAARIRV | ADM (AS 22-185 of pre-pro- |
| | KRYRQSMNNFQGLRSFGCRFGTCTVQKLAHQIYQFT | ADM) |
| | DKDKDNVAPRSKISPQGYGRRRRRSLPEAGPGRTLV | |
| | SSKPQAHGAPAPPSGSAPHFL | |
| 3 | ELRMSSSYPTGLADVKAGPAQTLIRPQDMKGASRSP | Amino acid sequence of MR- |
| | EDSSPDAARIRV | pro-ADM (AS 45-92 of pre-pro- |
| | | ADM) |
| 4 | YRQSMNNFQGLRSFGCRFGTCTVQKLAHQIYQFTDK | Amino acid sequence of |
| | DKDNVAPRSKISPQGY | mature ADM (AS 95-146 of |
| | | pre-pro-ADM) |
| 5 | LADVKAGPAQTLIR | Epitope Ab1 |
| 6 | CLADVKAGPAQTLIRC | Immunizing peptide Ab1 |
| 7 | RPQDMKGASRSPED | Epitope Ab2 |
| 8 | CRPQDMKGASRSPED | Immunizing peptide Ab2 |
| 9 | SSPDAARIRVKR | Epitope Ab3 |
| 10 | KLHCSSPDAARIRVKR | Immunizing peptide Ab3 |
| 11 | PEDSSPDAARIR | Epitope Ab4 |
| 12 | KLHCPEDSSPDAARIR | Immunizing peptide Ab4 |
| 13 | SSPDAARIRVKR | Epitope Ab5 |
| 14 | KLHCSSPDAARIRVKR | Immunizing peptide Ab5 |
| 15 | LADVKAGPAQTLIR | Epitope Ab6 |
| 16 | LADVKAGPAQTLIRCKLH | Immunizing peptide Ab6 |
| 17 | LADVKAGPAQTLIR | Epitope Ab7 |
| 18 | LADVKAGPAQTLIRCKLH | Immunizing peptide Ab7 |
| 19 | EVQLQQSGPELVKPGASVKMSTTTSGYTFTEYTLHW | Alternative Ab1 heavy |
| | VKPSALFTSSLEWLGSLLPKNNSSPPRSGGKALLTVN | |
| | KSSSTAYMELRSLTSDDSAVYYPMGAATTGDQGTSH | |
| | HMRSPMHWFPNWGQGTLVTVSAAKTTPPSVYPLAP | |
| | GPFGCSPDWAFNLKGYFPESVTVTWNSGSLSSSVHT | |
| | FPALLQSGLYPESSPKSVGGMSDGFGASGSEEDATG | |
| | DATQGSTRDKKLEPSGPISTINPCAASHECKMMAPNL | |
| | EGGPSVFIFPPNIKDVLMISLTPKVWNKSMLEPMGDP | |
| | QVDIEDALAGQKAVFNDYFSDFSGVYSAGLSRVVSTL | |
| | PIQHQDWMSGKEFKCKVNNKDLPSPIERTISKIKGLV | |
| | RAPQVYILPPPAEQLSRKRAAYDRHAYKAVLSEGQLQ | |
| | QSGPELVKPAGSVKDTAPVLDSDGSYFIYSKLNMKPH | |
| | GGWEKTDSFSPHHTGLKNYYLKKTISRSPGK | |
| 20 | DVPYTQTPLSLPVSLGDQASISFNSSQSIVHSNGNTY | Alternative Ab1 light |
| | LEWYLQKPGQSPKLLIYKVSNRFSGVPDRFSGSGSG | |
| | TDFTLKISRVEAEDLGVYYTDQGSHVPWTFGGGTKL | |
| | EIKRADAAPTVSIFPPSSEKYSQGNVSAVGVTYDGHT | |
| | ALTRDIDNVKWKIDGSERQNGVLNSWTDQDSKDSTY | |
| | SMSSTLTLTKDEYERHNSYTPDGPPATSTSPIVKSTN | |
| | EC | |
| 21 | EVQLQQSGPELVKPGASVKMSTTTSGYTFTEYTLHW | Alternative Ab1 VH |
| | VKPSALFTSSLEWLGSLLPKNNSSPPRSGGKALLTVN | |
| | KSSSTAYMELRSLTSDDSAVYYPMGAATTGDQGTSH | |
| | HMRSPMHWFPNWGQGTLVTVSA | |
| 22 | DVPYTQTPLSLPVSLGDQASISFNSSQSIVHSNGNTY | Alternative Ab1 VL |
| | LEWYLQKPGQSPKLLIYKVSNRFSGVPDRFSGSGSG | |
| | TDFTLKISRVEAEDLGVYYTDQGSHVPWTFGGGTKL | |
| | EIK | |
| 23 | GYTFTEYTLH | Alternative Ab1 H-CDR1-a |
| 24 | SGYTFTEYTLHW | Alternative Ab1 H-CDR1-b |
| 25 | GYTFTEYT | Alternative Ab1 H-CDR1-c |
| 26 | SLLPKNNSSPPRSGG | Alternative Ab1 H-CDR2-a |
| 27 | GSLLPKNNSSPPRSGGK | Alternative Ab1 H-CDR2-b |
| 28 | LLPKNN | Alternative Ab1 H-CDR2-c |
| 29 | TGDQGTSHHMRSPMHWFPN | Alternative Ab1 H-CDR3-a |
| 30 | TTGDQGTSHHMRSPMHWFPNW | Alternative Ab1 H-CDR3-b |
| 31 | MGAATTGDQGTSHHMRSPMHWFPN | Alternative Ab1 H-CDR3-c |
| 32 | NSSQSIVHSNGNTYLE | Alternative Ab1 L-CDR1-a |
| 33 | QSIVHSNGNTY | Alternative Ab1 L-CDR1-b |
| 34 | KVSNRFS | Alternative Ab1 L-CDR2-a |
| 35 | KVS | Alternative Ab1 L-CDR2-b |
| 36 | DQGSHVPWT | Alternative Ab1 L-CDR3-a |
| 37 | AQLQLVQSGPELRKPGETVKISCKASGYTFTDYSMH | Alternative Ab2 heavy |
| | WVKQVPGKVLKWMGWINTETGEPTYVDDFKGRFAF | |
| | SLETSASTAYLRLGGLLKNEDTATYFAPHQHQHHNFK | |
| | DVLEGNEQFLGGAAKGTVSSAKTTPPSVYPLAPGSA | |
| | AQTNSMVTLGCLVKGYFPEPVTVTWNSGSLSSGVHT | |
| | FPAVLQSDLYTLSSSVTVPSSTWPPCFMYPNVAHPA | |
| | SSTKVDKKIVPRQEYMKGNCTVPEVSSVFIFPPKPKD | |
| | VLTITLTKPVPGPWDVDISKDDPEVQFSWFVDDVEVH | |
| | TAQTQPREEQFNSTFRSVSELPPYHQDWLNGKEFKC | |
| | RVNSAAFPAPIEKTISKTKGRPKAPQVYTIPPPKEQMA | |
| | KDKVGMSMMSLSTTGGNSGSPVFGGEPHHNGQPAE | |
| | NYKNTQPIMDTDGSYFVYSKLNVQKSNWEAGNTFTC | |
| | SVLHEGLHNHHTEKSLSHSPGK | |
| 38 | DVVMTQTPLTLSVTYLEKGPAKCKSSQSLLDSDGQT | Alternative Ab2 light |
| | YLHWLLQRPGQSPKRLIYLVSKLGSGVPDRFTGSGS | |
| | GTDFTLKSIRVEAEDLGLYYSSGGEGAPDLLESQALT | |
| | EKAVVHNSSTLGPPSSSRYSQGNVSAVGVTYDGHTA | |
| | ITRKWKIDGSERQNGVLNSWTDQDSKDSTYSMSSTL | |
| | TLTKDEYTSPGFSVVATHKTSTSPIVKSFNRNEC | |
| 39 | AQLQLVQSGPELRKPGETVKISCKASGYTFTDYSMH | Alternative Ab2 VH |
| | WVKQVPGKVLKWMGWINTETGEPTYVDDFKGRFAF | |
| | SLETSASTAYLRLGGLLKNEDTATYFAPHQHQHHNFK | |
| | DVLEGNEQFLGGAAKGTVSS | |
| 40 | DVVMTQTPLTLSVTYLEKGPAKCKSSQSLLDSDGQT | Alternative Ab2 VL |
| | YLHWLLQRPGQSPKRLIYLVSKLGSGVPDRFTGSGS | |
| | GTDFTLKSIRVEAEDLGLYYSSGGEGAPDLLESQALT | |
| | EKA | |
| 41 | GYTFTDYSMH | Alternative Ab2 H-CDR1-a |
| 42 | GYTFTDYS | Alternative Ab2 H-CDR1-b |
| 43 | WINTETGEPTYVDDFKG | Alternative Ab2 H-CDR2-a |
| 44 | INTETGEP | Alternative Ab2 H-CDR2-b |
| 45 | QHQHHNFKDVLEGNEQF | Alternative Ab2 H-CDR3-a |
| 46 | KSSQSLLDSDGQTYLH | Alternative Ab2 L-CDR1-a |
| 47 | QSLLDSDGQTY | Alternative Ab2 L-CDR1-b |
| 48 | LVSKLGS | Alternative Ab2 L-CDR2-a |
| 49 | LVS | Alternative Ab2 L-CDR2-b |
| 50 | SGGEGAPDL | Alternative Ab2 L-CDR3-a |
| 51 | SSGGEGAPDL | Alternative Ab2 L-CDR3-b |
| 52 | QEVQLQQSGPELVRPGASVKNQSYLNLPDKDQLQAL | Alternative Ab3 heavy |
| | EKSLEKGNGRLNPYNGATNYNQNFKDKATLTVDKSS | |
| | STAYMELHSLTSEDSAVYYPHDGTRSFTTPPEAYWG | |
| | QGTLVTVSAAKTTPPSVYPLAPGCGDWYPVMNGLVK | |
| | HETPESVTVTWNSGSLSSSVHTFPALLQSGLYTMSS | |
| | SVTVPSSTWPSQTVTCMTVSVAHPASSTTVDKKLEP | |
| | SGPLSTLNWIGRLNPYDGATNYNQPAAKDKLEGGPS | |
| | VFIFPPNIKDVLMISPKLTVPEGVGLSVADLGISVTP | |
| | PGKVFNDYFSDFSGVYSAGLDKVVSTLPIQHQDWMSG | |
| | KYDLDFKVNNKDLPSPIERTISKIKGLVRAPQVYILP | |
| | PPAEQLSRKDMTHNKSIEWIGRLNPYNQTNYNQNFKDTA | |
| | PVLDSDGSYFIYSKLPGVNTSLTVKTDSFSPANRHEG | |
| | LKNYYLKKTISRSPGK | |
| 53 | YPLDTQAAFSNPVTLGTSASISVHSSKSLLYSNGLTYL | Alternative Ab3 light |
| | FWYLQRPGQSPQLILYQMSNLASGVPDRFSGSGSGT | |
| | DFTLRISRVEAEDVGPDCAQNLELPWTFGGGTKLEIK | |
| | RADAAPTVSIFPPSSEKYSQGNVSAVGVTYDGHTALT | |
| | RKDINVKWKIDGSERQNGVLNSWTDQDSKDSTYSMS | |
| | STLTLTKDEYERHPDEDCEATHKTSTSPIVKSFNRNE | |
| | C | |
| 54 | QEVQLQQSGPELVRPGASVKNQSYLNLPDKDQLQAL | Alternative Ab3 VH |
| | EKSLEKGNGRLNPYNGATNYNQNFKDKATLTVDKSS | |
| | STAYMELHSLTSEDSAVYYPHDGTRSFTTPPEAYWG | |
| | QGTLVTVSA | |
| 55 | YPLDTQAAFSNPVTLGTSASISVHSSKSLLYSNGLTYL | Alternative Ab3 VL |
| | FWYLQRPGQSPQLILYQMSNLASGVPDRFSGSGSGT | |
| | DFTLRISRVEAEDVGPDCAQNLELPWTFGGGTKLEIK | |
| 56 | LNLPDKDQLQALEK | Alternative Ab3 H-CDR1-a |
| 57 | NLPDKDQLQALE | Alternative Ab3 H-CDR1-b |
| 58 | PDKDQLQA | Alternative Ab3 H-CDR1-c |
| 59 | LNPYNGAT | Alternative Ab3 H-CDR2-a |
| 60 | HDGTRSFTTPPEAY | Alternative Ab3 H-CDR3-a |
| 61 | HSSKSLLYSNGLTYLF | Alternative Ab3 L-CDR1-a |
| 62 | KSLLYSNGLTY | Alternative Ab3 L-CDR1-b |
| 63 | QMSNLAS | Alternative Ab3 L-CDR2-a |
| 64 | QMS | Alternative Ab3 L-CDR2-b |
| 65 | AQNLELPWT | Alternative Ab3 L-CDR3-a |
| 66 | EVQLQQSGPELVKPGASVRIPCKTYGSTFTEYTMHW | Alternative Ab4 heavy |
| | VKQLGRKSLEWIGGLNPNSGNSRFSAGKFKDRATLT | |
| | VDKSSTTAYMELRSLTSEDSAVTPHTMSQPTHHHNY | |
| | GDYGYYAMDYWGQGTSVTVSSAKTTAPSVYPLAPV | |
| | CDAMCNYGVLGCKGYFPEPVTLTWNSGSLSSGVHT | |
| | FPAVLQSDLYTLSSSVTVTSSTWPSQSITCNVAHPAS | |
| | STKVDQLTKYSGEGDLALVKFSPNEQKNHLGEGWPV | |
| | RAVLPEIVTVVVDVSEDDPDVQISWFVNNVEVHTAQT | |
| | QTHREDYNSTLRVVSALPIQHQDWMSGKEFKCKVNN | |
| | KDLPAPIERTISKPKGSVRAPQVYVLPPPEEEMTKKY | |
| | SQGNVSAVGVTYDGHTALTRNGKTELNYKNTEPVLD | |
| | SDGSYFMYSKLRVEKKNWVERNSYSCSVVHEGLHN | |
| | HHTTKSFSRTPGK | |
| 67 | DIKMTQSPSSMYAFLGERVTITCKASQDIHRYLSWFQ | Alternative Ab4 light |
| | QKPGKSPKTLIYRANSLVDGVPSRFSGSGSGQDYSL | |
| | TISDTLLLELKRMKLQFAEFPLTFGAGTKLELKRADAA | |
| | PTVSIFPPSSEQLTSGGASVVTMGSSPSSMYPKDINV | |
| | KWKIDGSERQNGVLNSWTDQDSKDSTYSMSSTLTLT | |
| | KDEYERHNSYTCEATHKTSTSPIVKSFNRNEC | |
| 68 | EVQLQQSGPELVKPGASVRIPCKTYGSTFTEYTMHW | Alternative Ab4 VH |
| | VKQLGRKSLEWIGGLNPNSGNSRFSAGKFKDRATLT | |
| | VDKSSTTAYMELRSLTSEDSAVTPHTMSQPTHHHNY | |
| | GDYGYYAMDYWGQGTSVTVSS | |
| 69 | DIKMTQSPSSMYAFLGERVTITCKASQDIHRYLSWFQ | Alternative Ab4 VL |
| | QKPGKSPKTLIYRANSLVDGVPSRFSGSGSGQDYSL | |
| | TISDTLLLELKRMKLQFAEFPLTFGAGTKLELK | |
| 70 | GSTFTEYTMH | Alternative Ab4 H-CDR1-a |
| 71 | GSTFTEYT | Alternative Ab4 H-CDR1-b |
| 72 | GLNPNSGNSRFSAGKFKD | Alternative Ab4 H-CDR2-a |
| 73 | LNPNSGNSRFSAGKFKD | Alternative Ab4 H-CDR2-b |
| 74 | LNPNSGNSRFSAGKFKDRATLTVDKSSTT | Alternative Ab4 H-CDR2-c |
| 75 | THHHNYGDYGYYAMDY | Alternative Ab4 H-CDR3-a |
| 76 | KASQDIHRYLS | Alternative Ab4 L-CDR1-a |
| 77 | QDIHRY | Alternative Ab4 L-CDR1-b |
| 78 | RANSLVD | Alternative Ab4 L-CDR2-a |
| 79 | ANSLVD | Alternative Ab4 L-CDR2-b |
| 80 | RAN | Alternative Ab4 L-CDR2-c |
| 81 | QFAEFPLT | Alternative Ab4 L-CDR3-a |
| 82 | EVQLQQSGPELVKPGASVKISCKASGYSFTDPGDGM | Alternative Ab5 heavy |
| | HWVKFSSSLEWIGRINPYNQTKYNQKENDKASLTVD | |
| | KGWSGDCELLSGWGNTKVYMKSALEPPKSTTHHWK | |
| | MNSDGDYWGQGTTLRVSSAKHKMSVYPLAPGSAAQ | |
| | TNSMVTLGCLVKGYFPEPVTVTWNSGSLSSGVHTFP | |
| | AVLQSDLYTLSSSVTVPSSTWPESAFVCDTVSAPSAP | |
| | ASPVHTRDTLLLELKRPEVSSVFIFPPKPKDVLTITLTP | |
| | KVTSCMVVVDISKDDPEVQFSWFVDDVEVHTAQTQP | |
| | REEQFNSTFRSVSELPPYHQDWLNGKEFKCRVNSAA | |
| | FPAPIEKTISKTKGRPKAPQVYTIPPPKESSSTAYEML | |
| | HSLTSEDSAVYPEPSESSEKWQWNGQPAENYKNTQ | |
| | PIMDTDGSYFVYSKLNVQKSNWEAGNTFTCSVLHEG | |
| | LHNHHTEKSLSHSPGK | |
| 83 | DIVMTQAAFSNPVTLGTSASIAGAKEASKSLLYSNGIT | Alternative Ab5 light |
| | YLYWYLQKPGQSPQLLIYQMSNLASGVPDRESSSGS | |
| | GTDFTLRIPTVSSSYLNLAQNLELPWTFGGGTKLNFK | |
| | LREAVVTVKYSQGNVSAVGVTYDGHTALTRDINVKW | |
| | KIDGSERQNGVLNSWTDQDSKDSTYSMSSTLTLTKD | |
| | EYERHNSYTCEATHKTSTSPIVKSFNRNEC | |
| 84 | EVQLQQSGPELVKPGASVKISCKASGYSFTDPGDGM | Alternative Ab5 VH |
| | HWVKFSSSLEWIGRINPYNQTKYNQKENDKASLTVD | |
| | KGWSGDCELLSGWGNTKVYMKSALEPPKSTTHHWK | |
| | MNSDGDYWGQGTTLRVSS | |
| 85 | DIVMTQAAFSNPVTLGTSASIAGAKEASKSLLYSNGIT | Alternative Ab5 VL |
| | YLYWYLQKPGQSPQLLIYQMSNLASGVPDRESSSGS | |
| | GTDFTLRIPTVSSSYLNLAQNLELPWTFGGGTKLNFK | |
| 86 | GYSFTDPGDG | Alternative Ab5 H-CDR1-a |
| 87 | YSFTDPGD | Alternative Ab5 H-CDR1-b |
| 88 | SGYSFTDPGDGM | Alternative Ab5 H-CDR1-c |
| 89 | INPYNQT | Alternative Ab5 H-CDR2-a |
| 90 | NPYNQ | Alternative Ab5 H-CDR2-b |
| 91 | RINPYNQTK | Alternative Ab5 H-CDR2-c |
| 92 | KMNSDGDY | Alternative Ab5 H-CDR3-a |
| 93 | WKMNSDGDY | Alternative Ab5 H-CDR3-b |
| 94 | EASKSLLYSNGITYLY | Alternative Ab5 L-CDR1-a |
| 95 | KSLLYSNGITY | Alternative Ab5 L-CDR1-b |
| 96 | QMSNLAS | Alternative Ab5 L-CDR2-a |
| 97 | QMS | Alternative Ab5 L-CDR2-b |
| 98 | AQNLELPWT | Alternative Ab5 L-CDR3-a |
| 99 | AQNLELPW | Alternative Ab5 L-CDR3-b |
| 100 | QIQLVQSGPDLKKPGETVKISCKASGYSFTDFSVHWV | Alternative Ab6 heavy |
| | KQSPGMGIKWMGWINTETGEPTYADDFKGRFAFSLE | |
| | TSASTAYLQINYLKGGDDTATYFLHMHSTASNVAPET | |
| | VLPKMRHNRYDDDYWGQGTTLTVSSAKTTPPSVYPL | |
| | APGSAAQTNSMVTLGPTRVKFYGPEPVTVTWNSGSL | |
| | SSGVHTFPAVLQSDLYTLSSSVTVPSSTWPSEAFDS | |
| | WSVAQVASHTPTNLDVLEGNEQFLNAAKELTVAGDP | |
| | KSVRDTLLLELKRDFCVVVDISKDDPEVQFSWFVDDV | |
| | EVHTAQTQPREEQFNSTFRSVSELPIMHQDWLNGKE | |
| | FKCRVNSAAFPAPIEKTISKTKGRPKAPQVYTIPPPKE | |
| | QMAKDKVSLLPRSGTLAQDIPGRTPQYRSNGRENYK | |
| | NTQPPYDTDGSYFVYSKLNVQGSSRAGETYPEPEPT | |
| | PAPVPNYLASLSHSPGK | |
| 101 | DIVMTQSPSSLSVSAGEKVTMSCKSSQSLLNSGNGA | Alternative Ab6 light |
| | KNYLAWYQQKPGQPPKRLIYGASTRQSGVPDRFTGS | |
| | GSGTDFTLTISSVQAEDLAVYYCQNDHSYPLTFGAGT | |
| | KLELKRADAAPTVSIFPPSSEQLTKYSGEGDNQSYLN | |
| | LPKDSAGLSYIDGSERQNGVLNSWTDQDSKDSTYSM | |
| | SSTLTLTKDEYERHNSYTCEATHKTSTSPIVKSFNRN | |
| | EC | |
| 102 | QIQLVQSGPDLKKPGETVKISCKASGYSFTDFSVHWV | Alternative Ab6 VH |
| | KQSPGMGIKWMGWINTETGEPTYADDFKGRFAFSLE | |
| | TSASTAYLQINYLKGGDDTATYFLHMHSTASNVAPET | |
| | VLPKMRHNRYDDDYWGQGTTLTVSS | |
| 103 | DIVMTQSPSSLSVSAGEKVTMSCKSSQSLLNSGNGA | Alternative Ab6 VL |
| | KNYLAWYQQKPGQPPKRLIYGASTRQSGVPDRFTGS | |
| | GSGTDFTLTISSVQAEDLAVYYCQNDHSYPLTFGAGT | |
| | KLELK | |
| 104 | GYSFTDFSVH | Alternative Ab6 H-CDR1-a |
| 105 | GYSFTDFS | Alternative Ab6 H-CDR1-b |
| 106 | WINTETGEPTYADDFKG | Alternative Ab6 H-CDR2-a |
| 107 | INTETGEP | Alternative Ab6 H-CDR2-b |
| 108 | ETVLPKMRHNRYDDDY | Alternative Ab6 H-CDR3-a |
| 109 | VLPKMRHNRYDDDY | Alternative Ab6 H-CDR3-b |
| 110 | HMHSTASNVAPETVLPKMRHNRYDDDY | Alternative Ab6 H-CDR3-c |
| 111 | KSSQSLLNSGNGAKNYLA | Alternative Ab6 L-CDR1-a |
| 112 | QSLLNSGNGAKNY | Alternative Ab6 L-CDR1-b |
| 113 | GASTRQS | Alternative Ab6 L-CDR2-a |
| 114 | GAS | Alternative Ab6 L-CDR2-b |
| 115 | QNDHSYPLT | Alternative Ab6 L-CDR3-a |
| 116 | NDHSYPLT | Alternative Ab6 L-CDR3-b |
| 117 | QIQLVQSGPDLKNPGETVKISCKASGYSFTDFSVHWV | Alternative Ab7 heavy |
| | KQSPGLHLKAMMGWINTETGEPTYADDFKGRFAFSL | |
| | ETSASTAYLQINYLKNDDTATYFAKDFCREHMVTARN | |
| | RYDDDYWGQGTALTVSSAKTTAPSVYPLAPVPDFQT | |
| | SQATTTGWVKHETPEPVTLTWNSGSLSSGVHTFPAV | |
| | LQSDLVMTQSPVTLEKYSQGNVSAVGVTYDGHTALT | |
| | RKKIEPRGPTIKPCMMKNCQSPNLLGGPSVFIFPPKIK | |
| | DYDDDYWGKDGGRLVKGPDVQISWFVNNVEVFNDY | |
| | FSDFSGVYSAGLSRVVSALPIQHQDWMSGKEFKCKV | |
| | NNKDLPAPIERTISKPKGSVRAPQVYVLPPPEEEMTK | |
| | KQVEGETDIDVMTPHDQQKTELNYKNTEPVLDSDGS | |
| | YFMYSKLRVEKKNWVERNSYSCSVVHEGLHNHHTTK | |
| | SFSRTPGK | |
| 118 | DIVMTQSPSSLSVSAGEKVTMSCKSSQSLLNSGNQK | Alternative Ab7 light |
| | NYLAWYQQKPGQPPKRLIYGASTRQSGVPDRFTGS | |
| | GSGTDFTLTISSVQAEYDLAQSKCQNDHSYPLTFGAG | |
| | TKLELKRADAAPTVSIFPPSSEQLTSSTAAFTSSLNNF | |
| | YPKDINVKWKIDGSERQNGVLNSWTDQDSKDSTYSM | |
| | SSTLTLTKDEYERHVYFATHKTSTSPIVKSFNRNEC | |
| 119 | QIQLVQSGPDLKNPGETVKISCKASGYSFTDFSVHWV | Alternative Ab7 VH |
| | KQSPGLHLKAMMGWINTETGEPTYADDFKGRFAFSL | |
| | ETSASTAYLQINYLKNDDTATYFAKDFCREHMVTARN | |
| | RYDDDYWGQGTALTVSS | |
| 120 | DIVMTQSPSSLSVSAGEKVTMSCKSSQSLLNSGNQK | Alternative Ab7 VL |
| | NYLAWYQQKPGQPPKRLIYGASTRQSGVPDRFTGS | |
| | GSGTDFTLTISSVQAEYDLAQSKCQNDHSYPLTFGAG | |
| | TKLELK | |
| 121 | GYSFTDFSVH | Alternative Ab7 H-CDR1-a |
| 122 | GYSFTDFSVHW | Alternative Ab7 H-CDR1-b |
| 123 | GYSFTDFS | Alternative Ab7 H-CDR1-c |
| 124 | WINTETGEPTYADDFKG | Alternative Ab7 H-CDR2-a |
| 125 | WINTETGEPTYADDFKGR | Alternative Ab7 H-CDR2-b |
| 126 | INTETGEP | Alternative Ab7 H-CDR2-c |
| 127 | EHMVTARNRYDDDY | Alternative Ab7 H-CDR3-a |
| 128 | KDFCREHMVTARNRYDDDY | Alternative Ab7 H-CDR3-b |
| 129 | KSSQSLLNSGNQKNYLA | Alternative Ab7 L-CDR1-a |
| 130 | QSLLNSGNQKNY | Alternative Ab7 L-CDR1-b |
| 131 | GASTRQS | Alternative Ab7 L-CDR2-a |
| 132 | GAS | Alternative Ab7 L-CDR2-b |
| 133 | QNDHSYPLT | Alternative Ab7 L-CDR3-a |
| 134 | MGWSWIFLFLLSGTAGVLSEGQLQQSGPELVKPGAS | Ab1 heavy ngs |
| | VKISCKTSGYTFTEYTIHWVKQSHGKSLEWIGSIIPKN | |
| | AFTRYNQKFKGKAILTVDKSSSTAYMELRSLTSDDSA | |
| | VYYCARPGYDGHIWFPNWGQGTLVTVSAAKTTPPSV | |
| | YPLAPGCGDTTGSSVTLGCLVKGYFPESVTVTWNSG | |
| | SLSSSVHTFPALLQSGLYTMSSSVTVPSSTWPSQTVT | |
| | CSVAHPASSTTVDKKLEPSGPISTINPCPPCKECHKC | |
| | PAPNLEGGPSVFIFPPNIKDVLMISLTPKVTCVVVDVS | |
| | EDDPDVQISWFVNNVEVHTAQTQTHREDYNSTIRVV | |
| | STLPIQHQDWMSGKEFKCKVNNKDLPSPIERTISKIKG | |
| | LVRAPQVYILPPPAEQLSRKDVSLTCLVVGFNPGDISV | |
| | EWTSNGHTEENYKDTAPVLDSDGSYFIYSKLNMKTS | |
| | KWEKTDSFSCNVRHEGLKNYYLKKTISRSPGK | |
| 135 | MKLPVRLLVLMFWIPASSSDVLMTQTPLSLPVSLGDQ | Ab1 light ngs |
| | ASISCRSSQSIVHSNGNTYLEWYLQKPGQSPKLLIYK | |
| | VSNRFSGVPDRFSGSGSGTDFTLKISRVEAEDLGVYY | |
| | CFQGSHVPWTFGGGTKLEIKRADAAPTVSIFPPSSEQ | |
| | LTSGGASVVCFLNNFYPKDINVKWKIDGSERQNGVLN | |
| | SWTDQDSKDSTYSMSSTLTLTKDEYERHNSYTCEAT | |
| | HKTSTSPIVKSFNRNEC | |
| 136 | EGQLQQSGPELVKPGASVKISCKTSGYTFTEYTIHWV | Ab1 VH ngs |
| | KQSHGKSLEWIGSIIPKNAFTRYNQKFKGKAILTVDKS | |
| | SSTAYMELRSLTSDDSAVYYCARPGYDGHIWFPNWG | |
| | QGTLVTVSA | |
| 137 | DVLMTQTPLSLPVSLGDQASISCRSSQSIVHSNGNTY | Ab1 VL ngs |
| | LEWYLQKPGQSPKLLIYKVSNRFSGVPDRFSGSGSG | |
| | TDFTLKISRVEAEDLGVYYCFQGSHVPWTFGGGTKL | |
| | EIK | |
| 138 | GYTFTEYT | Ab1 H-CDR1 ngs imgt |
| 139 | EYTIH | Ab1 H-CDR1 ngs ka |
| 140 | IIPKNAFT | Ab1 H-CDR2 ngs imgt |
| 141 | SIIPKNAFTRYNQKFKG | Ab1 H-CDR2 ngs ka |
| 142 | ARPGYDGHIWFPN | Ab1 H-CDR3 ngs imgt |
| 143 | PGYDGHIWFPN | Ab1 H-CDR3 ngs ka |
| 144 | QSIVHSNGNTY | Ab1 L-CDR1 ngs imgt |
| 145 | RSSQSIVHSNGNTYLE | Ab1 L-CDR1 ngs ka |
| 146 | KVS | Ab1 L-CDR2 ngs imgt |
| 147 | KVSNRFS | Ab1 L-CDR2 ngs ka |
| 148 | FQGSHVPWT | Ab1 L-CDR3 ngs |
| 149 | MAWVWTLLFLMAAAQSIQAQIQLVQSGPELRKPGET | Ab2 heavy ngs |
| | VKISCKASGYTFTDYSMHWVKQVPGKVLKWMGWINT | |
| | ETGEPTYVDDFKGRFAFSLETSASTAYLRINILKNEDA | |
| | ATYFCATNFDYWGQGTTLTVSSAKTTPPSVYPLAPG | |
| | SAAQTNSMVTLGCLVKGYFPEPVTVTWNSGSLSSGV | |
| | HTFPAVLQSDLYTLSSSVTVPSSTWPSETVTCNVAHP | |
| | ASSTKVDKKIVPRDCGCKPCICTVPEVSSVFIFPPKPK | |
| | DVLTITLTPKVTCVVVDISKDDPEVQFSWFVDDVEVH | |
| | TAQTQPREEQFNSTFRSVSELPIMHQDWLNGKEFKC | |
| | RVNSAAFPAPIEKTISKTKGRPKAPQVYTIPPPKEQMA | |
| | KDKVSLTCMITDFFPEDITVEWQWNGQPAENYKNTQ | |
| | PIMDTDGSYFVYSKLNVQKSNWEAGNTFTCSVLHEG | |
| | LHNHHTEKSLSHSPGK | |
| 150 | MSPAQFLFLLVLWIRETNGDVVMTQTPLTLSVSIGQP | Ab2 light ngs |
| | ASISCKSSQSLLDSDGQTYLHWLLQRPGQSPKRLIYL | |
| | VSKLGSGVPDRFTGSGSGTDFTLKISRVEAEDLGLYY | |
| | CWQGTHFPRTFGGGTKLEIKRADAAPTVSIFPPSSEQ | |
| | LTSGGASVVCFLNNFYPKDINVKWKIDGSERQNGVLN | |
| | SWTDQDSKDSTYSMSSTLTLTKDEYERHNSYTCEAT | |
| | HKTSTSPIVKSFNRNEC | |
| 151 | QIQLVQSGPELRKPGETVKISCKASGYTFTDYSMHW | Ab2 VH ngs |
| | VKQVPGKVLKWMGWINTETGEPTYVDDFKGRFAFSL | |
| | ETSASTAYLRINILKNEDAATYFCATNFDYWGQGTTLT | |
| | VSS | |
| 152 | DVVMTQTPLTLSVSIGQPASISCKSSQSLLDSDGQTY | Ab2 VL ngs |
| | LHWLLQRPGQSPKRLIYLVSKLGSGVPDRFTGSGSG | |
| | TDFTLKISRVEAEDLGLYYCWQGTHFPRTFGGGTKLE | |
| | IK | |
| 153 | DYSMH | Ab2 H-CDR1 ngs ka |
| 154 | GYTFTDY | Ab2 H-CDR1 ngs imgt |
| 155 | WINTETGEPTYVDDFKG | Ab2 H-CDR2 ngs ka |
| 156 | INTETGEP | Ab2 H-CDR2 ngs imgt |
| 157 | NFDY | Ab2 H-CDR3 ngs ka |
| 158 | ATNFDY | Ab2 H-CDR3 ngs imgt |
| 159 | KSSQSLLDSDGQTYLH | Ab2 L-CDR1 ngs ka |
| 160 | QSLLDSDGQTY | Ab2 L-CDR1 ngs imgt |
| 161 | LVSKLGS | Ab2 L-CDR2 ngs ka |
| 162 | LVS | Ab2 L-CDR2 ngs imgt |
| 163 | WQGTHFPRT | Ab2 L-CDR3 ngs |
| 164 | MGWSWIFLFLLSGTAGVLSEVQLQQSGPELVRPGAS | Ab3 heavy ngs |
| | VKISCKASGYSFTDYFMHWMKQSHVKSLEWIGRLNP | |
| | YNGATNYNQNFKDKATLTVDKSSSTAYMELHSLTSE | |
| | DSAVYYCLSFTTPPEAYWGQGTLVTVSAAKTTPPSV | |
| | YPLAPGCGDTTGSSVTLGCLVKGYFPESVTVTWNSG | |
| | SLSSSVHTFPALLQSGLYTMSSSVTVPSSTWPSQTVT | |
| | CSVAHPASSTTVDKKLEPSGPISTINPCPPCKECHKC | |
| | PAPNLEGGPSVFIFPPNIKDVLMISLTPKVTCVVVDVS | |
| | EDDPDVQISWFVNNVEVHTAQTQTHREDYNSTIRVV | |
| | STLPIQHQDWMSGKEFKCKVNNKDLPSPIERTISKIKG | |
| | LVRAPQVYILPPPAEQLSRKDVSLTCLVVGFNPGDISV | |
| | EWTSNGHTEENYKDTAPVLDSDGSYFIYSKLNMKTS | |
| | KWEKTDSFSCNVRHEGLKNYYLKKTISRSPGK | |
| 165 | MRFSAQLLGLLVLWIPGSTAEIVMTQAAFSNPVTLGT | Ab3 light ngs |
| | SASISCRSNKSLLYSNGITYLFWYLQRPGQSPQLLIYQ | |
| | MSNLASGVPDRFSGSGSGTDFTLRISRVEAEDVGVY | |
| | YCAQNLELPWTFGGGTKLEIKRADAAPTVSIFPPSSE | |
| | QLTSGGASVVCFLNNFYPKDINVKWKIDGSERQNGV | |
| | LNSWTDQDSKDSTYSMSSTLTLTKDEYERHNSYTCE | |
| | ATHKTSTSPIVKSFNRNEC | |
| 166 | EVQLQQSGPELVRPGASVKISCKASGYSFTDYFMHW | Ab3 VH ngs |
| | MKQSHVKSLEWIGRLNPYNGATNYNQNFKDKATLTV | |
| | DKSSSTAYMELHSLTSEDSAVYYCLSFTTPPEAYWG | |
| | QGTLVTVSA | |
| 167 | EIVMTQAAFSNPVTLGTSASISCRSNKSLLYSNGITYL | Ab3 VL ngs |
| | FWYLQRPGQSPQLLIYQMSNLASGVPDRFSGSGSGT | |
| | DFTLRISRVEAEDVGVYYCAQNLELPWTFGGGTKLEI | |
| | K | |
| 168 | DYFMH | Ab3 H-CDR1 ngs ka |
| 169 | GYSFTDYF | Ab3 H-CDR1 ngs imgt |
| 170 | RLNPYNGATNYNQNFKD | Ab3 H-CDR2 ngs ka |
| 171 | LNPYNGAT | Ab3 H-CDR2 ngs imgt |
| 172 | FTTPPEAY | Ab3 H-CDR3 ngs ka |
| 173 | LSFTTPPEAY | Ab3 H-CDR3 ngs imgt |
| 174 | RSNKSLLYSNGITYLF | Ab3 L-CDR1 ngs ka |
| 175 | KSLLYSNGITY | Ab3 L-CDR1 ngs imgt |
| 176 | QMSNLAS | Ab3 L-CDR2 ngs ka |
| 177 | QMS | Ab3 L-CDR2 ngs imgt |
| 178 | AQNLELPWT | Ab3 L-CDR3 ngs |
| 179 | MGWSWIFLFLLSGTAGVLSEVQLQQSGPELVKPGAS | Ab4 heavy ngs |
| | VRISCKTSGYTFTEYTMHWVKQRLGKSLEWIGGINPN | |
| | SGNSRFSQKFKDRATLTVDKSSTTAYMELRSLTSEDS | |
| | AVYFCTRNYGDYGYFAMDYWGQGTSVTVSSAKTTA | |
| | PSVYPLAPVCGDTTGSSVTLGCLVKGYFPEPVTLTW | |
| | NSGSLSSGVHTFPAVLQSDLYTLSSSVTVPSSTWPS | |
| | QTVTCSVAHPASSTTVDKKLEPSGPISTINPCPPCKE | |
| | CHKCPAPNLEGGPSVFIFPPKIKDVLMISLSPIVTCVVV | |
| | DVSEDDPDVQISWFVNNVEVHTAQTQTHREDYNSTL | |
| | RVVSALPIQHQDWMSGKEFKCKVNNKDLPAPIERTIS | |
| | KPKGSVRAPQVYVLPPPEEEMTKKQVTLTCMVTDFM | |
| | PEDIYVEWTNNGKTELNYKNTEPVLDSDGSYFMYSK | |
| | LRVEKKNWVERNSYSCSVVHEGLHNHHTTKSFSRTP | |
| | GK | |
| 180 | MRTPAQFLGILLLWFPGIKCDIKMTQSPSSMYAFLGE | Ab4 light ngs |
| | RVTIACKASQDIHRYLSWFQQKPGKSPKTLIYRANSL | |
| | VDGVPSRFSGSGSGQDYSLTISSLEYEDVGIYYCLQF | |
| | AEFPLTFGAGTKLELKRADAAPTVSIFPPSSEQLTSG | |
| | GASVVCFLNNFYPKDINVKWKIDGSERQNGVLNSWT | |
| | DQDSKDSTYSMSSTLTLTKDEYERHNSYTCEATHKT | |
| | STSPIVKSFNRNEC | |
| 181 | EVQLQQSGPELVKPGASVRISCKTSGYTFTEYTMHW | Ab4 VH ngs |
| | VKQRLGKSLEWIGGINPNSGNSRFSQKFKDRATLTVD | |
| | KSSTTAYMELRSLTSEDSAVYFCTRNYGDYGYFAMD | |
| | YWGQGTSVTVSS | |
| 182 | DIKMTQSPSSMYAFLGERVTIACKASQDIHRYLSWFQ | Ab4 VL ngs |
| | QKPGKSPKTLIYRANSLVDGVPSRFSGSGSGQDYSL | |
| | TISSLEYEDVGIYYCLQFAEFPLTFGAGTKLELK | |
| 183 | EYTMH | Ab4 H-CDR1 ngs ka |
| 184 | GYTFTEYT | Ab4 H-CDR1 ngs imgt |
| 185 | GINPNSGNSRFSQKFKD | Ab4 H-CDR2 ngs ka |
| 186 | INPNSGNS | Ab4 H-CDR2 ngs imgt |
| 187 | YGYFAMDY | Ab4 H-CDR3 ngs ka |
| 188 | TRNYGDYGYFAMDY | Ab4 H-CDR3 ngs imgt |
| 189 | KASQDIHRYLSWFQQKP | Ab4 L-CDR1 ngs ka |
| 190 | QDIHRY | Ab4 L-CDR1 ngs imgt |
| 191 | RANSLVD | Ab4 L-CDR2 ngs ka |
| 192 | RAN | Ab4 L-CDR2 ngs imgt |
| 193 | LQFAEFPLT | Ab4 L-CDR3 ngs |
| 194 | MGWSWIFLFLLSGTAGVLSEVQLQQSGPELVKPGAS | Ab5 heavy ngs |
| | VKISCKASGYSFTDYYMHWVKQSHVKSLEWIGRINPY | |
| | NGATKYNQNFKDKASLTVDKSSSTAYMELHSLTSED | |
| | SAVYYCTRRYDNDDGDYWGQGTTLRVSSAKTTPPS | |
| | VYPLAPGSAAQTNSMVTLGCLVKGYFPEPVTVTWNS | |
| | GSLSSGVHTFPAVLQSDLYTLSSSVTVPSSTWPSETV | |
| | TCNVAHPASSTKVDKKIVPRDCGCKPCICTVPEVSSV | |
| | FIFPPKPKDVLTITLTPKVTCVVVDISKDDPEVQFSWF | |
| | VDDVEVHTAQTQPREEQFNSTFRSVSELPIMHQDWL | |
| | NGKEFKCRVNSAAFPAPIEKTISKTKGRPKAPQVYTIP | |
| | PPKEQMAKDKVSLTCMITDFFPEDITVEWQWNGQPA | |
| | ENYKNTQPIMDTDGSYFVYSKLNVQKSNWEAGNTFT | |
| | CSVLHEGLHNHHTEKSLSHSPGK | |
| 195 | MRFSAQLLGLLVLWIPGSTADIVMTQAAFSNPVTLGT | Ab5 light ngs |
| | SASISCRSSKSLLYSNGITYLYWYLQKPGQSPQLLIYQ | |
| | MSNLASGVPDRFSSSGSGTDFTLRISRVEAEDVGVY | |
| | YCAQNLELPWTFGGGTKLEIKRADAAPTVSIFPPSSE | |
| | QLTSGGASVVCFLNNFYPKDINVKWKIDGSERQNGV | |
| | LNSWTDQDSKDSTYSMSSTLTLTKDEYERHNSYTCE | |
| | ATHKTSTSPIVKSFNRNEC | |
| 196 | EVQLQQSGPELVKPGASVKISCKASGYSFTDYYMHW | Ab5 VH ngs |
| | VKQSHVKSLEWIGRINPYNGATKYNQNFKDKASLTVD | |
| | KSSSTAYMELHSLTSEDSAVYYCTRRYDNDDGDYW | |
| | GQGTTLRVSS | |
| 197 | DIVMTQAAFSNPVTLGTSASISCRSSKSLLYSNGITYL | Ab5 VL ngs |
| | YWYLQKPGQSPQLLIYQMSNLASGVPDRESSSGSGT | |
| | DFTLRISRVEAEDVGVYYCAQNLELPWTFGGGTKLEI | |
| | K | |
| 198 | DYYMH | Ab5 H-CDR1 ngs ka |
| 199 | GYSFTDYY | Ab5 H-CDR1 ngs imgt |
| 200 | RINPYNGATKYNQNFKD | Ab5 H-CDR2 ngs ka |
| 201 | INPYNGAT | Ab5 H-CDR2 ngs imgt |
| 202 | RYDNDDGD | Ab5 H-CDR3 ngs ka |
| 203 | TRRYDNDDGDY | Ab5 H-CDR3 ngs imgt |
| 204 | RSSKSLLYSNGITYLY | Ab5 L-CDR1 ngs ka |
| 205 | KSLLYSNGITY | Ab5 L-CDR1 ngs imgt |
| 206 | QMSNLAS | Ab5 L-CDR2 ngs ka |
| 207 | QMS | Ab5 L-CDR2 ngs imgt |
| 208 | AQNLELPWT | Ab5 L-CDR3 ngs |
| 209 | MAWVWTLLFLMAAAQRIQAQIQLVQSGPDLKKPGET | Ab6 heavy ngs |
| | VKISCKASGYSFTDFSVHWVKQSPGMGLKWMGWIN | |
| | TETGEPTYADDFKGRFAFSLETSASTAYLQINYLKND | |
| | DTATYFCARGGRYDDDYWGQGTTLTVSSAKTTPPSV | |
| | YPLAPGSAAQTNSMVTLGCLVKGYFPEPVTVTWNSG | |
| | SLSSGVHTFPAVLQSDLYTLSSSVTVPSSTWPSETVT | |
| | CNVAHPASSTKVDKKIVPRDCGCKPCICTVPEVSSVFI | |
| | FPPKPKDVLTITLTPKVTCVVVDISKDDPEVQFSWFVD | |
| | DVEVHTAQTQPREEQFNSTFRSVSELPIMHQDWLNG | |
| | KEFKCRVNSAAFPAPIEKTISKTKGRPKAPQVYTIPPP | |
| | KEQMAKDKVSLTCMITDFFPEDITVEWQWNGQPAEN | |
| | YKNTQPIMDTDGSYFVYSKLNVQKSNWEAGNTFTCS | |
| | VLHEGLHNHHTEKSLSHSPGK | |
| 210 | MESQTQVLISLLFWVSGTCGDIVMTQSPSSLSVSAGE | Ab6 light ngs |
| | KVTMSCKSSQSLLNSGNQKNYLAWYQQKPGQPPKR | |
| | LIYGASTRQSGVPDRFTGSGSGTDFTLTISSVQAEDL | |
| | AVYYCQNDHSYPLTFGAGTKLELKRADAAPTVSIFPP | |
| | SSEQLTSGGASVVCFLNNFYPKDINVKWKIDGSERQ | |
| | NGVLNSWTDQDSKDSTYSMSSTLTLTKDEYERHNSY | |
| | TCEATHKTSTSPIVKSFNRNEC | |
| 211 | QIQLVQSGPDLKKPGETVKISCKASGYSFTDFSVHWV | Ab6 VH ngs |
| | KQSPGMGLKWMGWINTETGEPTYADDFKGRFAFSL | |
| | ETSASTAYLQINYLKNDDTATYFCARGGRYDDDYWG | |
| | QGTTLTVSS | |
| 212 | DIVMTQSPSSLSVSAGEKVTMSCKSSQSLLNSGNQK | Ab6 VL ngs |
| | NYLAWYQQKPGQPPKRLIYGASTRQSGVPDRFTGS | |
| | GSGTDFTLTISSVQAEDLAVYYCQNDHSYPLTFGAGT | |
| | KLELK | |
| 213 | DFSVH | Ab6 H-CDR1 ngs ka |
| 214 | GYSFTDFS | Ab6 H-CDR1 ngs imgt |
| 215 | WINTETGEPTYADDFKG | Ab6 H-CDR2 ngs ka |
| 216 | INTETGEP | Ab6 H-CDR2 ngs imgt |
| 217 | GGRYDDDY | Ab6 H-CDR3 ngs ka |
| 218 | ARGGRYDDDY | Ab6 H-CDR3 ngs imgt |
| 219 | KSSQSLLNSGNQKNYLA | Ab6 L-CDR1 ngs ka |
| 220 | QSLLNSGNQKNY | Ab6 L-CDR1 ngs imgt |
| 221 | GASTRQS | Ab6 L-CDR2 ngs ka |
| 222 | GAS | Ab6 L-CDR2 ngs imgt |
| 223 | QNDHSYPLT | Ab6 L-CDR3 ngs |
| 224 | MAWVWTLLFLMAAAQRIQAQIQLVQSGPDLKNPGET | Ab7 heavy ngs |
| | VKISCKASGYSFTDFSVHWVKQSPGIHLKWMGWINT | |
| | ETGEPTYADDFKGRFAFSLETSASTAYLQINYLKNDD | |
| | TATYFCARGGRYDDDYWGQGTALTVSSAKTTAPSVY | |
| | PLAPVCGDTTGSSVTLGCLVKGYFPEPVTLTWNSGS | |
| | LSSGVHTFPAVLQSDLYTLSSSVTVTSSTWPSQSITC | |
| | NVAHPASSTKVDKKIEPRGPTIKPCPPCKCPAPNLLG | |
| | GPSVFIFPPKIKDVLMISLSPIVTCVVVDVSEDDPDVQI | |
| | SWFVNNVEVHTAQTQTHREDYNSTLRVVSALPIQHQ | |
| | DWMSGKEFKCKVNNKDLPAPIERTISKPKGSVRAPQ | |
| | VYVLPPPEEEMTKKQVTLTCMVTDFMPEDIYVEWTN | |
| | NGKTELNYKNTEPVLDSDGSYFMYSKLRVEKKNWVE | |
| | RNSYSCSVVHEGLHNHHTTKSFSRTPGK | |
| 225 | MESQTQVLISLLFWVSGTCGDIVMTQSPSSLSVSAGE | Ab7 light ngs |
| | KVTMSCKSSQSLLNSGNQKNYLAWYQQKPGQPPKR | |
| | LIYGASTRQSGVPDRFTGSGSGTDFTLTISSVQAEDL | |
| | AVYYCQNDHSYPLTFGAGTKLELKRADAAPTVSIFPP | |
| | SSEQLTSGGASVVCFLNNFYPKDINVKWKIDGSERQ | |
| | NGVLNSWTDQDSKDSTYSMSSTLTLTKDEYERHNSY | |
| | TCEATHKTSTSPIVKSFNRNEC | |
| 226 | QIQLVQSGPDLKNPGETVKISCKASGYSFTDFSVHWV | Ab7 VH ngs |
| | KQSPGIHLKWMGWINTETGEPTYADDFKGRFAFSLE | |
| | TSASTAYLQINYLKNDDTATYFCARGGRYDDDYWGQ | |
| | GTALTVSS | |
| 227 | DIVMTQSPSSLSVSAGEKVTMSCKSSQSLLNSGNQK | Ab7 VL ngs |
| | NYLAWYQQKPGQPPKRLIYGASTRQSGVPDRFTGS | |
| | GSGTDFTLTISSVQAEDLAVYYCQNDHSYPLTFGAGT | |
| | KLELK | |
| 228 | DFSVH | Ab7 H-CDR1 ngs ka |
| 229 | GYSFTDFS | Ab7 H-CDR1 ngs imgt |
| 230 | WINTETGEPTYADDFKG | Ab7 H-CDR2 ngs ka |
| 231 | INTETGEP | Ab7 H-CDR2 ngs imgt |
| 232 | GGRYDDDY | Ab7 H-CDR3 ngs ka |
| 233 | ARGGRYDDDY | Ab7 H-CDR3 ngs imgt |
| 234 | KSSQSLLNSGNQKNYLA | Ab7 L-CDR1 ngs ka |
| 235 | QSLLNSGNQKNY | Ab7 L-CDR1 ngs imgt |
| 236 | GASTRQS | Ab7 L-CDR2 ngs ka |
| 237 | GAS | Ab7 L-CDR2 ngs imgt |
| 238 | QNDHSYPLT | Ab7 L-CDR3 ngs |
| 239 | XGYTFTEYTXXX | Ab1 H-CDR1 (general 1) |
| 240 | GYTFTEYT | Ab1 H-CDR1 (general 2) |
| 241 | XXXXPKNXXXXXXXXXX | Ab1 H-CDR2 (general 1) |
| 242 | XXPKNX | Ab1 H-CDR2 (general 2) |
| 243 | XXXXXXXXXXXXXXXXXXXXWFPNX | Ab1 H-CDR3 (general 1) |
| 244 | XXXXXXXWFPN | Ab1 H-CDR3 (general 2) |
| 245 | XXXQSIVHSNGNTYXX | Ab1 L-CDR1 (general) |
| 246 | KVSXXXX | Ab1 L-CDR2 (general) |
| 247 | XQGSHVPWT | Ab1 L-CDR3 (general) |
| 248 | GYTFTDYXXX | Ab2 H-CDR1 (general 1) |
| 249 | XINTETGEPXXXXXXXX | Ab2 H-CDR2 (general) |
| 250 | XXXXXNFXXXXXXXXXX | Ab2 H-CDR3 (general) |
| 251 | XXXQSLLDSDGQTYXX | Ab2 L-CDR1 (general) |
| 252 | LVSXXXX | Ab2 L-CDR2 (general) |
| 253 | XXXGXXXPXX | Ab2 L-CDR3 (general) |
| 254 | XXXXDXXXXXXXXX | Ab3 H-CDR1 (general) |
| 255 | XLNPYNGATXXXXXXXX | Ab3 H-CDR2 (general) |
| 256 | XXXXXXFTTPPEAY | Ab3 H-CDR3 (general) |
| 257 | XXXKSLLYSNGXTYXX | Ab3 L-CDR1 (general) |
| 258 | QMSXXXX | Ab3 L-CDR2 (general) |
| 259 | XAQNLELPWTX | Ab3 L-CDR3 (general) |
| 260 | XXXXXEYTXX | Ab4 H-CDR1 (general) |
| 261 | XXNPNSGNSRFSXXXXXXXXXXXXXXXXXX | Ab4 H-CDR2 (general 1) |
| 262 | NPNSGNSRFS | Ab4 H-CDR2 (general 2) |
| 263 | XXXXNYGDYGYXAMDY | Ab4 H-CDR3 (general 1) |
| 264 | YGYXAMDY | Ab4 H-CDR3 (general 2) |
| 265 | XXXQDIHRYXX | Ab4 L-CDR1 (general 1) |
| 266 | KASQDIHRYLSXXXXXX | Ab4 L-CDR1 (general 2) |
| 267 | XANXXXX | Ab4 L-CDR2 (general) |
| 268 | XQFAEFPLT | Ab4 L-CDR3 (general) |
| 269 | XXXXXXDXXXXX | Ab5 H-CDR1 (general 1) |
| 270 | YSFTD | Ab5 H-CDR1 (general 2) |
| 271 | XXXXXXGATXXXXXXXXX | Ab5 H-CDR2 (general 1) |
| 272 | INPYN | Ab5 H-CDR2 (general 2) |
| 273 | XXXXXNXDGDX | Ab5 H-CDR3 (general 1) |
| 274 | NXDGD | Ab5 H-CDR3 (general 2) |
| 275 | XXXKSLLYSNGITYXX | Ab5 L-CDR1 (general 1) |
| 276 | KSLLYSNGITY | Ab5 L-CDR1 (general 2) |
| 277 | QMSXXXX | Ab5 L-CDR2 (general) |
| 278 | XAQNLELPWTX | Ab5 L-CDR3 (general) |
| 279 | XXXXXDFSXX | Ab6 H-CDR1 (general) |
| 280 | XINTETGEPXXXXXXXX | Ab6 H-CDR2 (general) |
| 28 | XXGGRYDDDY | Ab6 H-CDR3 (general 1) |
| 282 | XXXXRYDDDY | Ab6 H-CDR3 (general 2) |
| 283 | XXXQSLLNSGNQKNYXX | Ab6 L-CDR1 (general 1) |
| 284 | QSLLNSGN | Ab6 L-CDR1 (general 2) |
| 285 | GASXXXX | Ab6 L-CDR2 (general) |
| 286 | XXNDHSYPLTX | Ab6 L-CDR3 (general) |
| 287 | XXXXXDFSXX | Ab7 H-CDR1 (general) |
| 288 | XINTETGEPXXXXXXXX | Ab7 H-CDR2 (general) |
| 289 | XXGGRYDDDY | Ab7 H-CDR3 (general 1) |
| 290 | XXXYDDDY | Ab7 H-CDR3 (general 2) |
| 291 | XXXQSLLNSGNQKNYXX | Ab7 L-CDR1 (general) |
| 292 | GASXXXX | Ab7 L-CDR2 (general) |
| 293 | XQNDHSYPLTX | Ab7 L-CDR3 (general) |

### FIGURES

The invention is demonstrated by way of the example through the figures disclosed herein. The figures provided represent particular, non-limiting embodiments and are not intended to limit the scope of the invention.

### SHORT DESCRIPTION OF THE FIGURES

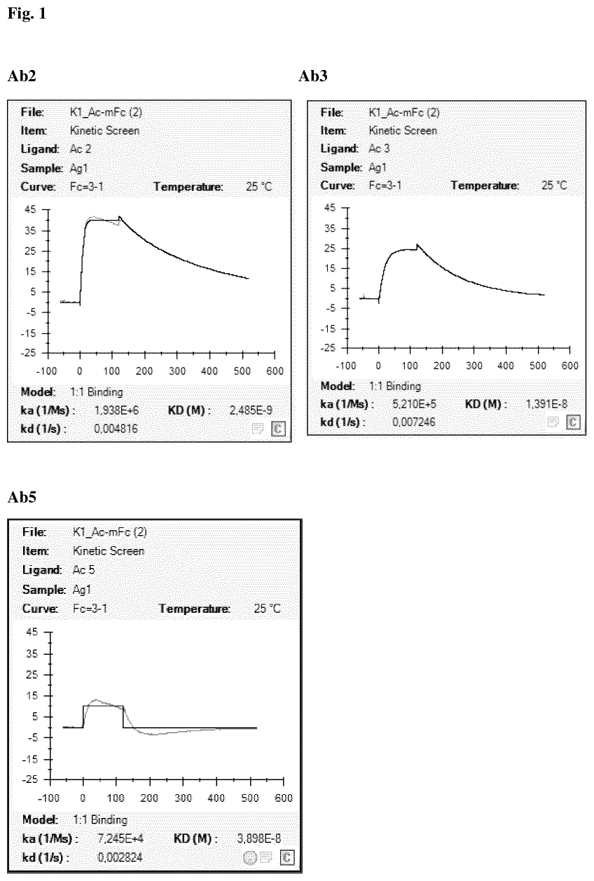

FIG. 1: SPR measurements against MR-proADM antigen 1.

FIG. 2: SPR measurements against MR-proADM antigen 2.

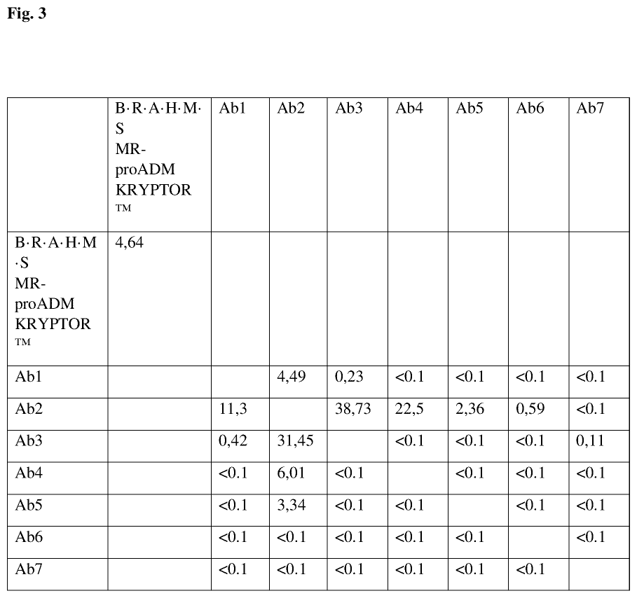

FIG. 3: Summary of MR-proADM measurements.

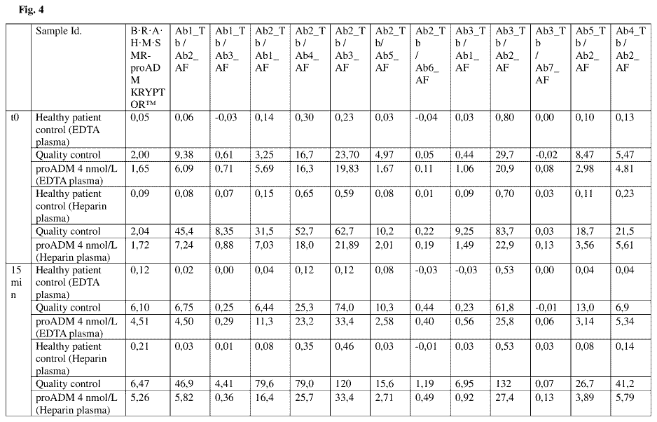

FIG. 4: Data from MR-proADM measurements.

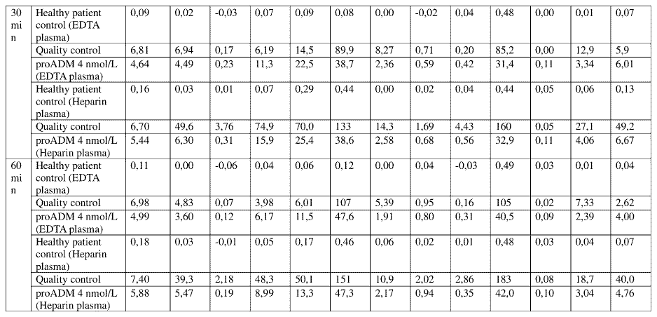

FIG. 5: Data from MR-proADM measurements.

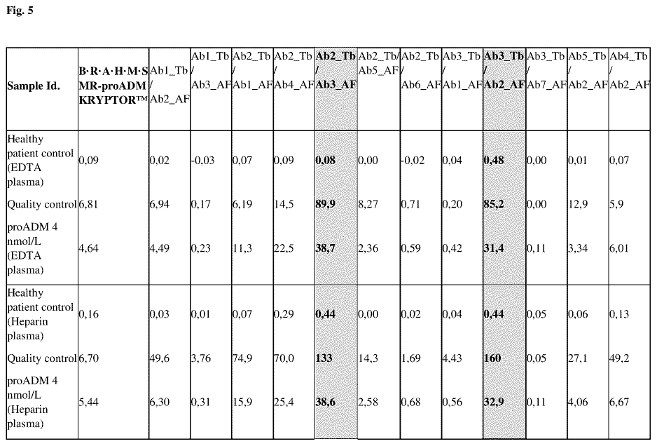

FIG. 6: Comparative quantitative MR-proADM measurements for two assays.

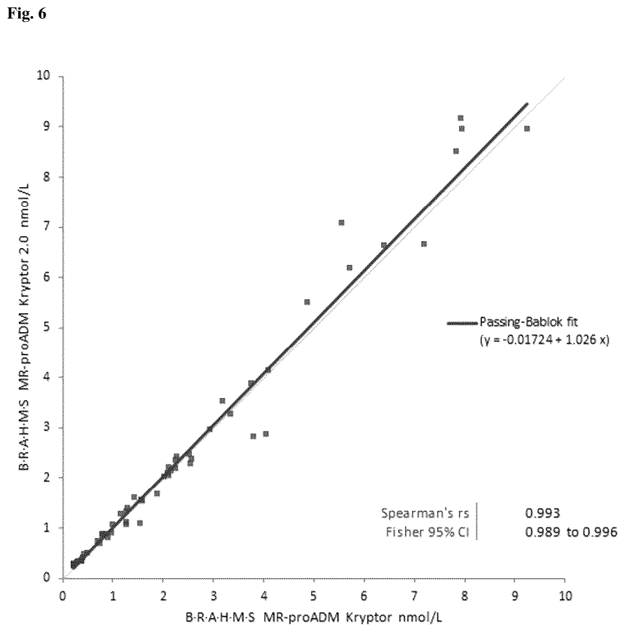

FIG. 7: Comparative quantitative MR-proADM measurements for two assays.

### DETAILED DESCRIPTION OF THE FIGURES

FIG. 1: SPR measurements against MR-proADM antigen 1. Surface plasmon resonance was employed to determine affinity of the antibodies Ab2, Ab3 and Ab5 against the MR-proADM antigen.

FIG. 2: SPR measurements against MR-proADM antigen 2. Surface plasmon resonance was employed to determine affinity of the antibodies Ab2, Ab3 and Ab5 against the MR-proADM antigen.

FIG. 3: Summary of MR-proADM measurements. The inventive antibodies were employed in an immunoluminometric assay to determine function and make comparisons to established methods including the B⋅R⋅A⋅H⋅M⋅S MR-proADM KRYPTOR assay, based on polyclonal antibodies against MR-proADM.

FIG. 4: Data from MR-proADM measurements. Summary of MR-proADM measurements. The inventive antibodies were employed in an immunoluminometric assay to determine function and make comparisons to established methods including the B⋅R⋅A⋅H⋅M⋅S MR-proADM KRYPTOR assay, based on polyclonal antibodies against MR-proADM. Samples were provided from healthy patients (low MR-proADM), quality control samples were run in parallel, and samples with 4 nmol/L MR-proADM, either in EDTA or Heparin-treated plasma samples, were analysed using different combinations of the inventive antibodies. Antibodies were labelled appropriately (Tb=Terbium donor. AF=Alexa Fluorophore acceptor) and analysed in in an immunoluminometric assay system.

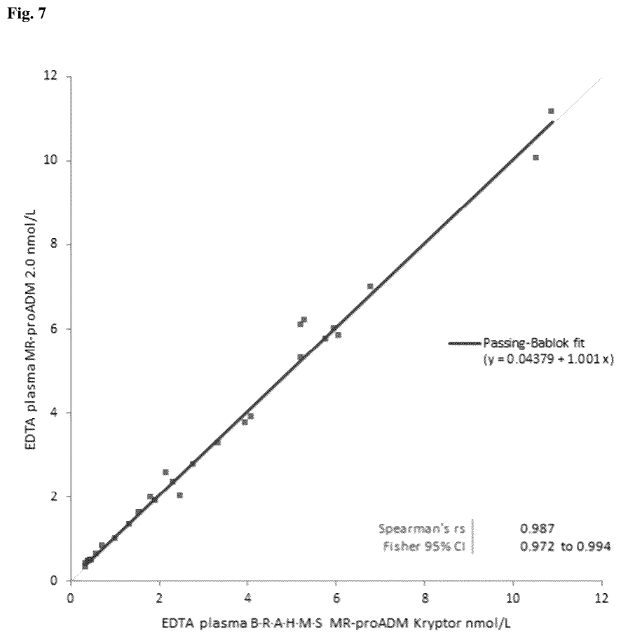

FIG. 5: Shows an excerpt from FIG. 11 at the 30-min time point, identifying the beneficial combination of Ab2 and Ab3 in comparison to the established assay (B⋅R⋅A⋅H⋅M⋅S MR-proADM KRYPTOR™ assay) based on labelled polyclonal antibodies.

FIG. 6: Method comparisons were conducted between the established B⋅R⋅A⋅H⋅M⋅S MR-proADM assay and the new (inventive) assay “MR-proADM 2.0 mAb”, using the Ac2-Tb/Ab3-AF assay format, in two separate comparisons, namely using 57 EDTA plasma samples (KG0007). Quantitative MR-proADM measurements were determined for individual samples to compare performance of the two assays.

FIG. 7: Method comparisons were conducted between the established B⋅R⋅A⋅H⋅M⋅S MR-proADM assay and the new (inventive) assay “MR-proADM 2.0 mAb”, using the Ac2-Tb/Ab3-AF assay format, in two separate comparisons, namely using 29 EDTA plasma samples (KG0166). Quantitative MR-proADM measurements were determined for individual samples to compare performance of the two assays.

### EXAMPLES

The invention is demonstrated through the examples disclosed herein. The examples provided represent particular embodiments and are not intended to limit the scope of the invention. The examples are to be considered as providing a non-limiting illustration and technical support for carrying out the invention.

The examples below present characterization of various monoclonal antibodies obtained from immunizations in mice using fragments of MR-proADM. The examples further demonstrate sequencing of antibodies and relevant variable domain sequences of the antibodies. The examples further demonstrate use of the antibodies in determining MR-proADM in plasma samples using an immunoluminometric assay.

### Immunization in Mice:

Mice were immunized according to established protocols. For example, mice are immunized by giving biweekly injections of a purified antigen, for example those according to SEQ ID NO 5-18, preferably SEQ ID NO 6, 8, 10, 12, 14, 16 or 18.

For mice, soluble protein antigen is used, typically a dose of 50-100 μg at each immunization is employed. Adjuvants (for example Freund's, Ribi, Hunter's TiterMax, ImmunEasy, or Alum) are mixed with the immunizing antigen for the first two immunizations. Complete Freund's adjuvant is used with the first immunization. Subsequent immunizations are performed in phosphate-buffered saline (PBS) or normal saline, with or without Incomplete Freund's adjuvant. Over the course of a 6-wk immunization schedule, each animal can receive a total of six injections. Once a suitable antibody titer has developed against the antigen of interest, regular boosts and bleeds are performed to collect serum.

### Production of Mouse Monoclonal Antibodies:

Standard methods were employed for the generation of monoclonal antibodies using established hybridoma technology. This technology typically combines two steps, including step 1, in which an appropriate immunization is conducted (described above), and step 2, in which the fusion of B lymphocytes with immortal myeloma cells is carried out to generate hybrids capable of producing antibody molecules that exhibit immortality in cell culture. The generated hybridoma cells are re-cloned and diluted to obtain stable monoclonal cell cultures secreting the desired monoclonal antibody in the culture supernatant. The supernatants are tested e.g., in enzyme-linked immunosorbent assays (ELISA) for antigen specificity against MR-proADM. After the selection of appropriate cell clones, cells are transferred to large-scale cultivation to produce the desired antibody molecule in larger amounts. The purification of the antibodies is routinely performed by affinity chromatography.

### Determination of Antibody Sequence:

Antibody sequences were determined using next generation sequencing. Hybridomas were provided to the sequencing provider, total RNA was extracted and a cDNA library was created using RT-PCR. The cDNA library was subsequently sequenced using next generation sequencing (Illumina HiSeq sequencer). Contigs were then joined and antibody sequences were identified. Sequences of known aberrant (non-functional) antibody genes, which frequently occur in hybridoma cell lines, were not included in the analysis.

Alternatively, antibody sequences were determined using peptide mapping via liquid-chromatography coupled with mass spectrometry (LC/MS). De novo protein sequencing is a well-established procedure that relies on mass spectrometry to sequence proteins. This technique is advantageous as it does not require information on the amino acid sequence of peptides to be already known, circumventing limitations of methods that rely on protein sequence databases. De novo protein sequencing produced results directly from the acquired mass spectrometry data allowing to identify the CDR sequences. LC-MS analysis was conducted according to established protocols. The identified sequences were used to perform a classical bottom-up search against DDA data with FragPipe software.

Based on next generation (nucleic acid) and de novo (protein) sequencing, antibody sequences were assessed using three methods of CDR and variable domain annotation, according to standard techniques, for example the Protein Metrics software or the Geneious antibody annotation software. Antibodies have a predictable secondary structure. Standard schemes are available for annotating the CDR regions of a linear protein sequence, the most common of which are referred to as Chothia (Al-Lazikani, B., et al (1997). Journal of Molecular Biology, 273(4), 927-948) or Kabat (Kabat E A, et al, 1991, U.S. Department of Health and Human Services, Public Health Service, National Institutes of Health), which are widely adopted sequence-based schemes for numbering antibody residues. In preferred embodiments, the Kabat CDR annotation is employed, for example, as in SEQ ID NO 134-203.

In embodiments, any given CDR sequence presented herein may be combined with any other CDR sequence. In embodiments, CDR sequences from the same annotation method may be combined with each other, for example to form a combination of three L-CDRs or three H-CDRs. In embodiments, CDR sequences from different annotation methods but from the same VH or VL sequence may be combined with each other, for example to form a combination of three L-CDRs or three H-CDRs. In embodiments, CDR annotations using any one or more of methods 1, 2 or 3 from the same VH or VL sequence may be combined with each other, for example to form a combination of three L-CDRs or three H-CDRs.

The antibody sequences obtained are presented in the sequence table above.

### Antibody Stability:

Nano-DSF measurements were employed to determine the 7 antibody's stability and aggregation. No aggregation was observed under the following conditions:

| | IgG | | | | | | | |
|---|---|---|---|---|---|---|---|---|
| Ab | class | Origin | pH | Conc. | | Volume | Buffer | T ° C. |
| 1 | IgG2b | mouse | 8.5 | 2.95 | mg/mL | 100 μL | PBS, pH 7.4 (no | 2-8° C. |
| | k | | | 19.6 | μM | | preservatives) | |
| 2 | IgG1 k | mouse | 8.5 | 3.45 | mg/mL | 100 μL | PBS, pH 7.4 (no | 2-8° C. |
| | | | | 23 | μM | | preservatives) | |
| 3 | IgG2b | mouse | 8.0-8.5 | 1.20 | mg/mL | 100 μL | PBS, pH 7.4 (no | 2-8° C. |
| | k | | | 8 | μM | | preservatives) | |
| 4 | IgG2a | mouse | 7.8-8.3 | 1.30 | mg/mL | 100 μL | PBS, pH 7.4 (no | 2-8° C. |
| | k | | | 8.6 | μM | | preservatives) | |
| 5 | IgG1 k | mouse | 8.0-8.3 | 2.65 | mg/mL | 100 μL | PBS, pH 7.4 (no | 2-8° C. |
| | | | | 17.6 | μM | | preservatives) | |
| 6 | IgG1 k | mouse | 7.8-8.0 | 1.30 | mg/mL | 100 μL | PBS, pH 7.4 (no | 2-8° C. |
| | | | | 8.6 | μM | | preservatives) | |
| 7 | IgG2a | mouse | 7.8-8.3 | 1.85 | mg/mL | 100 μL | PBS, pH 7.4 (no | 2-8° C. |
| | k | | | 12.3 | μM | | preservatives) | |

Additionally, thermo-stability of proteins was measured on the Prometheus NT.48 apparatus by monitoring changes in auto fluorescence (from tyrosine or phenylalanine residues) on temperature gradient. For determination of the protein melting point Tm where half of the protein is unfolded, the ratio of the fluorescence intensities (F350/F330) can be plotted against temperature.

The protein samples were subjected to a linear thermal ramp (2 C/min, from 20 C to 90° C.), and fluorescence at 350 nm and 330 nm was collected at a rate of 10 data points per minute. Unfolding transition midpoints were determined automatically from the first derivative of the fluorescence ratio (F350/F330).

Antibodies concentration: 200 μg/mL, Buffer: PBS, Excitation Power: 90%, Temperature Slope 2.0° C./min. Aggregation analysis was simultaneous measured by diffusion of light on the same temperature ramp. The temperature of the onset of scattering corresponds to the beginning of aggregation.

| | | | Inflection | Inflection | |
|---|---|---|---|---|---|
| | | | Point #1 for | Point #2 for | |
| Sample | Sub | Onset #1 | Ratio (Tm1) | Ratio (Tm2) | Onset #1 for |
| ID | classes | for Ratio | CH2 domain | Fab frag. | Scattering |
| Ab1 | IgG2b k | 58.5° C. | 64.2° C. | 73.7° C. | 66.8° C. |
| Ab2 | IgG1 k | | 65.1° C. | 71.8° C. | |
| Ab3 | IgG2b k | 67.9° C. | | 74.0° C. | |
| Ab4 | IgG2a k | 62.0° C. | 69.2° C. | | 65.5° C. |
| Ab5 | IgG1 k | 64.7° C. | | 71.1° C. | 72.5° C. |
| Ab6 | IgG1 k | 61.1° C. | 63.9° C. | 70.2° C. | 77.9° C. |
| Ab7 | IgG2a k | 54.5° C. | 58.8° C. | 77.1° C. | 76.0° C. |

Ab 7 appears relatively unstable, although not below an acceptable level (Tm1=58.8° C. compared to 63-69° C. for the other Abs). Scattering (light diffusion) aggregation shows an aggregation tendency according to: Ac4>Ac1>Ac5>Ac7>Ac6. Surprisingly, no aggregation was detected for Ac2 and Ac3. For Ac1, Ac4 and Ac5, the aggregation Temperature starts before the Temperature of first Tm. All antibody showed sufficient stability and aggregation properties for the intended assay.

The aggregation and Tm studies were repeated under differing conditions. Thermal unfolding of antibodies and aggregation by NanoDSF was assessed, with antibody concentration: 1 mg/mL, Buffer: PBS, Excitation Power: 40%, Temperature Slope: 2.0° C./min, Capillar high sensitivity. Thermo-stability of proteins was measured on the Prometheus NT.48 apparatus by monitoring changes in auto fluorescence (from tyrosine or phenylalanine residues) on temperature gradient. For determination of the protein melting point Tm where half of the protein is unfolded, the ratio of the fluorescence intensities (F350/F330) can be plotted against temperature.

The protein samples were subjected to a linear thermal ramp (2° C./min, from 20° C. to 90° C.), and fluorescence at 350 nm and 330 nm was collected at a rate of 10 data points per minute. Unfolding transition midpoints were determined automatically from the first derivative of the fluorescence ratio (F350/F330). Aggregation analysis was simultaneous measured by diffusion of light on the same temperature ramp. The temperature of the onset of scattering corresponds to the beginning of aggregation.

| | | | Tm1 | | | |
|---|---|---|---|---|---|---|
| | Sub | Onset | CH2 | Tm2 | Tm3 | |
| Sample ID | classes | Tm | domain | Fab frag. | CH3 domain | Onset Tagg |
| Ab2_lot a | IgG1 k | 64.8° C. | 64.9° C. | 72.1° C. | 80.6° C. | 67.5° C. |
| Ab2_lot b | IgG1 k | 64.7° C. | 64.8° C. | 72.0° C. | 79.7° C. | 65.1° C. |
| Ab2_lot c | IgG1 k | 64.3° C. | 64.9° C. | 72.0° C. | 79.9° C. | 66.7° C. |
| Ab3_lot a | IgG2b k | 68.5° C. | 74.1° C. | | | 65.4° C. |
| Ab3_lot b | IgG2b k | 70.2° C. | 74.3° C. | | | 67.4° C. |
| Ab3_lot c | IgG2b k | 70.6° C. | 74.7° C. | | | 67.0° C. |
| Ab3_lot d | IgG2b k | 70.2° C. | 74.2° C. | | | 62.9° C. |
| Ab4 | IgG2a k | 61.0° C. | 70.6° C. | | 78.4° C. | 66.9° C. |
| Ab5 | IgG1 k | 67.6° C. | | 73.2° C. | 79.8° C. | 68.2° C. |

Assessment of melting point (fluorescence) and stability concurred with earlier experiments. On a positive note, the different batches of Ab2 and Ab3 gave the same profiles, showing a reliable set of characteristics. Under these conditions Ab5 appears stable. All antibody showed sufficient stability and aggregation properties for the intended assay.

### Affinity Measurements of the Identified Antibodies:

Surface Plasmon Resonance was employed to determine the antibody affinity of Ab2, Ab3 and Ab5, by way of example, to the MR-proADM target. Two separate preparations of MR-proADM target were employed to test affinity. The test antigens were synthetic peptides comprising the entire MR-proADM sequence as set out in SEQ ID NO 3. The peptides were maintained at 1 mg/mL (195.5 μM), phosphate buffer, BSA free protease 0.1%, EDTA 6 mmol/L, and pH 7.0, and had a mass of 5113.8 g/moL.

Ab2, Ab3 and Ab5 interact with Antigens 1 and 2. The affinities are presented below:

Antigen 1:

| | | | | | Rmax | | | |
|---|---|---|---|---|---|---|---|---|
| Ab | ka (1/Ms) | SE(ka) | kd (1/s) | SE(kd) | (RU) | SE(Rmax) | KD (M) | Chi² (RU²) |
| 2 | 1.94E+06 | 1.80E+04 | 0.004816 | 3.20E−05 | 42.8 | 0.039 | 2.49E−09 | 0.382 |
| 3 | 5.21E+05 | 2.40E+03 | 0.007246 | 2.10E−05 | 30.7 | 0.019 | 1.39E−08 | 0.0687 |
| 5 | 6.58E+05 | 6.10E+03 | 0.004257 | 2.90E−05 | 6.3 | 0.041 | 6.47E−09 | 4.64 |

SPR binding data is shown in FIG. 1.

Antigen 2:

| | | | | | | Rmax | | | Chi² |
|---|---|---|---|---|---|---|---|---|---|
| Ab | Curve | ka (1/Ms) | SE(ka) | kd (1/s) | SE(kd) | (RU) | SE(Rmax) | KD (M) | (RU²) |
| 2 | Cycle: 7 | 2.33E+06 | 9.90E+03 | 0.006003 | 2.00E−05 | 39.6 | 0.016 | 2.57E−09 | 5.44E−02 |
| 3 | Cycle: 9 | 4.75E+05 | 1.20E+03 | 0.008274 | 1.40E−05 | 30.7 | 0.011 | 1.74E−08 | 0.0191 |
| 5 | Cycle: 13 | 5.01E+05 | 2.00E+04 | 0.05076 | 0.0016 | 33.5 | 0.3 | 1.01E−07 | 2.13E−01 |

SPR binding data is shown in FIG. 2. The antibodies show reproducible results over multiple trials and using multiple preparations of antigen. Additional experiments with the remaining antibodies are ongoing.

Additional studies were repeated to determine affinity of the inventive antibodies. Surface Plasmon Resonance was again employed to determine the antibody affinity of Ab2, Ab3 and Ab5, by way of example, to the MR-proADM target. The test antigen was a synthetic peptide comprising MR-proADM (amino acids 45-92 of pre-proADM) as set out in SEQ ID NO 3.

SPR analyses were performed on a T200 apparatus at 25° C. in PBS-pH7.4 containing 0.05% P20 surfactant (Cytiva). For binding measurements, Ac were captured (1000-2000RU) on mouse anti-Fc immobilized on CM5S-dextran sensorship. Anti-Fc diluted in acetate buffer, pH 5, was covalently immobilized (10000-13000 RU) to a CM5S sensor chip using EDC/NHS activation, following the manufacturer's instructions (Kit mouse anti-Fc Cytiva). 100 nM of antigenic peptide were injected (injection time=120 s) at 50 μL/min. After a dissociation step of 400 s in running buffer, sensor surface was regenerated using 30 μL of Gly-HCl pH 1.7. The K_{D} values were calculated using a Langmuir 1:1 fitting model (BiaEvaluation3.2, Cytiva. All sensorgrams were corrected by subtracting the low signal from the control reference surface (without any immobilized protein) and blank buffer injections before fitting evaluation.

| | | | | | | SE(ka) | SE(kd) |
|---|---|---|---|---|---|---|---|
| Ligand | ka (1/Ms) | kd (1/s) | KD (M) | Rmax (RU) | Chi² (RU²) | (1/Ms) | (1/s) |
| Ab 2 - a | 1.21E+06 | 3.59E−03 | 2.97E−09 | 62.2 | 0.0784 | 3.43E+03 | 6.81E−06 |
| Ab 2 - b | 1.22E+06 | 3.68E−03 | 3.02E−09 | 64.5 | 0.0673 | 3.11E+03 | 6.38E−06 |
| Ab 2 - c | 1.29E+06 | 3.63E−03 | 2.82E−09 | 62.7 | 0.0513 | 3.05E+03 | 5.86E−06 |
| Ab 3 - a | 4.78E+05 | 7.05E−03 | 1.48E−08 | 43.9 | 0.212 | 3.03E+03 | 3.10E−05 |
| Ab 3 - b | 4.43E+05 | 7.56E−03 | 1.71E−08 | 42 | 0.11 | 2.14E+03 | 2.49E−05 |
| Ab 3 - c | 3.98E+05 | 7.48E−03 | 1.88E−08 | 43.3 | 0.107 | 1.91E+03 | 2.36E−05 |
| Ab 3 - d | 3.86E+05 | 7.75E−03 | 2.01E−08 | 45.8 | 0.0648 | 1.38E+03 | 1.82E−05 |
| Ab 5 | 2.91E+05 | 7.18E−02 | 2.47E−07 | 56.3 | 0.393 | 1.07E+04 | 5.90E−04 |

### Testing Functional Properties of the Identified Antibodies:

The inventive antibodies were employed in an immunoluminometric assay to determine function and make comparisons to established methods such as the B⋅R⋅A⋅H⋅M⋅S MR-proADM KRYPTOR™ assay, based on polyclonal antibodies against MR-proADM. As can be seen from FIGS. 3-5, the inventive antibodies show improvements in signal over the B⋅R⋅A⋅H⋅M⋅S MR-proADM KRYPTOR™ assay when analysing plasma samples with fixed amounts of the MR-proADM target. Samples were provided from healthy patients (low MR-proADM), quality control samples were run in parallel, and samples with 4 nmol/L MR-proADM, either in EDTA or Heparin-treated plasma samples, were analysed using different combinations of the inventive antibodies. Antibodies were labelled appropriately (Tb=Terbium donor. AF=Alexa Fluorophore acceptor) and analysed in in an immunoluminometric assay system.

Experimental conditions were as follows:
12 mAbs, approx. 140 tested pairs on Pherastar.
Conjugate concentrations: L4: 0.3 μg/mL—AF: 3 μg/mL.
Buffer conditions: TRIS 0.1 M, pH7, Mouse IgG 0.1 mg/mL, BSA 0.1%, IgG bovines 0.5 mg/mL.
7 samples/condition (EDTA, HP, QC)—T0, 15, 30, 60 min.
Sample volume: 26 μL on B⋅R⋅A⋅H⋅M⋅S MR-proADM KRYPTOR, 50 μL for the new (inventive) MR-proADM 2.0 mAb couples.

Measurements were made by determining a “unitless” ratio fluorescent unit (RFU), in essence a measurement of fluorescent signal emitted from the fluorophores employed in the assay.

As can be derived from FIGS. 3-5, the following antibody combination achieved detection signals:
Ab1 with Ab2 or Ab3,
Ab2 with Ab1, Ab4, Ab3, Ab5 or Ab6,
Ab4 with Ab2,
Ab3 with Ab1, Ab2 or Ab7,
Ab5 with Ab2,
Ab6 with Ab2, and
Ab7 with Ab3.

Especially beneficial combinations were observed employing Ab1, Ab2, Ab3, Ab4 and Ab5, in varying combinations. Improvements were observed in RFU measurement over the B⋅R⋅A⋅H⋅M⋅S MR-proADM KRYPTOR™ assay control.

FIG. 5 shows an excerpt of the complete data emphasizing the beneficial results achieved using the combination of Ab2 and Ab3.

### Optimization of Antibody Pairing:

The experiments described above were repeated using the most promising antibody pairs and in different combinations of donor and acceptor fluorophores, using EDTA and Heparin plasma samples at different proADM concentrations.

The conditions employed were as follows:
KRYPTOR Gold KG0198+Comparative method B⋅R⋅A⋅H⋅M⋅S MR-proADM KRYPTOR,
Conjugate concentrations: Terbium (Tb): 0.3 μg/mL—AF: 3 μg/mL
Buffer conditions: TRIS 0.1 M, pH7, Mouse IgG 0.1 mg/mL, BSA 0.1%, IgG bovines 0.5 mg/mL
Sample volume: 26 μL on B⋅R⋅A⋅H⋅M⋅S MR-proADM KRYPTOR, 50 μL for the new (inventive) assays “MR-proADM 2.0 mAb” antibody pairs.
59 min analysis time.

| | B•R•A•H•M•S | | | | | | | | |
|---|---|---|---|---|---|---|---|---|---|
| | MR-proADM | Ab1-Tb/ | Ab2-Tb/ | Ab2-Tb/ | Ab2-Tb/ | Ab3-Tb/ | Ab5-Tb/ | Ab4-Tb/ | Ab2-Tb/ |
| Samples* | KRYPTOR | Ab2-AF | Ab1_AF | Ab3-AF | Ab5-AF | Ab2-AF | Ab2-AF | Ab2-AF | Ab4-AF |
| EDTA Plasma - | 0.91 | 0.28 | 0.32 | 8.91 | 0.08 | 3.69 | 0.18 | 0.20 | 0.65 |
| 1.00 nmol/L | | | | | | | | | |
| Heparin | 0.97 | 0.43 | 0.42 | 10.7 | −1.9 | 4.30 | 0.24 | 0.19 | 0.70 |
| Plasma - 1.04 | | | | | | | | | |
| nmol/L | | | | | | | | | |
| EDTA Plasma - | 0.10 | 0.00 | −0.01 | 0.54 | −4.0 | 0.23 | −0.02 | −0.01 | 1.61 |
| 0.17 nmol/L | | | | | | | | | |
| Heparin | 0.17 | 0.04 | 0.02 | 1.07 | 0.00 | 0.38 | 0.00 | −0.01 | 0.10 |
| Plasma - 0.26 | | | | | | | | | |
| nmol/L | | | | | | | | | |

As can be observed from the results, the combination of monoclonal antibodies Ab2 and Ab3, in both configurations with donor and acceptor fluorophores, produced excellent results, indicating superior detection compared to the established MR-proADM assay based on polyclonal antibodies. Signal strength was surprisingly enhanced over the established method by up to approximately 10-fold.

### Buffer Variation:

The experiments described above were repeated using the most promising antibody pairs, in different combinations of donor and acceptor fluorophores, using EDTA and Heparin plasma samples at different proADM concentrations, and using different buffer components, specifically both TRIS and Phosphate buffers were assessed.

The conditions employed were as follows:
2 systems+comparative method B⋅R⋅A⋅H⋅M⋅S MR-proADM KRYPTOR,
Terbium (Tb): 0.3 μg/mL—AF: 3 μg/mL,
TRIS 0.1M, pH7, Mouse IgG 0.1 mg/mL, BSA 0.1%, IgG bovines 0.5 mg/mL (freshly prepared)
Phosphate 0.1 M, pH7, Mouse IgG 0.1 mg/mL, BSA 0.1%, IgG bovines 0.5 mg/mL (freshly prepared)
Sample volume: 26 μL on B⋅R⋅A⋅H⋅M⋅S MR-proADM KRYPTOR, 50 μL for the new (inventive) assays “MR-proADM 2.0 mAb” antibody pairs.
29 min analysis time.

| | B•R•A•H•M•S MR- | | | | | |
|---|---|---|---|---|---|---|
| | proADM KRYPTOR | | | | | |
| | Conc. | | Ac3-Tb/Ac2-AF | | Ac2-Tb/Ac3-AF | |
| Sample Id | (nmol/L) | RFU | TRIS | PHOSPHATE | TRIS | PHOSPHATE |
| EDTA Plasma - | 0.17 | 0.107 | 0.203 | 0.228 | 0.527 | 0.519 |
| 0.17 nmol/L | | | | | | |
| EDTA Plasma - | 1.00 | 0.833 | 2.539 | 2.941 | 6.092 | 6.752 |
| 1.00 nmol/L | | | | | | |
| Heparin Plasma - | 0.26 | 0.159 | 0.324 | 0.367 | 0.893 | 0.943 |
| 0.26 nmol/L | | | | | | |
| Heparin Plasma - | 1.04 | 0.765 | 2.869 | 3.269 | 7.305 | 7.717 |
| 1.04 nmol/L | | | | | | |
| B•R•A•H•M•S, | 4.1 | 3.913 | 59.217 | 112.134 | 111.542 | 182.001 |
| MR-proADM | | | | | | |
| KRYPTOR ™ | | | | | | |
| QC2020 | | | | | | |

As can be observed from the results, the combination of monoclonal antibodies Ab2 and Ab3, in both configurations with donor and acceptor fluorophores, produced excellent results, both in Tris and phosphate buffers.

### Fluorophore Variation:

Further analysis was carried out to assess differences in performance when using various fluorophores. The experiments described above were repeated using either Europium cryptate (“−K”) or Terbium cryptate (“−Tb”) donor fluorophores, attached to Ab2, and comparisons were made to the existing product, in both EDTA and heparin plasma samples.

The conditions employed were as follows:
Europium Cryptate/AF647 (PO4+KF600 mM buffer) vs Terbium/AF (PO4 buffer) vs B⋅R⋅A⋅H⋅M⋅S MR-proADM KRYPTOR.
MR-proADM standards, EDTA & heparin plasma pools
Sample volume: 26 μL on B⋅R⋅A⋅H⋅M⋅S MR-proADM KRYPTOR, 50 μL for the new (inventive) assays “MR-proADM 2.0 mAb” antibody pairs.
29 min analysis time.

| | | | Ab2-K | Ab2-Tb |
|---|---|---|---|---|
| | B•R•A•H•M•S | | 0.45 μg/mL/ | 0.3 μg/mL/ |
| | MR-proADM | B•R•A•H•M•S | Ab3-AF647 | Ab3-AF |
| | KRYPTOR ™ | MR-proADM | 4 μg/mL | 3 μg/mL |
| | Ref. Conc. | KRYPTOR ™ | Europium | Terbium |
| Sample | (nmol/L) | RFU | RFU | RFU |
| EDTA Plasma Sample 1 | 0.12 | 0.09 | 0.05 | 0.47 |
| EDTA Plasma Sample 2 | 1.01 | 0.77 | 0.73 | 6.39 |
| B•R•A•H•M•S MR- | 3.80 | 3.93 | 28.4 | 181 |
| proADM KRYPTOR ™ | | | | |
| QC level 2 lot 020 | | | | |
| Heparin Plasma - | 0.23 | 0.16 | 0.09 | 0.89 |
| Sample 1 | | | | |
| Heparin Plasma - | 0.91 | 0.87 | 0.84 | 7.77 |
| Sample2 | | | | |

As can be observed from the results, the Europium-labelled Ab2 lead to excellent results, showing improvements over the QC sample measured using both the novel and established assays, whereby the Terbium-labelled Ab2 lead to further enhancements in signal, over both the established assay and the Europium assay using the novel monoclonal antibodies. As can be observed from the results, The Europium-labelled Ab2 has the similar level of signal on plasma samples compared to the established assay with polyclonal antibody, while the Terbium-labelled Ab2 leads to further enhancements in signal on plasma patient samples compared to the Europium-labelled Ab2.

Additional Comparisons with Established Assay:

Method comparisons were conducted between the established B⋅R⋅A⋅H⋅M⋅S MR-proADM assay and the new (inventive) assay “MR-proADM 2.0 mAb”, using the Ac2-Tb/Ab3-AF assay format, in two separate comparisons, namely using 57 EDTA plasma samples (KG0007; FIG. 6) and 29 EDTA plasma samples (KG0166; FIG. 7). Quantitative MR-proADM measurements were determined for individual samples to compare performance of the two assays. The results reveal excellent agreement between the two assays when calculating MR-proADM levels in EDTA plasma samples. Spearmans rs values of 0.993 and 0.987, respectively, were achieved in the two comparisons, with Fisher Exact test values of 0.989-0.996 and 0.972-0.994, respectively.

1. An in vitro method for determining mid-regional pro-adrenomedullin (MR-proADM) in a sample, comprising:
contacting the sample with
i. a first monoclonal antibody or fragment thereof, specific for a first epitope of MR-proADM, and
ii. a second monoclonal antibody or fragment thereof, specific for a second epitope of MR-proADM; and
detecting the binding of the first and second monoclonal antibodies or fragments thereof to said MR-proADM,
wherein the first and second antibodies are labeled with detectable labels, wherein a signal generated by said labels is modulated when the first and second antibodies are bound in an antibody-MR-proADM complex.

2. The method according to claim 1, wherein the first antibody and second antibody are dispersed in a liquid reaction mixture.

3. The method according to claim 1, wherein the first antibody is labelled with a donor label and the second antibody is labelled with an acceptor label, wherein proximity of donor and acceptor labels in the antibody-MR-proADM complex and a spectral overlap between a donor emission and acceptor absorption spectra intensifies the signal and/or extends the life span of the signal allowing for the measurement of temporally delayed fluorescence.

4. (canceled)

5. The method according to claim 1, wherein the donor label comprises a rare earth cryptate or chelate, and the acceptor label comprises a fluorescent or chemiluminescent dye.

6. The method according to claim 1, wherein the donor label comprises a terbium cryptate or chelate and the acceptor label comprises a fluorescent dye with excitation and emission spectra compatible with the terbium excitation and emission spectra.

7. The method according to claim 1, wherein the donor label comprises a terbium cryptate or chelate with emission spectra at 485-495, 540-550, 585-595 and/or 615-625 nm, and the acceptor label comprises a fluorescent dye with a excitation spectrum corresponding to the donor label emission spectrum, and an emission spectrum distinct from the donor label emission spectra.

8. (canceled)

9. The method according to claim 1, wherein the first and second epitopes are different from each other and selected from MR-proADM epitopes according to SEQ ID NO 5-18.

10. The method according to claim 1, wherein the first and/or second antibody comprises VH and VL domains that comprise the sequences according to SEQ ID NO 136 and 137 (Ab1), 151 and 152 (Ab2), 166 and 167 (Ab3), 181 and 182 (Ab4), 196 and 197 (Ab5), 211 and 212 (Ab6), or 226 and 227 (Ab7), respectively.

11. The method according to claim 1, wherein the first and second epitopes are different and selected from SEQ ID NO 7 or 8 (epitope of Ab2), and SEQ ID NO 9 or 10 (epitope of Ab3), and wherein the donor label comprises a terbium cryptate or chelate, and the acceptor label comprises a fluorescent dye with excitation and emission spectra compatible with the terbium excitation and emission spectra.

12. The method according to claim 1, wherein the first and second antibodies are different, and each comprise 6 CDR sequences, according to:
H-CDR1 according to SEQ ID NO 153 or 154, H-CDR2 according to SEQ ID NO 155 or 156, H-CDR3 according to SEQ ID NO 157 or 158, L-CDR1 according to SEQ ID NO 159 or 160, L-CDR2 according to SEQ ID NO 161 or 162, and L-CDR3 according to SEQ ID NO 163 (Ab2), and
H-CDR1 according to SEQ ID NO 168 or 169, H-CDR2 according to SEQ ID NO 170 or 171, H-CDR3 according to SEQ ID NO 172 or 173, L-CDR1 according to SEQ ID NO 174 or 175, L-CDR2 according to SEQ ID NO 176 or 177, and L-CDR3 according to SEQ ID NO 178 (Ab3),
and wherein the donor label comprises a terbium cryptate or chelate, and the acceptor label comprises a fluorescent dye with excitation and emission spectra compatible with the terbium excitation and emission spectra.

13. The method according to claim 10, wherein the first and second antibodies are different and comprise VH and VL domains according to SEQ ID NO 151 and 152 (Ab2), and SEQ ID NO 166 and 167 (Ab3).

14. The method according to claim 1, wherein the first and second epitopes are different and selected from SEQ ID NO 7 or 8 (epitope of Ab2), and SEQ ID NO 11 or 12 (epitope of Ab4), and wherein the donor label comprises a terbium cryptate or chelate and the acceptor label comprises a fluorescent dye with excitation and emission spectra compatible with the terbium excitation and emission spectra.

15. The method according to claim 1, wherein the first and second antibodies are different, and each comprise 6 CDR sequences, according to:
H-CDR1 according to SEQ ID NO 153 or 154, H-CDR2 according to SEQ ID NO 155 or 156, H-CDR3 according to SEQ ID NO 157 or 158, L-CDR1 according to SEQ ID NO 159 or 160, L-CDR2 according to SEQ ID NO 161 or 162, and L-CDR3 according to SEQ ID NO 163 (Ab2), and
H-CDR1 according to SEQ ID NO 183 or 184, H-CDR2 according to SEQ ID NO 185 or 186, H-CDR3 according to SEQ ID NO 187 or 188, L-CDR1 according to SEQ ID NO 189 or 190, L-CDR2 according to SEQ ID NO 191 or 192, and L-CDR3 according to SEQ ID NO 193 (Ab4),
and wherein the donor label comprises a terbium cryptate or chelate, and the acceptor label comprises a fluorescent dye with excitation and emission spectra compatible with the terbium excitation and emission spectra.

16. The method according to claim 13, wherein the first and second antibodies are different and comprise VH and VL domains according to SEQ ID NO 151 and 152 (Ab2), and SEQ ID NO 181 and 182 (Ab4).

17. The method according to claim 1, wherein the first and second epitopes are different and selected from SEQ ID NO 5 or 6 (epitope of Ab1), and SEQ ID NO 7 or 8 (epitope of Ab2), and wherein the donor label comprises a terbium cryptate or chelate and the acceptor label comprises a fluorescent dye with excitation and emission spectra compatible with the terbium excitation and emission spectra.

18. The method according to claim 1, wherein the first and second antibodies are different, and each comprise 6 CDR sequences, according to:
H-CDR1 according to SEQ ID NO 138 or 139, H-CDR2 according to SEQ ID NO 140 or 141, H-CDR3 according to SEQ ID NO 142 or 143, L-CDR1 according to SEQ ID NO 144 or 145, L-CDR2 according to SEQ ID NO 146 or 147, and L-CDR3 according to SEQ ID NO 148 (Ab1), and
H-CDR1 according to SEQ ID NO 153 or 154, H-CDR2 according to SEQ ID NO 155 or 156, H-CDR3 according to SEQ ID NO 157 or 158, L-CDR1 according to SEQ ID NO 159 or 160, L-CDR2 according to SEQ ID NO 161 or 162, and L-CDR3 according to SEQ ID NO 163 (Ab2),
and wherein the donor label comprises a terbium cryptate or chelate, and the acceptor label comprises a fluorescent dye with excitation and emission spectra compatible with the terbium excitation and emission spectra.

19. The method according to claim 16, wherein the first and second antibodies are different and comprise VH and VL domains according to SEQ ID NO 136 and 137 (Ab1), 151 and 152 (Ab2).

20. An analytical, detection and/or diagnostic kit for use in the method of claim 1, comprising:
a first monoclonal antibody and a second monoclonal antibody, or fragments thereof, that bind a first and second epitope of MR-proADM, respectively, and
one or more donor labels comprising a rare earth cryptate or chelate, and one or more acceptor labels comprising a fluorescent or chemiluminescent dye, wherein the excitation and emission spectra of the acceptor label are compatible with the donor label,
wherein said labels are in physical proximity to said antibodies and/or are conjugated to said antibodies in said kit.

21. A monoclonal antibody or antibody fragment that binds mid-regional pro-adrenomedullin (MR-proADM), wherein the antibody or antibody fragment comprises CDR sequences according to:
H-CDR1 according to SEQ ID NO 138 or 139, H-CDR2 according to SEQ ID NO 140 or 141, H-CDR3 according to SEQ ID NO 142 or 143, L-CDR1 according to SEQ ID NO 144 or 145, L-CDR2 according to SEQ ID NO 146 or 147, and L-CDR3 according to SEQ ID NO 148 (Ab1),
H-CDR1 according to SEQ ID NO 153 or 154, H-CDR2 according to SEQ ID NO 155 or 156, H-CDR3 according to SEQ ID NO 157 or 158, L-CDR1 according to SEQ ID NO 159 or 160, L-CDR2 according to SEQ ID NO 161 or 162, and L-CDR3 according to SEQ ID NO 163 (Ab2),
H-CDR1 according to SEQ ID NO 168 or 169, H-CDR2 according to SEQ ID NO 170 or 171, H-CDR3 according to SEQ ID NO 172 or 173, L-CDR1 according to SEQ ID NO 174 or 175, L-CDR2 according to SEQ ID NO 176 or 177, and L-CDR3 according to SEQ ID NO 178 (Ab3),
H-CDR1 according to SEQ ID NO 183 or 184, H-CDR2 according to SEQ ID NO 185 or 186, H-CDR3 according to SEQ ID NO 187 or 188, L-CDR1 according to SEQ ID NO 189 or 190, L-CDR2 according to SEQ ID NO 191 or 192, and L-CDR3 according to SEQ ID NO 193 (Ab4), or
H-CDR1 according to SEQ ID NO 198 or 199, H-CDR2 according to SEQ ID NO 200 or 201, H-CDR3 according to SEQ ID NO 202 or 203, L-CDR1 according to SEQ ID NO 204 or 205, L-CDR2 according to SEQ ID NO 206 or 207, and L-CDR3 according to SEQ ID NO 208 (Ab5).

22. The monoclonal antibody or antibody fragment according to claim 19, the monoclonal antibody or antibody fragment comprises VH and VL domains that comprise the sequences according to SEQ ID NO 136 and 137 (Ab1), 151 and 152 (Ab2), 166 and 167 (Ab3), 181 and 182 (Ab4), or 196 and 197 (Ab5), respectively.

23. The monoclonal antibody or antibody fragment according to claim 19, wherein the monoclonal antibody or antibody fragment comprises the CDR sequences according to Ab2.


SEQUENCE LISTING (293 sequences)

SEQ ID NO: 1 - protein, 185 aa, Homo sapiens
MKLVSVALMYLGSLAFLGADTARLDVASEFRKKWNKWALSRGKRELRMSSSYPTGLADVKAGPAQTLIRPQDMKGASRSPEDSSPDAARIRVKRYRQSMNNFQGLRSFGCRFGTCTVQKLAHQIYQFTDKDKDNVAPRSKISPQGYGRRRRRSLPEAGPGRTLVSSKPQAHGAPAPPSGSAPHFL

SEQ ID NO: 2 - protein, 164 aa, Homo sapiens
ARLDVASEFRKKWNKWALSRGKRELRMSSSYPTGLADVKAGPAQTLIRPQDMKGASRSPEDSSPDAARIRVKRYRQSMNNFQGLRSFGCRFGTCTVQKLAHQIYQFTDKDKDNVAPRSKISPQGYGRRRRRSLPEAGPGRTLVSSKPQAHGAPAPPSGSAPHFL

SEQ ID NO: 3 - protein, 48 aa, Homo sapiens
ELRMSSSYPTGLADVKAGPAQTLIRPQDMKGASRSPEDSSPDAARIRV

SEQ ID NO: 4 - protein, 52 aa, Homo sapiens
YRQSMNNFQGLRSFGCRFGTCTVQKLAHQIYQFTDKDKDNVAPRSKISPQGY

SEQ ID NO: 5 - protein, 14 aa, Homo sapiens
LADVKAGPAQTLIR

SEQ ID NO: 6 - protein, 16 aa, Homo sapiens
CLADVKAGPAQTLIRC

SEQ ID NO: 7 - protein, 14 aa, Homo sapiens
RPQDMKGASRSPED

SEQ ID NO: 8 - protein, 15 aa, Homo sapiens
CRPQDMKGASRSPED

SEQ ID NO: 9 - protein, 12 aa, Homo sapiens
SSPDAARIRVKR

SEQ ID NO: 10 - protein, 16 aa, Homo sapiens
KLHCSSPDAARIRVKR

SEQ ID NO: 11 - protein, 12 aa, Homo sapiens
PEDSSPDAARIR

SEQ ID NO: 12 - protein, 16 aa, Homo sapiens
KLHCPEDSSPDAARIR

SEQ ID NO: 13 - protein, 12 aa, Homo sapiens
SSPDAARIRVKR

SEQ ID NO: 14 - protein, 16 aa, Homo sapiens
KLHCSSPDAARIRVKR

SEQ ID NO: 15 - protein, 14 aa, Homo sapiens
LADVKAGPAQTLIR

SEQ ID NO: 16 - protein, 18 aa, Homo sapiens
LADVKAGPAQTLIRCKLH

SEQ ID NO: 17 - protein, 14 aa, Homo sapiens
LADVKAGPAQTLIR

SEQ ID NO: 18 - protein, 18 aa, Homo sapiens
LADVKAGPAQTLIRCKLH

SEQ ID NO: 19 - protein, 469 aa, Mus musculus
EVQLQQSGPELVKPGASVKMSTTTSGYTFTEYTLHWVKPSALFTSSLEWLGSLLPKNNSSPPRSGGKALLTVNKSSSTAYMELRSLTSDDSAVYYPMGAATTGDQGTSHHMRSPMHWFPNWGQGTLVTVSAAKTTPPSVYPLAPGPFGCSPDWAFNLKGYFPESVTVTWNSGSLSSSVHTFPALLQSGLYPESSPKSVGGMSDGFGASGSEEDATGDATQGSTRDKKLEPSGPISTINPCAASHECKMMAPNLEGGPSVFIFPPNIKDVLMISLTPKVWNKSMLEPMGDPQVDIEDALAGQKAVFNDYFSDFSGVYSAGLSRVVSTLPIQHQDWMSGKEFKCKVNNKDLPSPIERTISKIKGLVRAPQVYILPPPAEQLSRKRAAYDRHAYKAVLSEGQLQQSGPELVKPAGSVKDTAPVLDSDGSYFIYSKLNMKPHGGWEKTDSFSPHHTGLKNYYLKKTISRSPGK

SEQ ID NO: 20 - protein, 221 aa, Mus musculus
DVPYTQTPLSLPVSLGDQASISFNSSQSIVHSNGNTYLEWYLQKPGQSPKLLIYKVSNRFSGVPDRFSGSGSGTDFTLKISRVEAEDLGVYYTDQGSHVPWTFGGGTKLEIKRADAAPTVSIFPPSSEKYSQGNVSAVGVTYDGHTALTRDIDNVKWKIDGSERQNGVLNSWTDQDSKDSTYSMSSTLTLTKDEYERHNSYTPDGPPATSTSPIVKSTNEC

SEQ ID NO: 21 - protein, 131 aa, Mus musculus
EVQLQQSGPELVKPGASVKMSTTTSGYTFTEYTLHWVKPSALFTSSLEWLGSLLPKNNSSPPRSGGKALLTVNKSSSTAYMELRSLTSDDSAVYYPMGAATTGDQGTSHHMRSPMHWFPNWGQGTLVTVSA

SEQ ID NO: 22 - protein, 112 aa, Mus musculus
DVPYTQTPLSLPVSLGDQASISFNSSQSIVHSNGNTYLEWYLQKPGQSPKLLIYKVSNRFSGVPDRFSGSGSGTDFTLKISRVEAEDLGVYYTDQGSHVPWTFGGGTKLEIK

SEQ ID NO: 23 - protein, 10 aa, Mus musculus
GYTFTEYTLH

SEQ ID NO: 24 - protein, 12 aa, Mus musculus
SGYTFTEYTLHW

SEQ ID NO: 25 - protein, 8 aa, Mus musculus
GYTFTEYT

SEQ ID NO: 26 - protein, 15 aa, Mus musculus
SLLPKNNSSPPRSGG

SEQ ID NO: 27 - protein, 17 aa, Mus musculus
GSLLPKNNSSPPRSGGK

SEQ ID NO: 28 - protein, 6 aa, Mus musculus
LLPKNN

SEQ ID NO: 29 - protein, 19 aa, Mus musculus
TGDQGTSHHMRSPMHWFPN

SEQ ID NO: 30 - protein, 21 aa, Mus musculus
TTGDQGTSHHMRSPMHWFPNW

SEQ ID NO: 31 - protein, 24 aa, Mus musculus
MGAATTGDQGTSHHMRSPMHWFPN

SEQ ID NO: 32 - protein, 16 aa, Mus musculus
NSSQSIVHSNGNTYLE

SEQ ID NO: 33 - protein, 11 aa, Mus musculus
QSIVHSNGNTY

SEQ ID NO: 34 - protein, 7 aa, Mus musculus
KVSNRFS

SEQ ID NO: 35 - skipped

SEQ ID NO: 36 - protein, 9 aa, Mus musculus
DQGSHVPWT

SEQ ID NO: 37 - protein, 457 aa, Mus musculus
AQLQLVQSGPELRKPGETVKISCKASGYTFTDYSMHWVKQVPGKVLKWMGWINTETGEPTYVDDFKGRFAFSLETSASTAYLRLGGLLKNEDTATYFAPHQHQHHNFKDVLEGNEQFLGGAAKGTVSSAKTTPPSVYPLAPGSAAQTNSMVTLGCLVKGYFPEPVTVTWNSGSLSSGVHTFPAVLQSDLYTLSSSVTVPSSTWPPCFMYPNVAHPASSTKVDKKIVPRQEYMKGNCTVPEVSSVFIFPPKPKDVLTITLTKPVPGPWDVDISKDDPEVQFSWFVDDVEVHTAQTQPREEQFNSTFRSVSELPPYHQDWLNGKEFKCRVNSAAFPAPIEKTISKTKGRPKAPQVYTIPPPKEQMAKDKVGMSMMSLSTTGGNSGSPVFGGEPHHNGQPAENYKNTQPIMDTDGSYFVYSKLNVQKSNWEAGNTFTCSVLHEGLHNHHTEKSLSHSPGK

SEQ ID NO: 38 - protein, 215 aa, Mus musculus
DVVMTQTPLTLSVTYLEKGPAKCKSSQSLLDSDGQTYLHWLLQRPGQSPKRLIYLVSKLGSGVPDRFTGSGSGTDFTLKSIRVEAEDLGLYYSSGGEGAPDLLESQALTEKAVVHNSSTLGPPSSSRYSQGNVSAVGVTYDGHTAITRKWKIDGSERQNGVLNSWTDQDSKDSTYSMSSTLTLTKDEYTSPGFSVVATHKTSTSPIVKSFNRNEC

SEQ ID NO: 39 - protein, 128 aa, Mus musculus
AQLQLVQSGPELRKPGETVKISCKASGYTFTDYSMHWVKQVPGKVLKWMGWINTETGEPTYVDDFKGRFAFSLETSASTAYLRLGGLLKNEDTATYFAPHQHQHHNFKDVLEGNEQFLGGAAKGTVSS

SEQ ID NO: 40 - protein, 112 aa, Mus musculus
DVVMTQTPLTLSVTYLEKGPAKCKSSQSLLDSDGQTYLHWLLQRPGQSPKRLIYLVSKLGSGVPDRFTGSGSGTDFTLKSIRVEAEDLGLYYSSGGEGAPDLLESQALTEKA

SEQ ID NO: 41 - protein, 10 aa, Mus musculus
GYTFTDYSMH

SEQ ID NO: 42 - protein, 8 aa, Mus musculus
GYTFTDYS

SEQ ID NO: 43 - protein, 17 aa, Mus musculus
WINTETGEPTYVDDFKG

SEQ ID NO: 44 - protein, 8 aa, Mus musculus
INTETGEP

SEQ ID NO: 45 - protein, 17 aa, Mus musculus
QHQHHNFKDVLEGNEQF

SEQ ID NO: 46 - protein, 16 aa, Mus musculus
KSSQSLLDSDGQTYLH

SEQ ID NO: 47 - protein, 11 aa, Mus musculus
QSLLDSDGQTY

SEQ ID NO: 48 - protein, 7 aa, Mus musculus
LVSKLGS

SEQ ID NO: 49 - skipped

SEQ ID NO: 50 - protein, 9 aa, Mus musculus
SGGEGAPDL

SEQ ID NO: 51 - protein, 10 aa, Mus musculus
SSGGEGAPDL

SEQ ID NO: 52 - protein, 455 aa, Mus musculus
QEVQLQQSGPELVRPGASVKNQSYLNLPDKDQLQALEKSLEKGNGRLNPYNGATNYNQNFKDKATLTVDKSSSTAYMELHSLTSEDSAVYYPHDGTRSFTTPPEAYWGQGTLVTVSAAKTTPPSVYPLAPGCGDWYPVMNGLVKHETPESVTVTWNSGSLSSSVHTFPALLQSGLYTMSSSVTVPSSTWPSQTVTCMTVSVAHPASSTTVDKKLEPSGPLSTLNWIGRLNPYDGATNYNQPAAKDKLEGGPSVFIFPPNIKDVLMISPKLTVPEGVGLSVADLGISVTPPGKVFNDYFSDFSGVYSAGLDKVVSTLPIQHQDWMSGKYDLDFKVNNKDLPSPIERTISKIKGLVRAPQVYILPPPAEQLSRKDMTHNKSIEWIGRLNPYNQTNYNQNFKDTAPVLDSDGSYFIYSKLPGVNTSLTVKTDSFSPANRHEGLKNYYLKKTISRSPGK

SEQ ID NO: 53 - protein, 222 aa, Mus musculus
YPLDTQAAFSNPVTLGTSASISVHSSKSLLYSNGLTYLFWYLQRPGQSPQLILYQMSNLASGVPDRFSGSGSGTDFTLRISRVEAEDVGPDCAQNLELPWTFGGGTKLEIKRADAAPTVSIFPPSSEKYSQGNVSAVGVTYDGHTALTRKDINVKWKIDGSERQNGVLNSWTDQDSKDSTYSMSSTLTLTKDEYERHPDEDCEATHKTSTSPIVKSFNRNEC

SEQ ID NO: 54 - protein, 117 aa, Mus musculus
QEVQLQQSGPELVRPGASVKNQSYLNLPDKDQLQALEKSLEKGNGRLNPYNGATNYNQNFKDKATLTVDKSSSTAYMELHSLTSEDSAVYYPHDGTRSFTTPPEAYWGQGTLVTVSA

SEQ ID NO: 55 - protein, 111 aa, Mus musculus
YPLDTQAAFSNPVTLGTSASISVHSSKSLLYSNGLTYLFWYLQRPGQSPQLILYQMSNLASGVPDRFSGSGSGTDFTLRISRVEAEDVGPDCAQNLELPWTFGGGTKLEIK

SEQ ID NO: 56 - protein, 14 aa, Mus musculus
LNLPDKDQLQALEK

SEQ ID NO: 57 - protein, 12 aa, Mus musculus
NLPDKDQLQALE

SEQ ID NO: 58 - protein, 8 aa, Mus musculus
PDKDQLQA

SEQ ID NO: 59 - protein, 8 aa, Mus musculus
LNPYNGAT

SEQ ID NO: 60 - protein, 14 aa, Mus musculus
HDGTRSFTTPPEAY

SEQ ID NO: 61 - protein, 16 aa, Mus musculus
HSSKSLLYSNGLTYLF

SEQ ID NO: 62 - protein, 11 aa, Mus musculus
KSLLYSNGLTY

SEQ ID NO: 63 - protein, 7 aa, Mus musculus
QMSNLAS

SEQ ID NO: 64 - skipped

SEQ ID NO: 65 - protein, 9 aa, Mus musculus
AQNLELPWT

SEQ ID NO: 66 - protein, 445 aa, Mus musculus
EVQLQQSGPELVKPGASVRIPCKTYGSTFTEYTMHWVKQLGRKSLEWIGGLNPNSGNSRFSAGKFKDRATLTVDKSSTTAYMELRSLTSEDSAVTPHTMSQPTHHHNYGDYGYYAMDYWGQGTSVTVSSAKTTAPSVYPLAPVCDAMCNYGVLGCKGYFPEPVTLTWNSGSLSSGVHTFPAVLQSDLYTLSSSVTVTSSTWPSQSITCNVAHPASSTKVDQLTKYSGEGDLALVKFSPNEQKNHLGEGWPVRAVLPEIVTVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKYSQGNVSAVGVTYDGHTALTRNGKTELNYKNTEPVLDSDGSYFMYSKLRVEKKNWVERNSYSCSVVHEGLHNHHTTKSFSRTPGK

SEQ ID NO: 67 - protein, 216 aa, Mus musculus
DIKMTQSPSSMYAFLGERVTITCKASQDIHRYLSWFQQKPGKSPKTLIYRANSLVDGVPSRFSGSGSGQDYSLTISDTLLLELKRMKLQFAEFPLTFGAGTKLELKRADAAPTVSIFPPSSEQLTSGGASVVTMGSSPSSMYPKDINVKWKIDGSERQNGVLNSWTDQDSKDSTYSMSSTLTLTKDEYERHNSYTCEATHKTSTSPIVKSFNRNEC

SEQ ID NO: 68 - protein, 129 aa, Mus musculus
EVQLQQSGPELVKPGASVRIPCKTYGSTFTEYTMHWVKQLGRKSLEWIGGLNPNSGNSRFSAGKFKDRATLTVDKSSTTAYMELRSLTSEDSAVTPHTMSQPTHHHNYGDYGYYAMDYWGQGTSVTVSS

SEQ ID NO: 69 - protein, 106 aa, Mus musculus
DIKMTQSPSSMYAFLGERVTITCKASQDIHRYLSWFQQKPGKSPKTLIYRANSLVDGVPSRFSGSGSGQDYSLTISDTLLLELKRMKLQFAEFPLTFGAGTKLELK

SEQ ID NO: 70 - protein, 10 aa, Mus musculus
GSTFTEYTMH

SEQ ID NO: 71 - protein, 8 aa, Mus musculus
GSTFTEYT

SEQ ID NO: 72 - protein, 18 aa, Mus musculus
GLNPNSGNSRFSAGKFKD

SEQ ID NO: 73 - protein, 17 aa, Mus musculus
LNPNSGNSRFSAGKFKD

SEQ ID NO: 74 - protein, 29 aa, Mus musculus
LNPNSGNSRFSAGKFKDRATLTVDKSSTT

SEQ ID NO: 75 - protein, 16 aa, Mus musculus
THHHNYGDYGYYAMDY

SEQ ID NO: 76 - protein, 11 aa, Mus musculus
KASQDIHRYLS

SEQ ID NO: 77 - protein, 6 aa, Mus musculus
QDIHRY

SEQ ID NO: 78 - protein, 7 aa, Mus musculus
RANSLVD

SEQ ID NO: 79 - protein, 6 aa, Mus musculus
ANSLVD

SEQ ID NO: 80 - skipped

SEQ ID NO: 81 - protein, 8 aa, Mus musculus
QFAEFPLT

SEQ ID NO: 82 - protein, 451 aa, Mus musculus
EVQLQQSGPELVKPGASVKISCKASGYSFTDPGDGMHWVKFSSSLEWIGRINPYNQTKYNQKFNDKASLTVDKGWSGDCELLSGWGNTKVYMKSALEPPKSTTHHWKMNSDGDYWGQGTTLRVSSAKHKMSVYPLAPGSAAQTNSMVTLGCLVKGYFPEPVTVTWNSGSLSSGVHTFPAVLQSDLYTLSSSVTVPSSTWPESAFVCDTVSAPSAPASPVHTRDTLLLELKRPEVSSVFIFPPKPKDVLTITLTPKVTSCMVVVDISKDDPEVQFSWFVDDVEVHTAQTQPREEQFNSTFRSVSELPPYHQDWLNGKEFKCRVNSAAFPAPIEKTISKTKGRPKAPQVYTIPPPKESSSTAYEMLHSLTSEDSAVYPEPSESSEKWQWNGQPAENYKNTQPIMDTDGSYFVYSKLNVQKSNWEAGNTFTCSVLHEGLHNHHTEKSLSHSPGK

SEQ ID NO: 83 - protein, 213 aa, Mus musculus
DIVMTQAAFSNPVTLGTSASIAGAKEASKSLLYSNGITYLYWYLQKPGQSPQLLIYQMSNLASGVPDRFSSSGSGTDFTLRIPTVSSSYLNLAQNLELPWTFGGGTKLNFKLREAVVTVKYSQGNVSAVGVTYDGHTALTRDINVKWKIDGSERQNGVLNSWTDQDSKDSTYSMSSTLTLTKDEYERHNSYTCEATHKTSTSPIVKSFNRNEC

SEQ ID NO: 84 - protein, 125 aa, Mus musculus
EVQLQQSGPELVKPGASVKISCKASGYSFTDPGDGMHWVKFSSSLEWIGRINPYNQTKYNQKFNDKASLTVDKGWSGDCELLSGWGNTKVYMKSALEPPKSTTHHWKMNSDGDYWGQGTTLRVSS

SEQ ID NO: 85 - protein, 111 aa, Mus musculus
DIVMTQAAFSNPVTLGTSASIAGAKEASKSLLYSNGITYLYWYLQKPGQSPQLLIYQMSNLASGVPDRFSSSGSGTDFTLRIPTVSSSYLNLAQNLELPWTFGGGTKLNFK

SEQ ID NO: 86 - protein, 10 aa, Mus musculus
GYSFTDPGDG

SEQ ID NO: 87 - protein, 8 aa, Mus musculus
YSFTDPGD

SEQ ID NO: 88 - protein, 12 aa, Mus musculus
SGYSFTDPGDGM

SEQ ID NO: 89 - protein, 7 aa, Mus musculus
INPYNQT

SEQ ID NO: 90 - protein, 5 aa, Mus musculus
NPYNQ

SEQ ID NO: 91 - protein, 9 aa, Mus musculus
RINPYNQTK

SEQ ID NO: 92 - protein, 8 aa, Mus musculus
KMNSDGDY

SEQ ID NO: 93 - protein, 9 aa, Mus musculus
WKMNSDGDY

SEQ ID NO: 94 - protein, 16 aa, Mus musculus
EASKSLLYSNGITYLY

SEQ ID NO: 95 - protein, 11 aa, Mus musculus
KSLLYSNGITY

SEQ ID NO: 96 - protein, 7 aa, Mus musculus
QMSNLAS

SEQ ID NO: 97 - skipped

SEQ ID NO: 98 - protein, 9 aa, Mus musculus
AQNLELPWT

SEQ ID NO: 99 - protein, 8 aa, Mus musculus
AQNLELPW

SEQ ID NO: 100 - protein, 454 aa, Mus musculus
QIQLVQSGPDLKKPGETVKISCKASGYSFTDFSVHWVKQSPGMGIKWMGWINTETGEPTYADDFKGRFAFSLETSASTAYLQINYLKGGDDTATYFLHMHSTASNVAPETVLPKMRHNRYDDDYWGQGTTLTVSSAKTTPPSVYPLAPGSAAQTNSMVTLGPTRVKFYGPEPVTVTWNSGSLSSGVHTFPAVLQSDLYTLSSSVTVPSSTWPSEAFDSWSVAQVASHTPTNLDVLEGNEQFLNAAKELTVAGDPKSVRDTLLLELKRDFCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLLPRSGTLAQDIPGRTPQYRSNGRENYKNTQPPYDTDGSYFVYSKLNVQGSSRAGETYPEPEPTPAPVPNYLASLSHSPGK

SEQ ID NO: 101 - protein, 221 aa, Mus musculus
DIVMTQSPSSLSVSAGEKVTMSCKSSQSLLNSGNGAKNYLAWYQQKPGQPPKRLIYGASTRQSGVPDRFTGSGSGTDFTLTISSVQAEDLAVYYCQNDHSYPLTFGAGTKLELKRADAAPTVSIFPPSSEQLTKYSGEGDNQSYLNLPKDSAGLSYIDGSERQNGVLNSWTDQDSKDSTYSMSSTLTLTKDEYERHNSYTCEATHKTSTSPIVKSFNRNEC

SEQ ID NO: 102 - protein, 135 aa, Mus musculus
QIQLVQSGPDLKKPGETVKISCKASGYSFTDFSVHWVKQSPGMGIKWMGWINTETGEPTYADDFKGRFAFSLETSASTAYLQINYLKGGDDTATYFLHMHSTASNVAPETVLPKMRHNRYDDDYWGQGTTLTVSS

SEQ ID NO: 103 - protein, 114 aa, Mus musculus
DIVMTQSPSSLSVSAGEKVTMSCKSSQSLLNSGNGAKNYLAWYQQKPGQPPKRLIYGASTRQSGVPDRFTGSGSGTDFTLTISSVQAEDLAVYYCQNDHSYPLTFGAGTKLELK

SEQ ID NO: 104 - protein, 10 aa, Mus musculus
GYSFTDFSVH

SEQ ID NO: 105 - protein, 8 aa, Mus musculus
GYSFTDFS

SEQ ID NO: 106 - protein, 17 aa, Mus musculus
WINTETGEPTYADDFKG

SEQ ID NO: 107 - protein, 8 aa, Mus musculus
INTETGEP

SEQ ID NO: 108 - protein, 16 aa, Mus musculus
ETVLPKMRHNRYDDDY

SEQ ID NO: 109 - protein, 14 aa, Mus musculus
VLPKMRHNRYDDDY

SEQ ID NO: 110 - protein, 27 aa, Mus musculus
HMHSTASNVAPETVLPKMRHNRYDDDY

SEQ ID NO: 111 - protein, 18 aa, Mus musculus
KSSQSLLNSGNGAKNYLA

SEQ ID NO: 112 - protein, 13 aa, Mus musculus
QSLLNSGNGAKNY

SEQ ID NO: 113 - protein, 7 aa, Mus musculus
GASTRQS

SEQ ID NO: 114 - skipped

SEQ ID NO: 115 - protein, 9 aa, Mus musculus
QNDHSYPLT

SEQ ID NO: 116 - protein, 8 aa, Mus musculus
NDHSYPLT

SEQ ID NO: 117 - protein, 444 aa, Mus musculus
QIQLVQSGPDLKNPGETVKISCKASGYSFTDFSVHWVKQSPGLHLKAMMGWINTETGEPTYADDFKGRFAFSLETSASTAYLQINYLKNDDTATYFAKDFCREHMVTARNRYDDDYWGQGTALTVSSAKTTAPSVYPLAPVPDFQTSQATTTGWVKHETPEPVTLTWNSGSLSSGVHTFPAVLQSDLVMTQSPVTLEKYSQGNVSAVGVTYDGHTALTRKKIEPRGPTIKPCMMKNCQSPNLLGGPSVFIFPPKIKDYDDDYWGKDGGRLVKGPDVQISWFVNNVEVFNDYFSDFSGVYSAGLSRVVSALPIQHQDWMSGKEFKCKVNNKDLPAPIERTISKPKGSVRAPQVYVLPPPEEEMTKKQVEGETDIDVMTPHDQQKTELNYKNTEPVLDSDGSYFMYSKLRVEKKNWVERNSYSCSVVHEGLHNHHTTKSFSRTPGK

SEQ ID NO: 118 - protein, 218 aa, Mus musculus
DIVMTQSPSSLSVSAGEKVTMSCKSSQSLLNSGNQKNYLAWYQQKPGQPPKRLIYGASTRQSGVPDRFTGSGSGTDFTLTISSVQAEYDLAQSKCQNDHSYPLTFGAGTKLELKRADAAPTVSIFPPSSEQLTSSTAAFTSSLNNFYPKDINVKWKIDGSERQNGVLNSWTDQDSKDSTYSMSSTLTLTKDEYERHVYFATHKTSTSPIVKSFNRNEC

SEQ ID NO: 119 - protein, 127 aa, Mus musculus
QIQLVQSGPDLKNPGETVKISCKASGYSFTDFSVHWVKQSPGLHLKAMMGWINTETGEPTYADDFKGRFAFSLETSASTAYLQINYLKNDDTATYFAKDFCREHMVTARNRYDDDYWGQGTALTVSS

SEQ ID NO: 120 - protein, 114 aa, Mus musculus
DIVMTQSPSSLSVSAGEKVTMSCKSSQSLLNSGNQKNYLAWYQQKPGQPPKRLIYGASTRQSGVPDRFTGSGSGTDFTLTISSVQAEYDLAQSKCQNDHSYPLTFGAGTKLELK

SEQ ID NO: 121 - protein, 10 aa, Mus musculus
GYSFTDFSVH

SEQ ID NO: 122 - protein, 11 aa, Mus musculus
GYSFTDFSVHW

SEQ ID NO: 123 - protein, 8 aa, Mus musculus
GYSFTDFS

SEQ ID NO: 124 - protein, 17 aa, Mus musculus
WINTETGEPTYADDFKG

SEQ ID NO: 125 - protein, 18 aa, Mus musculus
WINTETGEPTYADDFKGR

SEQ ID NO: 126 - protein, 8 aa, Mus musculus
INTETGEP

SEQ ID NO: 127 - protein, 14 aa, Mus musculus
EHMVTARNRYDDDY

SEQ ID NO: 128 - protein, 19 aa, Mus musculus
KDFCREHMVTARNRYDDDY

SEQ ID NO: 129 - protein, 17 aa, Mus musculus
KSSQSLLNSGNQKNYLA

SEQ ID NO: 130 - protein, 12 aa, Mus musculus
QSLLNSGNQKNY

SEQ ID NO: 131 - protein, 7 aa, Mus musculus
GASTRQS

SEQ ID NO: 132 - skipped

SEQ ID NO: 133 - protein, 9 aa, Mus musculus
QNDHSYPLT

SEQ ID NO: 134 - protein, 475 aa, Mus musculus
MGWSWIFLFLLSGTAGVLSEGQLQQSGPELVKPGASVKISCKTSGYTFTEYTIHWVKQSHGKSLEWIGSIIPKNAFTRYNQKFKGKAILTVDKSSSTAYMELRSLTSDDSAVYYCARPGYDGHIWFPNWGQGTLVTVSAAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 135 - protein, 238 aa, Mus musculus
MKLPVRLLVLMFWIPASSSDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 136 - protein, 120 aa, Mus musculus
EGQLQQSGPELVKPGASVKISCKTSGYTFTEYTIHWVKQSHGKSLEWIGSIIPKNAFTRYNQKFKGKAILTVDKSSSTAYMELRSLTSDDSAVYYCARPGYDGHIWFPNWGQGTLVTVSA

SEQ ID NO: 137 - protein, 112 aa, Mus musculus
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPWTFGGGTKLEIK

SEQ ID NO: 138 - protein, 8 aa, Mus musculus
GYTFTEYT

SEQ ID NO: 139 - protein, 5 aa, Mus musculus
EYTIH

SEQ ID NO: 140 - protein, 8 aa, Mus musculus
IIPKNAFT

SEQ ID NO: 141 - protein, 17 aa, Mus musculus
SIIPKNAFTRYNQKFKG

SEQ ID NO: 142 - protein, 13 aa, Mus musculus
ARPGYDGHIWFPN

SEQ ID NO: 143 - protein, 11 aa, Mus musculus
PGYDGHIWFPN

SEQ ID NO: 144 - protein, 11 aa, Mus musculus
QSIVHSNGNTY

SEQ ID NO: 145 - protein, 16 aa, Mus musculus
RSSQSIVHSNGNTYLE

SEQ ID NO: 146 - skipped

SEQ ID NO: 147 - protein, 7 aa, Mus musculus
KVSNRFS

SEQ ID NO: 148 - protein, 9 aa, Mus musculus
FQGSHVPWT

SEQ ID NO: 149 - protein, 456 aa, Mus musculus
MAWVWTLLFLMAAAQSIQAQIQLVQSGPELRKPGETVKISCKASGYTFTDYSMHWVKQVPGKVLKWMGWINTETGEPTYVDDFKGRFAFSLETSASTAYLRINILKNEDAATYFCATNFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 150 - protein, 238 aa, Mus musculus
MSPAQFLFLLVLWIRETNGDVVMTQTPLTLSVSIGQPASISCKSSQSLLDSDGQTYLHWLLQRPGQSPKRLIYLVSKLGSGVPDRFTGSGSGTDFTLKISRVEAEDLGLYYCWQGTHFPRTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 151 - protein, 113 aa, Mus musculus
QIQLVQSGPELRKPGETVKISCKASGYTFTDYSMHWVKQVPGKVLKWMGWINTETGEPTYVDDFKGRFAFSLETSASTAYLRINILKNEDAATYFCATNFDYWGQGTTLTVSS

SEQ ID NO: 152 - protein, 112 aa, Mus musculus
DVVMTQTPLTLSVSIGQPASISCKSSQSLLDSDGQTYLHWLLQRPGQSPKRLIYLVSKLGSGVPDRFTGSGSGTDFTLKISRVEAEDLGLYYCWQGTHFPRTFGGGTKLEIK

SEQ ID NO: 153 - protein, 5 aa, Mus musculus
DYSMH

SEQ ID NO: 154 - protein, 7 aa, Mus musculus
GYTFTDY

SEQ ID NO: 155 - protein, 17 aa, Mus musculus
WINTETGEPTYVDDFKG

SEQ ID NO: 156 - protein, 8 aa, Mus musculus
INTETGEP

SEQ ID NO: 157 - protein, 4 aa, Mus musculus
NFDY

SEQ ID NO: 158 - protein, 6 aa, Mus musculus
ATNFDY

SEQ ID NO: 159 - protein, 16 aa, Mus musculus
KSSQSLLDSDGQTYLH

SEQ ID NO: 160 - protein, 11 aa, Mus musculus
QSLLDSDGQTY

SEQ ID NO: 161 - protein, 7 aa, Mus musculus
LVSKLGS

SEQ ID NO: 162 - skipped

SEQ ID NO: 163 - protein, 9 aa, Mus musculus
WQGTHFPRT

SEQ ID NO: 164 - protein, 472 aa, Mus musculus
MGWSWIFLFLLSGTAGVLSEVQLQQSGPELVRPGASVKISCKASGYSFTDYFMHWMKQSHVKSLEWIGRLNPYNGATNYNQNFKDKATLTVDKSSSTAYMELHSLTSEDSAVYYCLSFTTPPEAYWGQGTLVTVSAAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 165 - protein, 239 aa, Mus musculus
MRFSAQLLGLLVLWIPGSTAEIVMTQAAFSNPVTLGTSASISCRSNKSLLYSNGITYLFWYLQRPGQSPQLLIYQMSNLASGVPDRFSGSGSGTDFTLRISRVEAEDVGVYYCAQNLELPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 166 - protein, 117 aa, Mus musculus
EVQLQQSGPELVRPGASVKISCKASGYSFTDYFMHWMKQSHVKSLEWIGRLNPYNGATNYNQNFKDKATLTVDKSSSTAYMELHSLTSEDSAVYYCLSFTTPPEAYWGQGTLVTVSA

SEQ ID NO: 167 - protein, 112 aa, Mus musculus
EIVMTQAAFSNPVTLGTSASISCRSNKSLLYSNGITYLFWYLQRPGQSPQLLIYQMSNLASGVPDRFSGSGSGTDFTLRISRVEAEDVGVYYCAQNLELPWTFGGGTKLEIK

SEQ ID NO: 168 - protein, 5 aa, Mus musculus
DYFMH

SEQ ID NO: 169 - protein, 8 aa, Mus musculus
GYSFTDYF

SEQ ID NO: 170 - protein, 17 aa, Mus musculus
RLNPYNGATNYNQNFKD

SEQ ID NO: 171 - protein, 8 aa, Mus musculus
LNPYNGAT

SEQ ID NO: 172 - protein, 8 aa, Mus musculus
FTTPPEAY

SEQ ID NO: 173 - protein, 10 aa, Mus musculus
LSFTTPPEAY

SEQ ID NO: 174 - protein, 16 aa, Mus musculus
RSNKSLLYSNGITYLF

SEQ ID NO: 175 - protein, 11 aa, Mus musculus
KSLLYSNGITY

SEQ ID NO: 176 - protein, 7 aa, Mus musculus
QMSNLAS

SEQ ID NO: 177 - skipped

SEQ ID NO: 178 - protein, 9 aa, Mus musculus
AQNLELPWT

SEQ ID NO: 179 - protein, 476 aa, Mus musculus
MGWSWIFLFLLSGTAGVLSEVQLQQSGPELVKPGASVRISCKTSGYTFTEYTMHWVKQRLGKSLEWIGGINPNSGNSRFSQKFKDRATLTVDKSSTTAYMELRSLTSEDSAVYFCTRNYGDYGYFAMDYWGQGTSVTVSSAKTTAPSVYPLAPVCGDTTGSSVTLGCLVKGYFPEPVTLTWNSGSLSSGVHTFPAVLQSDLYTLSSSVTVPSSTWPSQTVTCSVAHPASSTTVDKKLEPSGPISTINPCPPCKECHKCPAPNLEGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 180 - protein, 234 aa, Mus musculus
MRTPAQFLGILLLWFPGIKCDIKMTQSPSSMYAFLGERVTIACKASQDIHRYLSWFQQKPGKSPKTLIYRANSLVDGVPSRFSGSGSGQDYSLTISSLEYEDVGIYYCLQFAEFPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 181 - protein, 121 aa, Mus musculus
EVQLQQSGPELVKPGASVRISCKTSGYTFTEYTMHWVKQRLGKSLEWIGGINPNSGNSRFSQKFKDRATLTVDKSSTTAYMELRSLTSEDSAVYFCTRNYGDYGYFAMDYWGQGTSVTVSS

SEQ ID NO: 182 - protein, 107 aa, Mus musculus
DIKMTQSPSSMYAFLGERVTIACKASQDIHRYLSWFQQKPGKSPKTLIYRANSLVDGVPSRFSGSGSGQDYSLTISSLEYEDVGIYYCLQFAEFPLTFGAGTKLELK

SEQ ID NO: 183 - protein, 5 aa, Mus musculus
EYTMH

SEQ ID NO: 184 - protein, 8 aa, Mus musculus
GYTFTEYT

SEQ ID NO: 185 - protein, 17 aa, Mus musculus
GINPNSGNSRFSQKFKD

SEQ ID NO: 186 - protein, 8 aa, Mus musculus
INPNSGNS

SEQ ID NO: 187 - protein, 8 aa, Mus musculus
YGYFAMDY

SEQ ID NO: 188 - protein, 14 aa, Mus musculus
TRNYGDYGYFAMDY

SEQ ID NO: 189 - protein, 17 aa, Mus musculus
KASQDIHRYLSWFQQKP

SEQ ID NO: 190 - protein, 6 aa, Mus musculus
QDIHRY

SEQ ID NO: 191 - protein, 7 aa, Mus musculus
RANSLVD

SEQ ID NO: 192 - skipped

SEQ ID NO: 193 - protein, 9 aa, Mus musculus
LQFAEFPLT

SEQ ID NO: 194 - protein, 461 aa, Mus musculus
MGWSWIFLFLLSGTAGVLSEVQLQQSGPELVKPGASVKISCKASGYSFTDYYMHWVKQSHVKSLEWIGRINPYNGATKYNQNFKDKASLTVDKSSSTAYMELHSLTSEDSAVYYCTRRYDNDDGDYWGQGTTLRVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 195 - protein, 239 aa, Mus musculus
MRFSAQLLGLLVLWIPGSTADIVMTQAAFSNPVTLGTSASISCRSSKSLLYSNGITYLYWYLQKPGQSPQLLIYQMSNLASGVPDRFSSSGSGTDFTLRISRVEAEDVGVYYCAQNLELPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 196 - protein, 118 aa, Mus musculus
EVQLQQSGPELVKPGASVKISCKASGYSFTDYYMHWVKQSHVKSLEWIGRINPYNGATKYNQNFKDKASLTVDKSSSTAYMELHSLTSEDSAVYYCTRRYDNDDGDYWGQGTTLRVSS

SEQ ID NO: 197 - protein, 112 aa, Mus musculus
DIVMTQAAFSNPVTLGTSASISCRSSKSLLYSNGITYLYWYLQKPGQSPQLLIYQMSNLASGVPDRFSSSGSGTDFTLRISRVEAEDVGVYYCAQNLELPWTFGGGTKLEIK

SEQ ID NO: 198 - protein, 5 aa, Mus musculus
DYYMH

SEQ ID NO: 199 - protein, 8 aa, Mus musculus
GYSFTDYY

SEQ ID NO: 200 - protein, 17 aa, Mus musculus
RINPYNGATKYNQNFKD

SEQ ID NO: 201 - protein, 8 aa, Mus musculus
INPYNGAT

SEQ ID NO: 202 - protein, 8 aa, Mus musculus
RYDNDDGD

SEQ ID NO: 203 - protein, 11 aa, Mus musculus
TRRYDNDDGDY

SEQ ID NO: 204 - protein, 16 aa, Mus musculus
RSSKSLLYSNGITYLY

SEQ ID NO: 205 - protein, 11 aa, Mus musculus
KSLLYSNGITY

SEQ ID NO: 206 - protein, 7 aa, Mus musculus
QMSNLAS

SEQ ID NO: 207 - skipped

SEQ ID NO: 208 - protein, 9 aa, Mus musculus
AQNLELPWT

SEQ ID NO: 209 - protein, 460 aa, Mus musculus
MAWVWTLLFLMAAAQRIQAQIQLVQSGPDLKKPGETVKISCKASGYSFTDFSVHWVKQSPGMGLKWMGWINTETGEPTYADDFKGRFAFSLETSASTAYLQINYLKNDDTATYFCARGGRYDDDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 210 - protein, 240 aa, Mus musculus
MESQTQVLISLLFWVSGTCGDIVMTQSPSSLSVSAGEKVTMSCKSSQSLLNSGNQKNYLAWYQQKPGQPPKRLIYGASTRQSGVPDRFTGSGSGTDFTLTISSVQAEDLAVYYCQNDHSYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 211 - protein, 117 aa, Mus musculus
QIQLVQSGPDLKKPGETVKISCKASGYSFTDFSVHWVKQSPGMGLKWMGWINTETGEPTYADDFKGRFAFSLETSASTAYLQINYLKNDDTATYFCARGGRYDDDYWGQGTTLTVSS

SEQ ID NO: 212 - protein, 113 aa, Mus musculus
DIVMTQSPSSLSVSAGEKVTMSCKSSQSLLNSGNQKNYLAWYQQKPGQPPKRLIYGASTRQSGVPDRFTGSGSGTDFTLTISSVQAEDLAVYYCQNDHSYPLTFGAGTKLELK

SEQ ID NO: 213 - protein, 5 aa, Mus musculus
DFSVH

SEQ ID NO: 214 - protein, 8 aa, Mus musculus
GYSFTDFS

SEQ ID NO: 215 - protein, 17 aa, Mus musculus
WINTETGEPTYADDFKG

SEQ ID NO: 216 - protein, 8 aa, Mus musculus
INTETGEP

SEQ ID NO: 217 - protein, 8 aa, Mus musculus
GGRYDDDY

SEQ ID NO: 218 - protein, 10 aa, Mus musculus
ARGGRYDDDY

SEQ ID NO: 219 - protein, 17 aa, Mus musculus
KSSQSLLNSGNQKNYLA

SEQ ID NO: 220 - protein, 12 aa, Mus musculus
QSLLNSGNQKNY

SEQ ID NO: 221 - protein, 7 aa, Mus musculus
GASTRQS

SEQ ID NO: 222 - skipped

SEQ ID NO: 223 - protein, 9 aa, Mus musculus
QNDHSYPLT

SEQ ID NO: 224 - protein, 466 aa, Mus musculus
MAWVWTLLFLMAAAQRIQAQIQLVQSGPDLKNPGETVKISCKASGYSFTDFSVHWVKQSPGIHLKWMGWINTETGEPTYADDFKGRFAFSLETSASTAYLQINYLKNDDTATYFCARGGRYDDDYWGQGTALTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 225 - protein, 240 aa, Mus musculus; identical to SEQ ID NO: 210

SEQ ID NO: 226 - protein, 117 aa, Mus musculus
QIQLVQSGPDLKNPGETVKISCKASGYSFTDFSVHWVKQSPGIHLKWMGWINTETGEPTYADDFKGRFAFSLETSASTAYLQINYLKNDDTATYFCARGGRYDDDYWGQGTALTVSS

SEQ ID NO: 227 - protein, 113 aa, Mus musculus; identical to SEQ ID NO: 212

SEQ ID NO: 228 - protein, 5 aa, Mus musculus
DFSVH

SEQ ID NO: 229 - protein, 8 aa, Mus musculus
GYSFTDFS

SEQ ID NO: 230 - protein, 17 aa, Mus musculus
WINTETGEPTYADDFKG

SEQ ID NO: 231 - protein, 8 aa, Mus musculus
INTETGEP

SEQ ID NO: 232 - protein, 8 aa, Mus musculus
GGRYDDDY

SEQ ID NO: 233 - protein, 10 aa, Mus musculus
ARGGRYDDDY

SEQ ID NO: 234 - protein, 17 aa, Mus musculus
KSSQSLLNSGNQKNYLA

SEQ ID NO: 235 - protein, 12 aa, Mus musculus
QSLLNSGNQKNY

SEQ ID NO: 236 - protein, 7 aa, Mus musculus
GASTRQS

SEQ ID NO: 237 - skipped

SEQ ID NO: 238 - protein, 9 aa, Mus musculus
QNDHSYPLT

SEQ ID NO: 239 - protein, 12 aa, Mus musculus
XGYTFTEYTXXX

SEQ ID NO: 240 - protein, 8 aa, Mus musculus
GYTFTEYT

SEQ ID NO: 241-242 - skipped (2 entries)

SEQ ID NO: 243 - protein, 25 aa, Mus musculus
XXXXXXXXXXXXXXXXXXXXWFPNX

SEQ ID NO: 244 - protein, 11 aa, Mus musculus
XXXXXXXWFPN

SEQ ID NO: 245 - protein, 16 aa, Mus musculus
XXXQSIVHSNGNTYXX

SEQ ID NO: 246 - skipped

SEQ ID NO: 247 - protein, 9 aa, Mus musculus
XQGSHVPWT

SEQ ID NO: 248 - protein, 10 aa, Mus musculus
GYTFTDYXXX

SEQ ID NO: 249 - protein, 17 aa, Mus musculus
XINTETGEPXXXXXXXX

SEQ ID NO: 250 - skipped

SEQ ID NO: 251 - protein, 16 aa, Mus musculus
XXXQSLLDSDGQTYXX

SEQ ID NO: 252-254 - skipped (3 entries)

SEQ ID NO: 255 - protein, 17 aa, Mus musculus
XLNPYNGATXXXXXXXX

SEQ ID NO: 256 - protein, 14 aa, Mus musculus
XXXXXXFTTPPEAY

SEQ ID NO: 257 - protein, 16 aa, Mus musculus
XXXKSLLYSNGXTYXX

SEQ ID NO: 258 - skipped

SEQ ID NO: 259 - protein, 11 aa, Mus musculus
XAQNLELPWTX

SEQ ID NO: 260 - skipped

SEQ ID NO: 261 - protein, 30 aa, Mus musculus
XXNPNSGNSRFSXXXXXXXXXXXXXXXXXX

SEQ ID NO: 262 - protein, 10 aa, Mus musculus
NPNSGNSRFS

SEQ ID NO: 263 - protein, 16 aa, Mus musculus
XXXXNYGDYGYXAMDY

SEQ ID NO: 264 - protein, 8 aa, Mus musculus
YGYXAMDY

SEQ ID NO: 265 - protein, 11 aa, Mus musculus
XXXQDIHRYXX

SEQ ID NO: 266 - protein, 17 aa, Mus musculus
KASQDIHRYLSXXXXXX

SEQ ID NO: 267 - skipped

SEQ ID NO: 268 - protein, 9 aa, Mus musculus
XQFAEFPLT

SEQ ID NO: 269 - skipped

SEQ ID NO: 270 - protein, 5 aa, Mus musculus
YSFTD

SEQ ID NO: 271 - skipped

SEQ ID NO: 272 - protein, 5 aa, Mus musculus
INPYN

SEQ ID NO: 273 - protein, 11 aa, Mus musculus
XXXXXNXDGDX

SEQ ID NO: 274 - protein, 5 aa, Mus musculus
NXDGD

SEQ ID NO: 275 - protein, 16 aa, Mus musculus
XXXKSLLYSNGITYXX

SEQ ID NO: 276 - protein, 11 aa, Mus musculus
KSLLYSNGITY

SEQ ID NO: 277 - skipped

SEQ ID NO: 278 - protein, 11 aa, Mus musculus
XAQNLELPWTX

SEQ ID NO: 279 - skipped

SEQ ID NO: 280 - protein, 17 aa, Mus musculus
XINTETGEPXXXXXXXX

SEQ ID NO: 281 - protein, 10 aa, Mus musculus
XXGGRYDDDY

SEQ ID NO: 282 - protein, 10 aa, Mus musculus
XXXXRYDDDY

SEQ ID NO: 283 - protein, 17 aa, Mus musculus
XXXQSLLNSGNQKNYXX

SEQ ID NO: 284 - protein, 8 aa, Mus musculus
QSLLNSGN

SEQ ID NO: 285 - skipped

SEQ ID NO: 286 - protein, 11 aa, Mus musculus
XXNDHSYPLTX

SEQ ID NO: 287 - skipped

SEQ ID NO: 288 - protein, 17 aa, Mus musculus
XINTETGEPXXXXXXXX

SEQ ID NO: 289 - protein, 10 aa, Mus musculus
XXGGRYDDDY

SEQ ID NO: 290 - protein, 8 aa, Mus musculus
XXXYDDDY

SEQ ID NO: 291 - protein, 17 aa, Mus musculus
XXXQSLLNSGNQKNYXX

SEQ ID NO: 292 - skipped

SEQ ID NO: 293 - protein, 11 aa, Mus musculus
XQNDHSYPLTX